(19) United States
(12) Patent Application Publication
(10) Pub. No.: US 2013/0047985 A1
(43) Pub. Date: Feb. 28, 2013

(54) DRY POWDER INHALER ASSEMBLY AND CONTAINERS

(71) Applicants: David S. Harris, Milton (GB); Matthew D. Allen, London (GB); Amanda J. Fuller, Coton (GB); Nicholas Harrison, Oakington (GB); Karl J. Hewson, Papworth Everard (GB); Peter D. Hodson, Breaston (GB); Scott A. Lewis, Cambridge (GB); Tom A. Oakley, Cambridge (GB); Samantha A. Patterson, Cambridge (GB); Simon J. Smith, Hertford (GB); Rachel V. Striebig, London (GB); Adam J. Stuart, Loughborough (GB)

(72) Inventors: David S. Harris, Milton (GB); Matthew D. Allen, London (GB); Amanda J. Fuller, Coton (GB); Nicholas Harrison, Oakington (GB); Karl J. Hewson, Papworth Everard (GB); Peter D. Hodson, Breaston (GB); Scott A. Lewis, Cambridge (GB); Tom A. Oakley, Cambridge (GB); Samantha A. Patterson, Cambridge (GB); Simon J. Smith, Hertford (GB); Rachel V. Striebig, London (GB); Adam J. Stuart, Loughborough (GB)

(73) Assignee: 3M INNOVATIVE PROPERTIES COMPANY, ST. PAUL, MN (US)

(21) Appl. No.: 13/642,717

(22) PCT Filed: Apr. 21, 2011
(86) PCT No.: PCT/US11/33373

§ 371 Date: Nov. 9, 2012

Related U.S. Application Data
(60) Provisional application No. 61/327,606, filed on Apr. 23, 2010.

(51) Int. Cl. A61M 15/00

(52) U.S. Cl. 128/203.15

(57) ABSTRACT

The present disclosure provides dry powder inhalers, containers for carrying a dose of dry powder medicament particles for dry powder inhalers, and a hinge that, while enjoying broader applicability, may be used in such inhalers.

This disclosure relates generally to inhalation devices for the delivery of dry powder medicament particles. More particularly, the present disclosure relates to containers for use in dry powder inhalers.

A dry powder inhaler (DPI) is a form of drug delivery device used to deliver a medicament in powdered form to the lungs of a user to treat asthma and other respiratory diseases. A user of a dry powder inhaler holds the inhaler to his or her mouth and draws a breath through the device, thereby setting up a flow of air that entrains dry powder medicament particles within the device so that they are drawn into the user's respiratory system. The medicament can be in the form of a free powder or, more commonly, the medicament is bound to carrier particles, such as lactose.

A dry powder inhaler includes a storage compartment containing the medicament, which is often sealed prior to use. That seal is broken by the user and then, via the user's inhalation, the medicament is stirred with the ambient air drawn through the dry powder inhaler so that the medicament particles become airborne. It has been found to be efficient to create a reverse cyclone flow for use in the stirring process of the medicament with the air. The use of a reverse cyclone not only efficiently entrains the medicament within the flow of air, but also separates medicament particles from carrier particles via the air flow patterns created in a reverse cyclone. In effect, the user's inhaled breath causes air to be drawn into a reverse cyclone forming chamber, setting up reverse cyclonic flow. This air flow entrains the dry powder medicament and carrier particles and the high cyclonic shearing forces cause the smaller medicament particles to be stripped from the larger carrier particles. Ideally, the bulk of the smaller medicament particles are carried by the patient's drawn-in breath into the patient's lungs, while the bulk of the larger carrier particles are retained within the reverse cyclone chamber. Examples of dry particle inhalers employing this reverse cyclone particle stirring and separation are disclosed in International Patent Application Publication WO2006/061637 A2 (incorporated by reference in its entirety herein).

### SUMMARY

This disclosure relates to a dry powder inhaler, the relationships and structures of components thereof, including a container for dry power medicament, and a hinge that, while enjoying broader applicability, may be used in such a dry powder inhaler.

In one aspect, a dry power inhaler includes a chamber therein wherein air and entrained dry powder medicament particles can circulate about an axis. The inhaler further includes an outlet tube connectable in fluid communication with the chamber to extend coaxially along the axis and away from the chamber, the tube comprising a first cylindrical section adjacent the chamber wherein air and entrained dry powder medicament particles can move helically about the axis in direction away from the chamber and a second section in fluid communication with the first section wherein air and entrained dry powder medicament particles can move in direction generally parallel to the axis away from the chamber.

In another aspect, a container for carrying a dose of dry powder medicament particles for a dry powder inhaler includes a chamber having a closed bottom and an open top, the chamber having a first lower portion including a flow disruptor, a second intermediate portion adapted for housing reverse cyclone flow therein, and a third upper portion defined in part by an annular shoulder extending radially outwardly within the chamber adjacent to the second portion.

In another aspect, a dry powder inhaler includes a first member comprising a first mating surface, a first air inlet and an air outlet, and a second member comprising a second mating surface adapted for selective engagement with the first mating surface and a chamber formed at least in part for reverse cyclone flow therein. The first and second members are moveable relative to one another between a first position wherein the mating surfaces are spaced apart and a second position wherein the mating surfaces are engaged and portions of the each of the first air inlet and air outlet are within the chamber of the second member.

In another aspect, a dry powder inhaler includes a chamber therein wherein air and entrained dry powder particles can circulate about an axis, and a plurality of air inlet channels in fluid communication with the chamber, wherein each inlet channel is pitched relative to the axis to define a helical flow stream for air entering the chamber from that inlet channel, and wherein the inlet channels are aligned so that their respective helical flow streams are stratified as they axially traverse the chamber.

In another aspect, the disclosure relates to a hinge with a hinge axis about which rotation can occur, wherein an element of the hinge has an ability to flex in direction generally along the axis to allow a first hinge assembly step, and wherein a second hinge assembly step prevents or substantially reduces said ability to flex.

The subject matter of the present disclosure, in its various combinations, either in apparatus or method form, may be characterized by the following list of embodiments:

1. A dry powder inhaler comprising:

a chamber therein wherein air and entrained dry powder medicament particles can circulate about an axis; and

an outlet tube connectable in fluid communication with the chamber to extend coaxially along the axis and away from the chamber, the tube comprising a first cylindrical section adjacent the chamber wherein air and entrained dry powder medicament particles can move helically about the axis in direction away from the chamber and a second section in fluid communication with the first section wherein air and entrained dry powder medicament particles can move in direction generally parallel to the axis away from the chamber.

2. The dry powder inhaler of embodiment 1 wherein the first section of the outlet tube is defined by a single first duct.

3. The dry powder inhaler of embodiment 2 wherein the single first duct has a cylindrical inner wall surface.

4. The dry powder inhaler of embodiment 2 wherein the single first duct comprises a cylindrical outer wall surface.

5. The dry powder inhaler of embodiment 2 wherein the single first duct comprises a frusto-conical outer wall surface.

6. The dry powder inhaler of any of the preceding embodiments wherein the second section of the outlet tube is defined by a plurality of second ducts.

7. The dry powder inhaler of embodiment 6 wherein each of the second ducts in the second section is the same shape, in a plane normal to the axis.

8. The dry power inhaler of embodiment 6 wherein the second section comprises four second ducts.

9. The dry powder inhaler of any of embodiments 2 to 8 wherein the second section of the outlet tube is defined by a plurality of second ducts, and wherein the cross-sectional area, in a plane normal to the axis, of the single first duct of the first section is greater than the combined cross-sectional area, in a plane normal to the axis, of the plurality of second ducts of the second section.

10. The dry powder inhaler of any of embodiments 6 to 9 wherein the length of each of the second ducts is greater than the internal diameter of the first section of the outlet tube.

11. The dry powder inhaler of any of the preceding embodiments wherein the first section of the outlet tube has a first end and comprises a cover piercing element extending outwardly therefrom.

12. The dry powder inhaler of embodiment 11 wherein the cover piercing element extends along the axis.

13. The dry powder inhaler of embodiment 11 or 12 wherein the cover piercing element comprises a central piercing point and a plurality of cover separation wings.

14. The dry power inhaler of embodiment 13 wherein the central piercing point of the cover piercing element extends along the axis and wherein each cover separation wing extends radially outwardly from the cover piercing element.

15. The dry powder inhaler of embodiment 14 wherein an upper portion of each cover separation wing extends axially away from the chamber into the second section of the outlet tube.

16. The dry powder inhaler of embodiment 15 wherein the upper portion of each cover separation wing in the second section of the outlet tube extends further radially outwardly to engage an inner surface of the outlet tube.

17. The dry powder inhaler of embodiment 16 wherein the upper portions of the cover separation wings that extend further radially outwardly define walls separating the second section of the outlet tube into a plurality of ducts.

18. The dry powder inhaler of embodiment 16 or 17 wherein the upper portions of the cover separation wings that extend further radially outwardly do not extend along an entire length of the second section of the outlet tube.

19. The dry powder inhaler of any of embodiments 16 to 18 wherein the upper portions of the cover separation wings that extend further radially outwardly form a cross, in a plane normal to the axis, in the second section of the outlet tube.

20. The dry powder inhaler of embodiment 18 wherein the upper portions of the cover separation wings that extend further radially outwardly are staggered in position along the axis.

21. The dry powder inhaler of any of embodiments 14 to 20 wherein each cover separation wing is at least partially helical in surface form relative to the axis.

22. The dry powder inhaler of any of embodiments 13 to 21 wherein the first end of the outlet tube has a plurality of cover management projections extending therefrom.

23. The dry powder inhaler of embodiment 22 wherein each cover management projection extends parallel to the axis.

24. The dry powder inhaler of embodiment 22 or 23 wherein each cover management projection is aligned relative to adjacent cover separation wings on the cover piercing element.

25. The dry powder inhaler of embodiment 24 wherein the central piercing point of the cover piercing element extends along the axis, wherein each cover separation wing extends radially outwardly from the cover piercing element, and wherein each cover management projection is disposed at a relational angle of between thirty and sixty degrees around the axis relative to each adjacent cover separation wing.

26. The dry powder inhaler of embodiment 25 wherein the relational angle is about forty-five degrees.

27. The dry powder inhaler of any of embodiments 14 to 26 wherein each cover separation wing is in the form of a straight fin.

28. The dry powder inhaler of any of embodiments 14 to 26 wherein the cover separation wings are arranged in the form of a cross, in a plane normal to the axis.

29. The dry powder inhaler of any of the preceding embodiments wherein the first section of the outlet tube has a first end having a wave-shaped edge.

30. The dry powder inhaler of any of the preceding embodiments, further comprising an air inlet channel extending around a portion of the outlet tube and into the chamber.

31. The dry powder inhaler of embodiment 30 wherein the first section of the outlet tube has a first end and the air inlet channel has a first end, and wherein the first end of the first section of the outlet tube extends further into the chamber than the first end of the air inlet channel.

32. The dry powder inhaler of embodiment 30 or 31 wherein the air inlet channel is shaped to cause a single helical flow stream of air to spiral into the chamber from that inlet channel.

33. The dry powder inhaler of any of embodiments 30 to 32 wherein the chamber has an inner annular shoulder and an end of the air inlet channel is formed to selectively mate with the shoulder within the chamber.

34. The dry powder inhaler of embodiment 33 wherein the end of the air inlet channel comprises an outer surface that is generally frusto-conical.

35. The dry powder inhaler of embodiment 33 wherein a spring member urges the air inlet channel and the chamber together.

36. The dry powder inhaler of any of the preceding embodiments, further comprising a plurality of air inlet channels, wherein each inlet channel extends around a respective portion of the outlet tube and into the chamber.

37. The dry powder inhaler of embodiment 36 wherein the plurality of air inlet channels comprises an outer envelope that is generally frusto-conical.

38. The dry powder inhaler of embodiment 36, wherein each inlet channel is shaped to define a helical flow stream for air entering the chamber from that inlet channel.

39. The dry powder inhaler of any of the preceding embodiments wherein the chamber has an internal projection on an end wall thereof opposite to the outlet tube, and wherein the projection is in the general form of a cylindrical peg.

40. The dry powder inhaler of any of the preceding embodiments wherein the chamber comprises a substantially flat end wall that is generally opposed the outlet tube.

41. The dry powder inhaler of embodiment 38 wherein the inlet channels are spaced circumferentially around the outlet tube so that the helical flow streams are stratified as they axially traverse the chamber.

42. The dry powder inhaler of any of embodiments 36 to 41 wherein each inlet channel is shaped to define an air flow stream for air entering the chamber from that inlet channel, and wherein the air flow stream from each inlet channel substantially passes beneath the air flow stream of an adjacent inlet channel as it enters the chamber.

43. The dry powder inhaler of any of embodiments 38 to 42 wherein the air flow stream from each inlet channel spirals into the chamber without substantial impingement on the air flow streams entering the chamber from other inlet channels.

44. The dry powder inhaler of any of the preceding embodiments, further comprising a plurality of air inlet channels, wherein each inlet channel comprises an upstream portion and a downstream portion relative to the direction of air flow through that inlet channel and into the chamber, wherein the upstream portion is in part pitched relative to the axis and has a first linear section and a second arc-shaped section having its inner surface defined by an outer circumferential surface of the outlet tube, and wherein the downstream portion is an annular coaxially extending section having its inner surface defined by the outer circumferential surface of the outlet tube.

45. The dry powder inhaler of embodiment 44 wherein the chamber has an inner annular shoulder and one or more portions of the air inlet channels mate with the inner annular shoulder within the chamber.

46. The dry powder inhaler of embodiment 45 wherein the inner annular shoulder comprises an interior form that is generally frusto-conical.

47. The dry powder inhaler of embodiment 45 wherein the plurality of air inlet channels comprises an outer envelope comprising an exterior form, the exterior form being generally frusto-conical.

48. The dry powder inhaler of any of embodiments 36 to 45 wherein each inlet channel is defined by portions of an upper airways component and a lower airways component.

49. The dry powder inhaler of any of embodiments 36 to 45 wherein each inlet channel is formed within a single airways component.

50. The dry powder inhaler of embodiment 49 wherein each air inlet channel comprises a pitch that varies across its width.

51. The dry powder inhaler of embodiment 50 wherein the pitch is greater at positions farther from the longitudinal axis.

52. The dry powder inhaler of embodiment 48 further comprising a mouthpiece, wherein the upper airways component is adjacent to the mouthpiece and the lower airways component is adjacent to the chamber.

53. The dry powder inhaler of embodiment 52 further comprising one or more bypass channels, wherein the bypass channels direct at least a portion of the inhaled air to the mouthpiece without passing through the chamber.

54. The dry powder inhaler of embodiment 53 wherein the one or more bypass channels direct the at least a portion of the inhaled air into an annular passageway presented around the outlet tube.

55. The dry powder inhaler of embodiment 54 wherein the annular passageway is generally coaxial with the outlet tube.

56. The dry powder inhaler of embodiment 54 wherein the at least a portion of the inhaled air directed into the annular passageway is separated from other inhaled air flowing through the outlet tube.

57. The dry powder inhaler of embodiment 48 or 52 wherein the lower airways component is a molded single integral plastic component having a cover piercing element.

58. The dry powder inhaler of embodiment 48 or 52 wherein the upper airways component, including a cover piercing element, is a unitary molded plastic component.

59. The dry powder inhaler of any of embodiments 48 to 58 wherein each inlet channel is disposed between the upper and lower airways components.

60. The dry powder inhaler of embodiment 59 wherein a portion of each inlet channel has a generally rectangular cross-sectional area, in lateral section relative to the direction of inlet air flow through that inlet channel, with a ceiling, floor and sidewalls.

61. The dry powder inhaler of embodiment 60 wherein the ceiling of each inlet channel portion is defined by the upper airways component and the floor and sidewalls are defined by the lower airways component.

62. The dry powder inhaler of embodiment 60 wherein the ceiling and sidewalls of each inlet channel portion are defined by the upper airways component and the floor of each inlet channel portion is defined by the lower airways component.

63. The dry powder inhaler of any of embodiments 48 to 62 further comprising a mouthpiece, wherein the lower airways component clips onto the upper airways component, and wherein the upper airways component clips onto the mouthpiece.

64. The dry powder inhaler of any of embodiments 48 to 62 further comprising a mouthpiece, wherein the lower airways component clips onto the mouthpiece.

65. The dry powder inhaler of any preceding embodiment further comprising a tapering scroll inlet channel, wherein air can enter the chamber through the tapering scroll inlet channel.

66. The dry powder inhaler of any of embodiments 1 to 64 further comprising a tangential inlet channel with an axis orthogonal to a longitudinal axis of the chamber, wherein air enters the chamber through the tangential inlet channel.

67. A container for carrying a dose of dry powder medicament particles for a dry powder inhaler, the container comprising a chamber having a closed bottom and an open top, the chamber having a first lower portion including a flow disruptor, a second intermediate portion adapted for housing reverse cyclone flow therein, and a third upper portion defined in part by an annular shoulder extending radially outwardly within the chamber adjacent to the second portion.

68. The container of embodiment 67 wherein the second portion is shaped as a conical frustum.

69. The container of embodiment 67 or 68 wherein the third portion is generally cylindrical.

70. The container of embodiment 67 or 68 wherein an interior of the third portion is generally frusto-conical shaped.

71. The container of embodiment 67 or 68 wherein the third portion comprises a square configuration comprising four walls when viewed along a longitudinal axis of the container.

72. The container of embodiment 71 wherein the four walls slope outwardly in a direction towards the open top.

73. The container of embodiment 67 or 68 wherein the third portion comprises an asymmetrical configuration when viewed along a longitudinal axis of the container.

74. The container of any of embodiments 67 to 69 wherein the second portion is deeper than the third portion.

75. The container of any of embodiments 67 to 74 wherein the third portion has a length along the axis of at least 40% of its diameter.

76. The container of any of embodiments 67 to 75 wherein the third portion has a length along the axis of at least 50% of its diameter.

77. The dry powder inhaler of any of embodiments 67 to 76 wherein air enters the chamber through a tapering scroll inlet channel.

78. The dry powder inhaler of any of embodiments 67 to 76 wherein air enters the chamber through an inlet channel comprising an axis orthogonally oriented relative to a longitudinal axis of the chamber.

79. The container of any of embodiments 67 to 78 further comprising a cover extending hermetically over the open top of the chamber.

80. The container of embodiment 79, wherein the cover has a cover alignment tab.

81. The container of embodiment 80, wherein the cover alignment tab projects radially from a central axis of the container.

82. The container of embodiment 79, wherein the cover comprises a pierceable foil layer.

83. The container of embodiment 79, wherein the cover comprises a plastic film layer of low moisture permeability.

84. The container of embodiment 79 or 83, wherein the cover comprises a peelable layer.

85. The container of any of embodiments 67 to 84 further comprising an axially extending rim surface disposed around the open top of the chamber.

86. The container of embodiment 85 further comprising a cover sealed to the rim surface to extend over the open top of the container.

87. The container of any of embodiments 67 to 86 further comprising an alignment confirmation feature disposed on an outer surface of at least one portion of the container.

88. The container of any of embodiments 67 to 87 wherein, on an outer surface thereof, the annular shoulder has an axially extending projection.

89. The container of embodiment 88 wherein the axially extending projection is annular.

90. The container of embodiment 88 or 89 wherein the axially extending projection is deformable.

91. The container of any of embodiments 67 to 90 wherein, on an outer surface thereof, the annular shoulder has a plurality of axially extending projections in the form of radial walls.

92. The container of any of embodiments 67 to 91 further comprising a lid for selectively closing off the open top of the chamber.

93. The container of embodiment 92 wherein the lid is affixed to the chamber and moveable between a first open position relative to the open top of the chamber and a second closed position relative to the open top of the chamber.

94. The container of embodiment 93 wherein the lid is affixed to the chamber via a hinge.

95. The container of embodiment 94 wherein the chamber and lid are a single integral injection molded plastic component, and wherein the hinge is a living hinge.

96. The container of embodiment 79 further comprising a lid for selectively covering the cover without breaching the cover.

97. The container of embodiment 96, wherein the lid moves substantially in a plane parallel to the plane of the cover in order to selectively cover the cover.

98. The container of embodiment 97, wherein the lid slides.

99. The container of embodiment 97, wherein the lid rotates.

100. A dry powder inhaler comprising:

a first member comprising a first mating surface, a first air inlet and an air outlet; and

a second member comprising a second mating surface adapted for selective engagement with the first mating surface and a chamber formed at least in part for reverse cyclone flow therein,

wherein the first and second members are moveable relative to one another between a first position wherein the mating surfaces are spaced apart and a second position wherein the mating surfaces are engaged and portions of the each of the first air inlet and air outlet are within the chamber of the second member.

101. The dry powder inhaler of embodiment 100 wherein the first member further comprises a cover piercing element, and wherein at least a portion of the cover piercing element is disposed within the chamber of the second member when the first and second members are in the second position.

102. The dry powder inhaler of embodiment 100 or 101 wherein the first member and the second member pivot relative to one another between the first and second positions.

103. The dry powder inhaler of embodiment 100 or 101 wherein the reverse cyclone flow occurs about an axis, and wherein the first member and the second member translate axially relative to one another between the first and second positions.

104. The dry powder inhaler of embodiment 101 wherein the reverse cyclone flow occurs about an axis that extends along a center line of the chamber, wherein the first member and/or the second member comprise alignment features that ensure axial movement of the cover piercing element along the axis as the members move between their first and second positions.

105. The dry powder inhaler of embodiment 101 or 104 wherein the chamber has a cover thereon, and wherein the first member and the second member move relative to one another such that a leading portion of the cover piercing element first contacts the cover at the center of the cover.

106. The dry powder inhaler of embodiment 101 or 104 wherein the reverse cyclone flow occurs about an axis that extends along a center line of the chamber, and wherein the first member and the second member move relative to one another such that a leading portion of the cover piercing element lies along the axis when the first and second members are in the second position.

107. The dry powder inhaler of embodiment 105 wherein the leading portion of the cover piercing element first contacts the cover when the first member is in a third position relative to the second member, the third position being between the first and second positions, wherein the first member and the second member pivot relative to one another between the first, third and second positions, respectively, and wherein the pivoting occurs around a hinge axis normal to an axis of the chamber around which the reverse cyclone flow occurs, the hinge axis being positioned at a height that is approximately midway between the height of the leading portion of the cover piercing element when the first member is in the third position and the height of the leading portion of the cover piercing element when the first member is in the second position, said heights all being measured parallel to the chamber axis.

108. The dry powder inhaler of any of embodiments 100 to 107 wherein the second member has a body portion, and wherein the chamber of the second member comprises a container separable from the body portion.

109. The dry powder inhaler of embodiment 108 wherein the body portion comprises a receptacle shaped to receive and retain the container.

110. The dry powder inhaler of embodiment 109 wherein the receptacle and the container are so shaped and arranged such that containers of more than one internal size can be suitably received and retained in the receptacle.

111. The dry powder inhaler of any of embodiments 108 to 110 wherein the container comprises a cup segment and a cover sealed thereover.

112. The dry powder inhaler of any of embodiments 100 to 111, wherein the first member comprises a second air inlet, and wherein a portion of the second air inlet is within the chamber of the second member when the first and second members are in the second position.

113. The dry powder inhaler of embodiment 112 wherein each air inlet is formed to define a helical flow stream for air entering the chamber from that air inlet.

114. The dry powder inhaler of embodiment 113 wherein the air inlets are formed to define stratified helical flow streams for air within the chamber.

115. The dry powder inhaler of any of embodiments 100 to 114 wherein the first and second members are configured to enable the first and second members to be clipped together in the second position.

116. The dry powder inhaler of embodiment 115 wherein the second mating surface is operably urged to be adjacent to the first mating surface by a spring member when the first and second members are in their second position.

117. The dry powder inhaler of embodiment 116 wherein the spring member is a metal coil spring.

118. The dry powder inhaler of any of the preceding embodiments, further comprising a plurality of air inlet channels, wherein each inlet channel comprises an upstream portion and a downstream portion relative to the direction of air flow through that inlet channel and into the chamber, wherein the upstream portion is in part pitched relative to the axis and has a first linear section and a second arc-shaped section having its inner surface defined by an outer circumferential surface of the outlet tube, and wherein the downstream portion is an annular coaxially extending section having its inner surface defined by the outer circumferential surface of the outlet tube.

119. The dry powder inhaler of embodiment 19 wherein the chamber has an inner annular shoulder and one or more portions of the air inlet channels mate with the inner annular shoulder within the chamber.

120. The dry powder inhaler of embodiment 119 wherein the spring member is a compression spring.

121. The dry powder inhaler of embodiment 119 wherein the spring member is a metal coil spring.

122. A dry powder inhaler comprising:

a chamber therein wherein air and entrained dry powder particles can circulate about an axis; and

a plurality of air inlet channels in fluid communication with the chamber, wherein each inlet channel is pitched relative to the axis to define a helical flow stream for air entering the chamber from that inlet channel, and wherein the inlet channels are aligned so that their respective helical flow streams are stratified as they axially traverse the chamber.

123. The dry powder inhaler of embodiment 122 wherein the flow stream from each air inlet channel substantially passes beneath the flow stream of an adjacent air inlet channel as it enters the chamber.

124. The dry powder inhaler of embodiment 122 or 123 wherein the flow stream from each air inlet channel spirals into the chamber without substantial impingement on the flow streams entering the chamber from other air inlet channels.

125. The dry powder inhaler of any of embodiments 122 to 124 wherein the chamber has an inner annular shoulder and one or more portions of the air inlet channels mate with the inner annular shoulder within the chamber.

126. The dry powder inhaler of embodiment 125 wherein the one or more portions of the air inlet channels comprise a frusto-conical outer wall surface.

127. The dry powder inhaler of embodiment 125 wherein the one or more portions of the air inlet channels comprise a frusto-conical inner wall surface.

128. The dry powder inhaler of any of embodiments 122 to 127 wherein each inlet channel is defined by portions of an upper airways component and a lower airways component.

129. The dry powder inhaler of any of embodiments 122 to 128 further comprising a mouthpiece, wherein the upper airways component is adjacent to the mouthpiece and the lower airways component is adjacent to the chamber.

130. The dry powder inhaler of embodiment 129 wherein the lower airways component is a molded single integral plastic component having a cover piercing element.

131. The dry powder inhaler of any of embodiments 122 to 130 wherein the air inlet channels are generally helically shaped for substantially their entire length.

132. The dry powder inhaler of any of embodiments 122 to 131 wherein the air inlet channels are a unitary molded plastic component.

133. The dry powder inhaler of embodiment 131 wherein each air inlet channel comprises a pitch that varies across its width.

134. The dry powder inhaler of embodiment 133 wherein the pitch is greater at positions farther from the longitudinal axis.

135. The dry powder inhaler of embodiment 128 wherein each inlet channel is disposed between the upper and lower airways components.

136. The dry powder inhaler of embodiment 135 wherein a portion of each inlet channel has a generally rectangular cross-sectional area, in lateral section relative to the direction of inlet air flow through that inlet channel, with a ceiling, floor and sidewalls.

137. The dry powder inhaler of embodiment 136 wherein the ceiling of each inlet channel portion is defined by the upper airways component and the floor and sidewalls are defined by the lower airways component.

138. The dry powder inhaler of embodiment 136 wherein the ceiling and sidewalls of each inlet channel portion are defined by the upper airways component and the floor of each inlet channel portion is defined by the lower airways component.

139. The dry powder inhaler of embodiment 128 further comprising a mouthpiece, wherein the lower airways component clips onto the upper airways component, and wherein the upper airways component clips onto the mouthpiece.

140. The dry powder inhaler of embodiment 128 further comprising a mouthpiece, wherein the lower airways component clips onto the mouthpiece.

141. The dry powder inhaler of embodiment 128 wherein each inlet channel comprises an upstream portion and a downstream portion relative to the direction of air flow through that inlet channel and into the chamber, wherein the upstream portion is in part pitched relative to the axis and has a first linear section and a second arc-shaped section having its inner surface defined by an outer circumferential surface of the outlet tube, and wherein the downstream portion is an annular coaxially extending section having its inner surface defined by the outer circumferential surface of the outlet tube.

142. The dry powder inhaler of any of embodiments 122 to 141 wherein the chamber comprises an internal cylindrical projection mounted coaxially on an end wall generally opposed the outlet tube.

143. The dry powder inhaler of any of embodiments 122 to 141 wherein the chamber comprises a substantially flat end wall generally opposed the outlet tube.

144. A hinge with a hinge axis about which rotation can occur, wherein an element of the hinge has an ability to flex in direction generally along the axis to allow a first hinge assembly step, and wherein a second hinge assembly step prevents or substantially reduces said ability to flex.

145. The dry powder inhaler of any of embodiments 1 to 29 in which a separate inhaled airflow bypasses the chamber.

146. The dry powder inhaler of any of embodiments 1 to 29 wherein the separate inhaled airflow which bypasses the chamber meets the air and entrained dry powder medication particles from the chamber part way along the outlet tube.

147. The dry powder inhaler of any of embodiments 1 to 29 or 145 wherein the separate inhaled airflow which bypasses the chamber and the air and entrained dry powder medication particles from the chamber remain separate until downstream of the end of the outlet tube.

This summary is provided to introduce a selection of concepts in a simplified form that are further described below in the Detailed Description. This summary is not intended to identify key features or essential features of the claimed subject matter, is not intended to describe each disclosed embodiment or every implementation of the claimed subject matter, and is not intended to be used as an aid in determining the scope of the claimed subject matter. Many other novel advantages, features, and relationships will become apparent as this description proceeds. The figures and the description that follow more particularly exemplify illustrative embodiments.

### BRIEF DESCRIPTION OF THE DRAWINGS

The disclosed subject matter will be further explained with reference to the attached figures, wherein like structure is referred to by like reference numerals throughout the several views.

FIG. 1 is a side schematic view of air flow in and out of a reverse cyclone chamber for a dry powder inhaler of the present disclosure.

FIG. 2 is a side schematic view of air flow in, out, and within the reverse cyclone chamber of FIG. 1.

FIG. 3A is a top schematic view of air flow in and out of the reverse cyclone chamber of FIG. 2, as taken along lines 3a-3a in FIG. 2.

FIG. 3B is a sectional schematic view of air flow in and out of the reverse cyclone chamber, as taken along lines 3b-3b in FIG. 2.

FIG. 4 is a top isometric illustration of an exemplary dry powder medicament container of the present disclosure, with a lid in an open position.

FIG. 5 is a top isometric illustration of the exemplary dry powder medicament container of FIG. 4, with its cover breached after use, but with its lid still in the open position.

FIG. 6 is a top isometric illustration of the exemplary dry powder medicament container of FIG. 4, with its lid in a closed position over the breached cover.

FIG. 7 is a bottom isometric illustration of the exemplary dry powder medicament container of FIG. 4, with its lid in the closed position.

FIG. 8 is a top isometric illustration of an exemplary dry powder inhaler assembly of the present disclosure.

FIG. 9 is a top isometric illustration of the dry powder inhaler assembly of FIG. 8, depicting a base member holding a dry powder medicament container, with a medicament container piercing and mouthpiece assembly movably attached thereto and pivoted to an open, container insertion/removal position, and with a cap member for the base member movably attached thereto and pivoted to an open position.

FIG. 10 is a top isometric illustration of the dry powder assembly of FIG. 8, with the container piercing and mouthpiece assembly pivoted to a closed position onto the base member, and with the cap member in its open position.

FIG. 11 is a top exploded isometric illustration of the dry powder inhaler assembly of FIG. 8.

FIG. 12 is a side exploded sectional illustration of the dry powder inhaler assembly of FIG. 8.

FIG. 13 is a top sectional isometric illustration of the dry powder assembly of FIG. 8, with its base member, medicament container piercing and mouthpiece assembly and cap member arranged as depicted in FIG. 9.

FIGS. 14-25 illustrate, in sectional views, the sequence of use of the illustrative dry powder inhaler assembly of FIG. 8, where the dry powder inhaler assembly is closed (FIG. 14), being opened (FIGS. 15-16), loaded with a container of dry powder medicament (FIG. 17), used to access the medicament in the container (FIGS. 18-21), again opened (FIG. 22) for removal of the used container (FIG. 23), and closed for storage and/or transport (FIGS. 24-25).

FIG. 26 is an enlarged sectional view of the lower airways component of FIGS. 8-25, in conjunction with an associated dry powder medicament container, illustrating some of the air flow streams therethrough, and with some parts removed and broken away for clarity of illustration.

FIG. 27 is a top enlarged sectional isometric view of the components of FIG. 26.

FIG. 28 is an enlarged, partial, top sectional isometric illustration of a receptacle in the base member of the dry powder inhaler assembly of FIG. 8, depicting how that receptacle is adapted for reception of a dry powder medicament container therein.

FIG. 29 is an enlarged, partial, side sectional view of a receptacle such as that depicted in FIG. 28, depicting a container now disposed within the receptacle.

FIG. 30 is a top isometric illustration of an alternative embodiment of a dry powder inhaler assembly of the present disclosure.

FIG. 31 is a top exploded isometric illustration of the dry powder inhaler assembly of FIG. 30.

FIGS. 32 and 33 illustrate, in sectional views, the dry powder inhaler assembly of FIGS. 30 and 31, in an open position prior to use (FIG. 32) and in a closed position ready for use (FIG. 33).

FIG. 34A is a top exploded isometric illustration of an exemplary container piercing and reverse cyclone air inlet and outlet assembly of the present disclosure.

FIG. 34B is a top assembly isometric illustration of the components of FIG. 34A.

FIG. 35A is a bottom exploded isometric illustration of the components of FIG. 34A.

FIG. 35B is a bottom assembly isometric illustration of the components of FIG. 35A.

FIG. 36A is a side exploded isometric illustration of the components of FIG. 34A.

FIG. 36B is a side assembly isometric illustration of the components of FIG. 36A.

FIG. 36C is a side view of the assembly of FIG. 36B, rotated 90° to the left about a central axis thereof.

FIG. 37 is a bottom plan view of a lower airways component as illustrated in FIGS. 34A-36C.

FIG. 38 is a top plan view of the lower airways component of FIG. 37.

FIG. 38A is a sectional view as taken along lines A-A in FIG. 38.

FIG. 38B is a sectional view as taken along lines B-B in FIG. 38.

FIG. 38C is a sectional view as taken along lines C-C in FIG. 38.

FIG. 39 is a top isometric illustration of an alternative embodiment of a dry powder medicament container of the present disclosure.

FIG. 40 is a bottom isometric illustration of the dry powder medicament container of FIG. 39.

FIGS. 41A-41B illustrate the piercing of the dry powder medicament container of FIGS. 39 and 40 in an exemplary dry powder inhaler assembly of the present disclosure.

FIG. 41C is a side exploded sectional illustration similar to that of FIG. 12, but including the alternative dry powder medicament container of FIGS. 39 and 40.

FIG. 42 schematically illustrates elements of the relationship of the motion of a leading portion of a cover piercing element relative to a central axis A of the dry powder inhaler assembly and a hinge axis B of the dry powder inhaler assembly.

FIG. 43 is an enlarged, partial top isometric illustration of a portion of a body portion of the dry powder inhaler assembly of FIG. 9.

FIG. 44 is a top exploded isometric illustration of two components of the dry powder inhaler assembly of FIG. 9.

FIG. 45 is a top isometric illustration of a subassembly of the dry powder inhaler assembly of FIG. 9.

FIG. 46 is an enlarged, partial top isometric illustration of a lower member of the dry powder inhaler assembly of FIG. 9.

FIG. 47 is a top isometric illustration of a second subassembly of the dry powder inhaler assembly of FIG. 9.

FIG. 48 is a schematic sectional view of an exemplary air inlet channel.

FIG. 49 is a schematic sectional view of an alternative configuration for an air inlet channel.

FIG. 50 is a top isometric illustration of an alternative dry powder medicament container of the present disclosure.

FIGS. 51A and 51B are a top isometric illustration and a top plan view, respectively, of another dry powder medicament container of the present disclosure.

FIG. 52 is a top isometric illustration of another dry powder medicament container of the present disclosure.

FIG. 53 is a top sectional isometric illustration of a further embodiment of a dry powder inhaler of the present disclosure.

FIG. 54 is a sectional view of the dry powder inhaler of FIG. 53.

FIG. 55 is a top isometric view of an inner airways component useable in the dry powder inhaler of FIGS. 53-54.

FIG. 56 is a top isometric view of the inner airways component of FIG. 55 with a lower outer sheath thereof cut away (not depicted) to depict helical airway dividers.

FIG. 57 is a top exploded isometric illustration of a further embodiment of a dry powder inhaler of the present disclosure.

FIG. 58 is a top sectional isometric assembled view of the dry powder inhaler of FIG. 57.

FIG. 59 is a sectional view of the dry powder inhaler of FIGS. 57-58.

FIG. 60 is a sectional view of a modified embodiment of the dry powder inhaler of FIGS. 57-59.

FIG. 61 is a sectional view of a lower member, a lower airways component, and an upper airways component of a further embodiment of a dry powder inhaler assembly of the present disclosure.

FIG. 62 is a top sectional isometric view of the lower member, lower airways component, and upper airways component of the dry powder inhaler of FIG. 61.

FIG. 63 is a top sectional isometric view of the lower member and lower airways component of the dry powder inhaler of FIGS. 61-62.

FIG. 64 is a top isometric view of the lower airways component of FIGS. 61-63.

FIG. 65 is a top plan view of the lower airways component of FIGS. 61-64.

FIG. 66 is a top sectional isometric view of the lower member and lower airways component of a further embodiment of the dry powder inhaler of the present disclosure.

FIG. 67 is a top isometric view of the lower airways component of FIG. 66.

FIG. 68 is a top plan view of the lower airways component of FIGS. 66-67.

While the above-identified figures set forth several embodiments of the disclosed subject matter, other embodiments are also contemplated, such as those noted in the disclosure. In all cases, this disclosure presents the disclosed subject matter by way of representation and not by limitation. The figures are schematic representations, for which reason the configuration of the different structures, as well as their relative dimensions, serves illustrative purposes only. Numerous other modifications and embodiments can be devised by those skilled in the art, which other modifications and embodiments fall within the scope and spirit of the principles of this disclosure.

### DETAILED DESCRIPTION

The terms “inhaler” or “inhaler assembly”, as used herein, refer to any device that is suitable for the administration, to the lungs, of a medicinal formulation in the form of a dry powder.

The terms “upstream” and “downstream”, as used herein, refer to the direction of flow of the air, in use, through the inhaler.

When in the following terms such as “upper” and “lower”, “top” and “bottom”, “right” and “left”, “horizontal” and “vertical”, or similar relative expressions are used, these terms only refer to the appended figures and not to an actual situation of use.

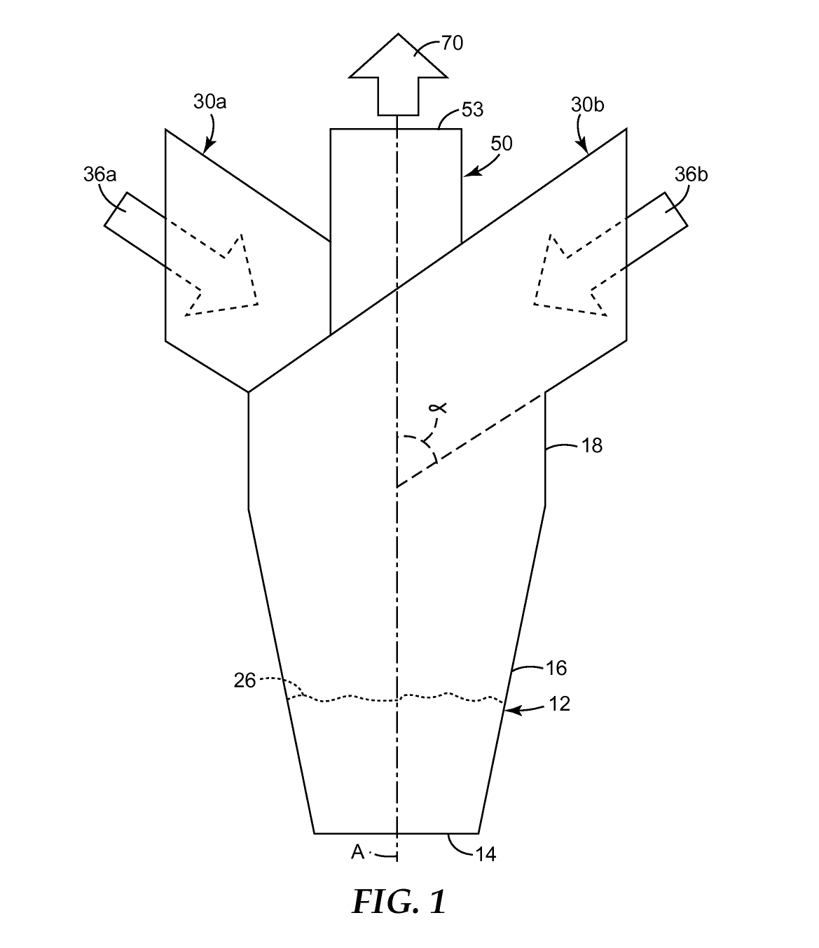

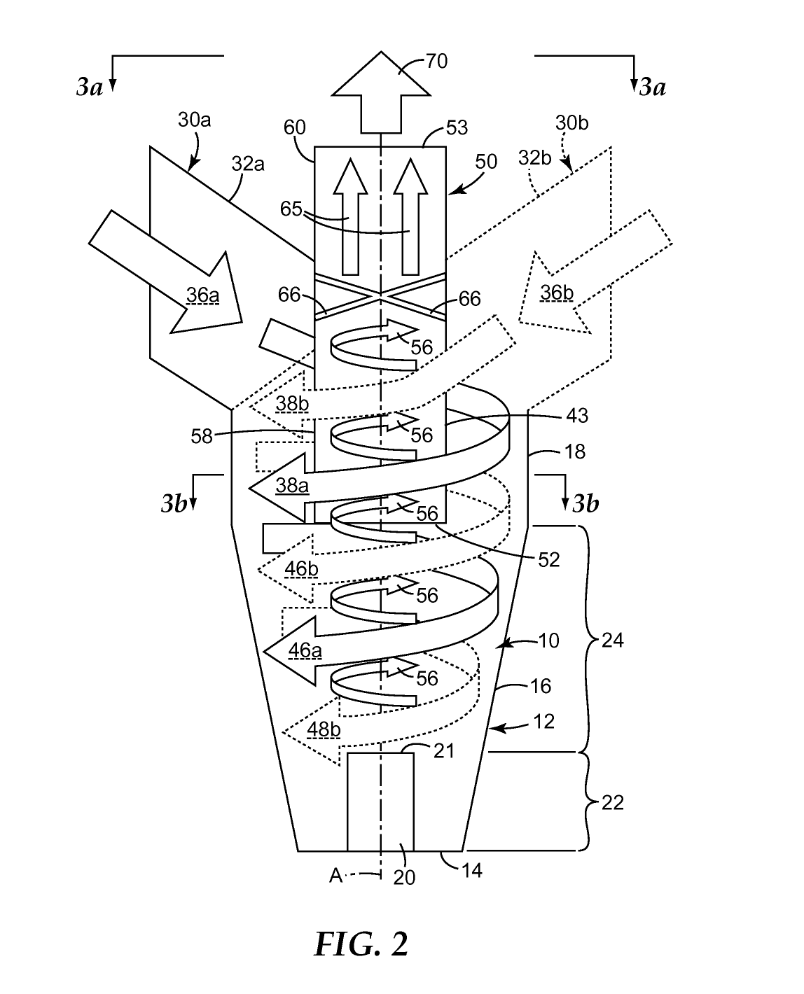

FIGS. 1-3B illustrate, schematically, the structure of a reverse cyclone chamber 10 (FIGS. 1-2) and its associated inlets and outlets for use in a dry powder inhaler assembly. The chamber 10 is defined within an enclosure 12 having a bottom wall 14, a conical frustum shaped upright wall portion 16 (FIGS. 1-2), and a generally cylindrical wall portion 18 atop the wall portion 16. As illustrated in FIG. 2, in embodiments, a flow disruptor 20 in the form of an internal projection (such as a cylindrical peg having a flow disruptor top surface 21) is disposed on the bottom wall 14 and extends upwardly into the chamber 10, thereby defining a first lower segment 22 of the wall portion 16 (having a conical frustum shape and surrounding the flow disruptor 20) and a second upper segment 24 of the wall portion 16 (also having a conical frustum shape). The chamber 10, its respective enclosure 12, and the flow disruptor 20 are disposed on a common axis A (i.e., are coaxially arranged).

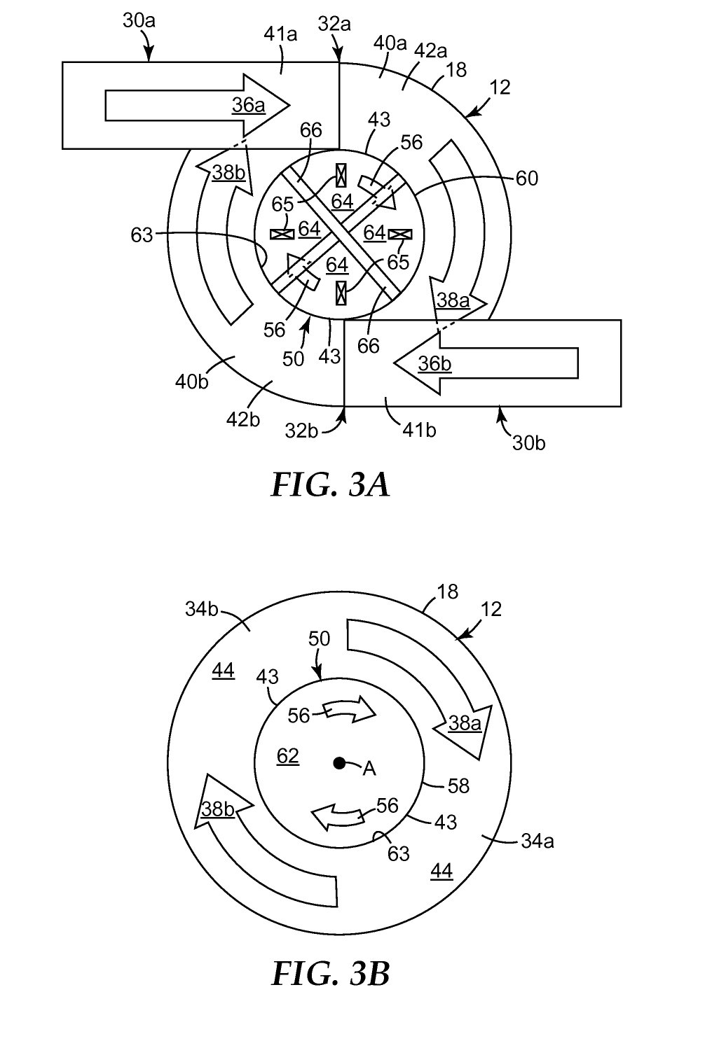

A quantity of dry powder medicament particles is, prior to use, resident within the chamber 10, such as illustrated by the particulate fill line 26 in FIG. 1. The bottom of the chamber 10 is closed, via bottom wall 14, while the top of the chamber 10 is open, and in fluid communication with one or more inlet channels 30a and 30b. Each inlet channel 30a, 30b, has an upstream portion 32a, 32b, and a downstream portion 34a, 34b (FIG. 3B), relative to the direction of air flow through that inlet channel and into the chamber 10, as represented by air flow arrows 36a, 36b and 38a, 38b, respectively. The upstream portion 32a, 32b of each air inlet channel 30a, 30b is in part pitched relative to the axis A (as illustrated by FIGS. 1 and 2) at an acute angle, such as angle α. Each upstream portion 32a, 32b leads to an arc-shaped opening 40a, 40b that defines a fluid communication port between the upstream portion 32a, 32b and the downstream portion 34a, 34b of each air inlet channel 30a, 30b.

An outlet tube 50 is coaxially disposed on the axis A and has a first end 52 that extends into the chamber 10, with the outlet tube 50 extending to a second end 53 that is above and extends away from the chamber 10. The outlet tube 50 is disposed between the air inlet channels 30a and 30b.

As seen in FIG. 3A, the upstream portion 32a, 32b of each air inlet channel 30a, 30b has a first linear section 41a, 41b and a second arc-shaped section 42a, 42b, with the latter having its inner surface defined by an outer circumferential surface 43 of the outlet tube 50. The downstream portion 34a, 34b of each air inlet channel 30a, 30b is an annular coaxially extending section 44 having its inner surface defined by the outer circumferential surface 43 of the outlet tube 50 (see FIG. 3B). As depicted, the downstream portions 34a, 34b of each air inlet channel 30a, 30b share the common annular section 44, although as explained below, the air flow streams from the air inlet channels are substantially free from interference with one another as they traverse the annular section 44 and subsequently enter the frustoconical part of the chamber 10. The annular section 44 extends into the wall portion 18 of the enclosure 12, and extends around a portion of the outlet tube 50. Thus, each air inlet channel 30a, 30b extends around a respective portion of the outlet tube 50 and into the chamber 10. Each air inlet channel 30a, 30b, is shaped to define a helical flow stream for air entering the chamber 10 from its respective inlet channel. The inlet channels 30a, 30b are spaced circumferentially around the outlet tube 50 so that the helical flow streams are stratified as they traverse the chamber 10 downwardly and about the axis A. This stratification of helical flow streams of inlet air is illustrated in FIG. 2, with the air flow stream from the air inlet channel 30a illustrated by the flow arrow 38a (in solid lines) and the air flow stream from inlet channel 30b illustrated by the air flow arrow 38b (in dotted lines). As depicted, the air flow stream from each inlet channel substantially passes beneath the air flow stream of the adjacent inlet channel as it enters the chamber 10. The air flow stream from each inlet channel spirals into the chamber 10 without substantial impingement on the air flow streams entering the chamber 10 from other inlet channels. Each air inlet channel is shaped to cause a single helical flow stream of air to spiral into the chamber 10 from that inlet channel.

As the helical air flow streams from the inlet channels spiral downwardly along the inner walls of the chamber 10, and in particular conical frustum shaped portion 16, a reverse cyclone flow pattern in the chamber 10 is established. The reverse cyclone flow pattern referred to herein has a particular meaning distinct from the general usage of the term cyclone in the art to mean any form of circulating air. A reverse cyclone flow is one in which the air circulates in two generally coaxial columns in opposite axial directions. These two columns include an outward downwardly spiraling “free” vortex and an inner, upwardly spiraling “forced” vortex. These counteracting vortexes create a substantial fluctuation in tangential velocity across the width of the chamber 10. The tangentially entering air and the cylindrical upper wall portion 18 set up a bulk circulation of air around the periphery of the chamber 10. As the incoming air from the air inlet channels 30a, 30b is pitched downwardly, the air flow streams take on a shallow downward spiral, as illustrated by air flow arrows 38a, 38b, 46a, 46b and 48b (FIG. 2). Due to conservation of angular momentum, the rotational velocity of this free vortex increases as the air flow is constricted by the tapering inner surface of the conical frustum shaped portion 16 of the chamber 10. As the free vortex reaches the top surface 21 of the flow disruptor 20, it is effectively reflected to form a tight forced vortex inside the free vortex and travels back up the axis A of the chamber 10 while rotating in the same direction (as represented by air flow arrows 56).

The first end 52 of the outlet tube 50 forms a vortex finder, which effectively defines a maximum cut-off circulation radius for entrained particles to exit the chamber 10. Particles circulating at a radius greater than that of the vortex finder (outlet tube 50) will not escape but will either fall back into the cyclone or fall to the base of the chamber 10.

The outlet tube 50 has a first cylindrical lower section 58 and a second upper section 60. The first cylindrical lower section 58 is adjacent the chamber 10, and air and entrained dry powder medicament particles can move helically about the axis A in direction away from the chamber 10 within the first lower section 58, as illustrated by the arrows 56 therein.

The first lower section 58 is defined by a single air outlet duct 62, as seen in FIG. 3B. The single outlet duct 62 has a cylindrical inner wall surface 63, with a circular cross-section in a plane normal relative to the axis A.

The second upper section 60 of the outlet tube 50 is in fluid communication with the first section 58 and is defined by one or more second ducts 64. In a selected embodiment, there is a plurality of second ducts 64. As illustrated in FIG. 3A, in embodiments, each of the second ducts 64 in the second section 60 of the outlet tube 50 is the same shape, in a plane normal to the axis A. In the embodiment illustrated in FIG. 3A, the second section 60 includes four second ducts 64. In embodiments, the area, in a plane normal to the axis A, of the single first duct 62 of the first section 58 of the outlet tube 50 is greater than the combined area, in a plane normal to the axis A, of the plurality of second ducts 64 of the second section 60 of the outlet tube 50. In embodiments, the length of each of the second ducts 64 is greater than the internal diameter of the first section 58 of the outlet tube 50. As already mentioned, the outlet tube 50 has its second section 60 in fluid communication with its first section 58. In the second section 60, air and entrained dry powder medicament particles can move in direction generally parallel to the axis A away from the chamber 10, such as illustrated by air flow arrows 65 in FIGS. 2 and 3A.

In the second section 60 of the outlet tube 50, the second ducts 64 are defined by the inner circumferential wall 63 of the outlet tube 50 and one or more divider walls 66 extending across the interior of the second section 60. In embodiments, the divider walls 66 are disposed in the form of a cross, such as seen in FIG. 3A. The air flow exiting the outlet tube 50 at its second end 53 is represented by air flow arrow 70, and is arranged (e.g., via a suitable mouthpiece) to directly enter the respiratory system of a user via inhalation.

Because of the effectiveness of the reverse cyclone stirring and separation activity taking place within the chamber 10, the number of dry powder medicament particles entrained within the air flow 70 entering a user's respiratory system is increased. The effectiveness of a reverse cyclone is further increased by use of the flow disruptor 20. The effectiveness of the reverse cyclone is also further enhanced by the effectiveness of the unique air flow inlet arrangement illustrated in FIGS. 1-3B, allowing the stratification of downwardly directed inflow air streams from two or more air flow inlets. Effectiveness of dry powder particulate distribution is further enhanced by the two stage outlet tube 50, wherein the upwardly spiraling air flow stream 56 is converted to an air flow stream 65 moving generally parallel to the axis A.

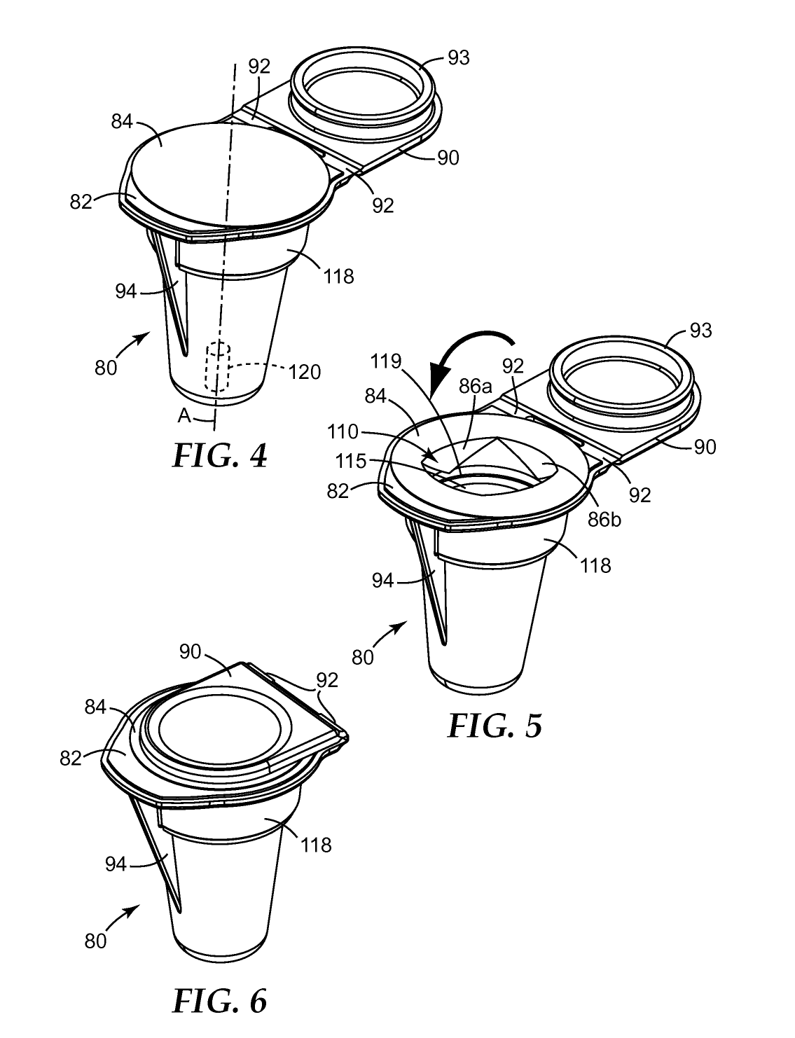

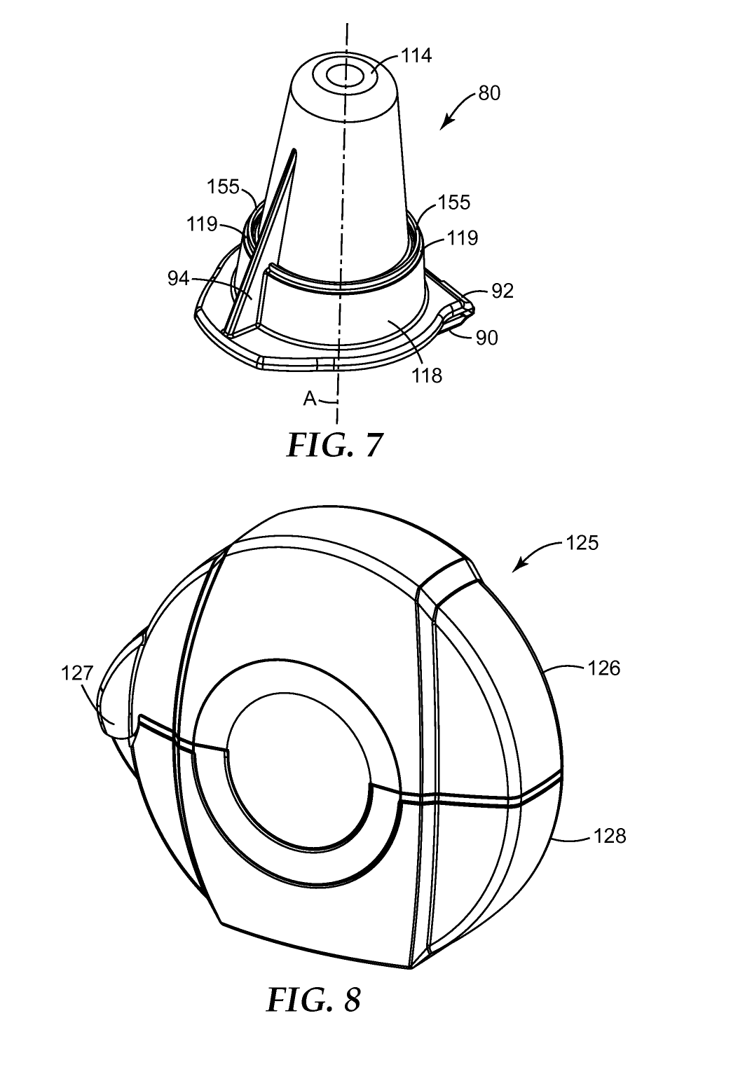

In embodiments of the dry powder inhaler assembly of the present disclosure, chamber 10 is defined within a single use and disposable dry powder container 80, as illustrated in FIGS. 4-7. The container 80 has a chamber 110 therein (see FIG. 5) having a closed bottom 114 (see FIG. 7) and an open top 115 (see FIG. 5). In the same general manner as illustrated in FIGS. 1-4, the chamber 110 of the container 80 has a first lower portion which includes a flow disruptor 120 (see FIG. 4), a second intermediate portion adapted for housing reverse cyclone flow therein (conical frustum shaped), and a third upper portion 118 that is generally cylindrical. In the container 80, the third portion 118 is defined in part by an annular shoulder 119 extending radially outwardly within the chamber adjacent a top of the second portion (see FIG. 5). In embodiments, the second portion is deeper (as disposed along the axis A) than the third portion 118.

In embodiments, the container has a radially extending rim surface 82 disposed around the open top 115 of the chamber 110. A cover 84 is sealed to the rim surface 82 to extend over the open top 115 of the container 80. The cover thus extends hermetically over the open top 115 of the chamber 110. In embodiments, the cover 84 is a foil layer. In other embodiments, the cover 84 is a plastic film layer of low moisture permeability. The cover, in whatever form it takes, is intended to provide a physical barrier over its respective chamber and a moisture barrier to maintain the medicament particles both within the chamber as well as dry. However, the cover 84 is adapted to be breached (e.g., pierced or otherwise removed) in a controlled manner to allow access to the dry powder medicament within the container 80. FIG. 5 illustrates the cover 84 as breached during use of a dry powder inhaler, wherein one or more cover flaps 86a, 86b are separated and pressed against an inner wall of the third portion 118 of the chamber 110 (additional cover flaps not seen in FIG. 5; but see cover flap 86d in FIG. 21). The depth of the third portion 118 is long enough axially to accommodate the length of what may reasonably be expected for a cover flap hanging down from the rim 82 of the container 80, so that no portion of that cover flap extends into the chamber 110 during use (see, e.g., the relationship of the cover flaps 86d and 86b to the container 80 in FIG. 21). In embodiments, the depth of the third portion, as taken in an axial direction, is at least 40% of a diameter of the third portion at a top extent thereof. In another embodiment, the depth of the third portion is at least 50% of that diameter.

In embodiments, the container 80 has a lid 90 for selectively closing off the open top 115 of the chamber 110. The lid 90 is affixed to the container 80 and its chamber 110 and is moveable between a first open position relative to the open top 115 of the chamber 110 (as illustrated in FIGS. 4 and 5) and a second closed position relative to the open top 115 of the chamber 110 (as illustrated in FIGS. 6 and 7). In embodiments, the lid 90 is affixed to the chamber via a hinge 92. In embodiments, the chamber 110 and lid 90 are formed as a single integral injection molded plastic component, wherein the hinge 92 is a living hinge. The lid 90 thus allows selective covering of the cover 84 once it has been breached to prevent access to carrier particles and medicament particles remaining in the chamber 110 and/or to prevent the release or spillage of such particles remaining in the chamber 110 after use of the container 80 in a dry powder inhaler assembly. In the illustrated embodiment, the lid 90 has a ring 93 extending from an underside thereof, where the ring 93 is shaped to be received within and engage the open top 115 of the chamber 110. In other embodiments, the lid has no such ring and is generally flat along its underside, so that it can be folded over to selectively cover the cover 84 while the cover 84 is still intact. Suitable means such as a detent connection coupling, a friction fit, or an adhesive may be provided to permit retention of the lid 90 over the still intact cover 84.

As illustrated in FIGS. 4-7, the container 80 may include one or more alignment confirmation features such as a fin or vane 94 exposed on an outer surface of at least one portion of the container 80. The fin or vane 94 is shaped to conform to a respective receiving cavity or slot in a dry powder inhaler assembly, to ensure proper alignment of the container 80 therein for use.

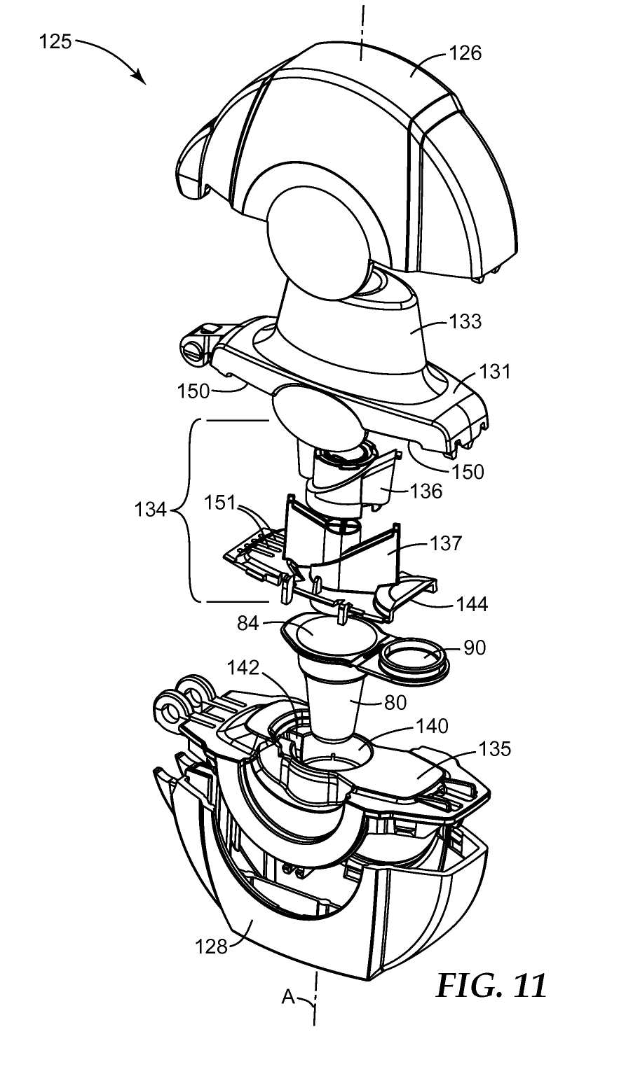

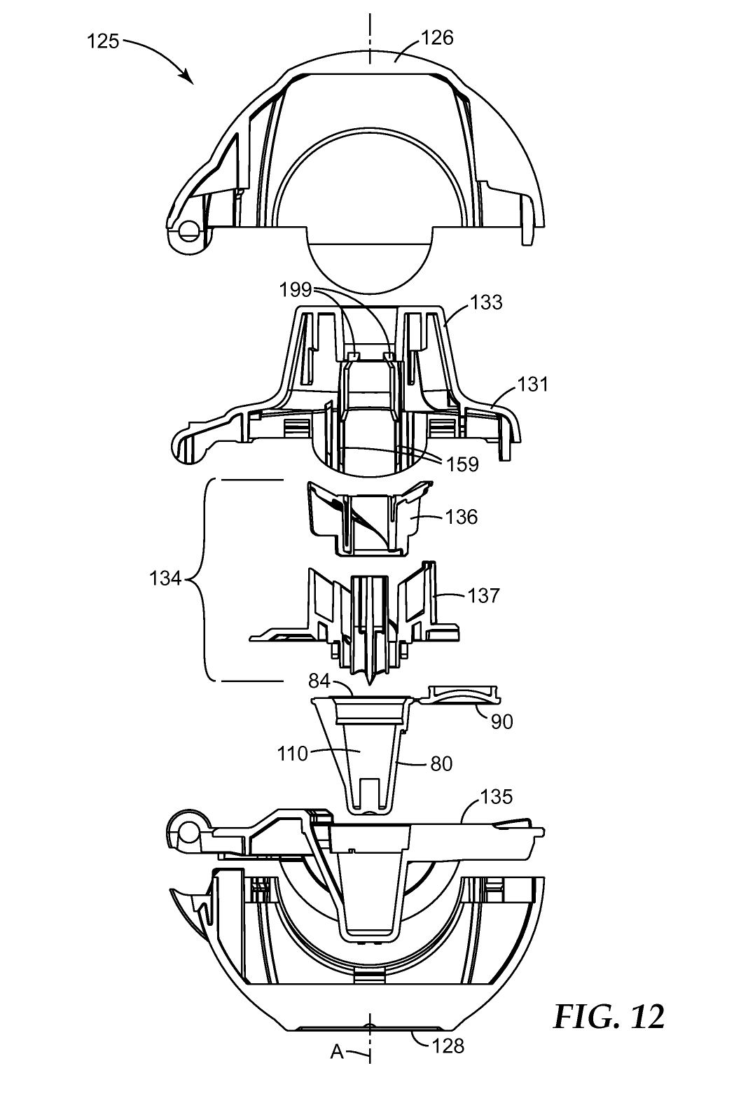

A dry powder inhaler assembly 125 is illustrated in FIGS. 8-10 in assembled form and in FIGS. 11-12 in exploded view form. The dry powder inhaler assembly 125 is depicted in FIG. 8 in its closed position for transport and storage. In this position, the dry powder inhaler assembly 125 typically does not contain a container of dry powder medicament particles. In its fully closed position depicted in FIG. 8, the dry powder inhaler assembly 125 is generally disc-shaped.

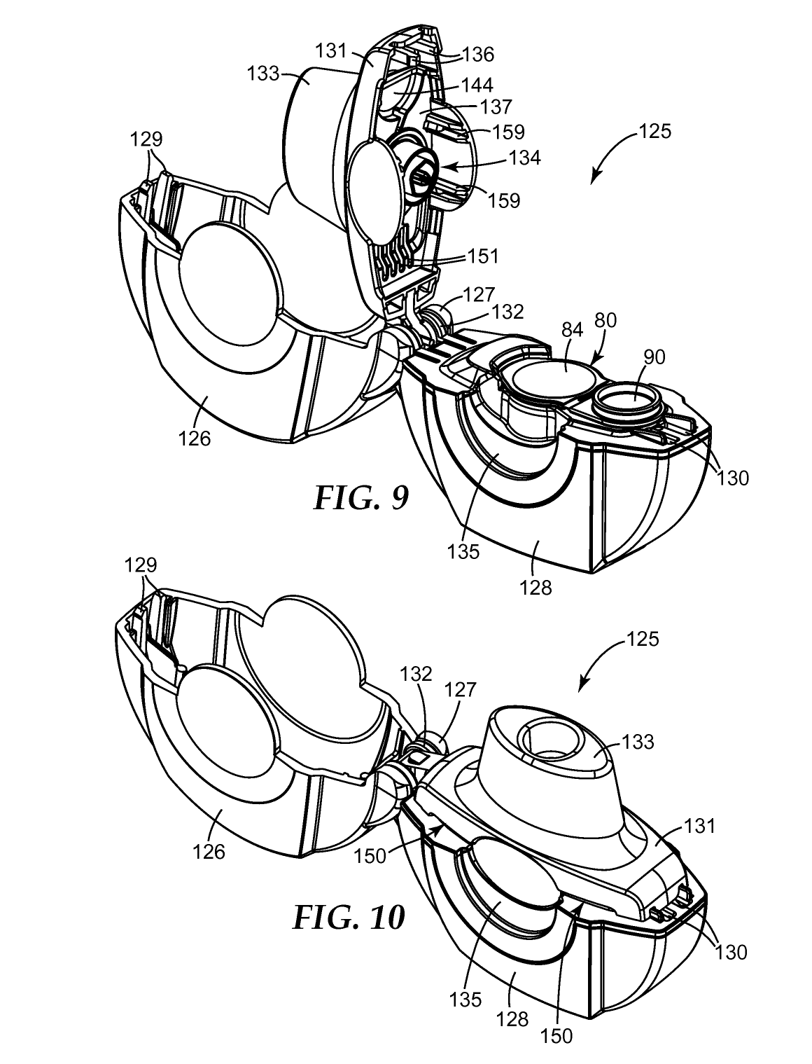

As illustrated in FIGS. 9 and 10, the dry powder inhaler assembly 125 has a generally “clamshell” configuration, with a clamshell cover 126 pivotally connected via a first hinge 127 to a lower member 128. When the clamshell cover 126 is pivoted about the first hinge 127 to engage the lower member 128, the dry powder inhaler assembly 125 is closed (as seen in FIG. 8). In a preferred embodiment, a mechanism is provided to retain those moveable elements in that closed position, such as, for example, a biasing spring, detent coupling connections, a friction fit, an adhesive, or the like. In embodiments, one or more male members 129 on one component (e.g., clamshell cover 126) are selectively and removably engaged with female members 130 that are coupled to the other component (e.g., lower member 128) for that purpose.

When the clamshell cover 126 is moved away from the lower member 128, another member, designated as upper member 131, is also then selectively movable away from the lower member 128, such as along a second hinge 132 (disposed coaxially with the first hinge 127). The upper member 131 includes a patient mouthpiece 133 on a top side thereof, and a medicament container piercing and air flow assembly 134 on a bottom side thereof.

As seen in FIG. 9, the lower member 128 has a body portion 135 coupled thereto that includes a receptacle shaped to receive and retain a disposable dry powder container 80 (such as previously illustrated in FIGS. 4-7). With the container 80 disposed within the body portion 135 of the lower member 128, the lower member 128 thus includes the chamber 110 of the container 80 therein. The dry powder medicament within the chamber 110 is sealably retained therein by means of the cover 84 on the container 80. As explained below, the medicament container piercing and air flow assembly 134 engages, pierces and moves aside the cover 84 to gain access to the dry powder medicament in the chamber 110 when the upper member 131 is pivoted to a closed position as illustrated in FIG. 10. Suitable means are provided for retaining the upper member 131 in its closed position relative to the lower member 128, such as, for example, a biasing spring, detent coupling connections, friction fit, an adhesive, or the like. In embodiments, one or more male members 136 on one component (e.g., upper member 131) are selectively and removably engaged with female members 130 that are coupled to the other component (e.g., lower member 128) for that purpose.

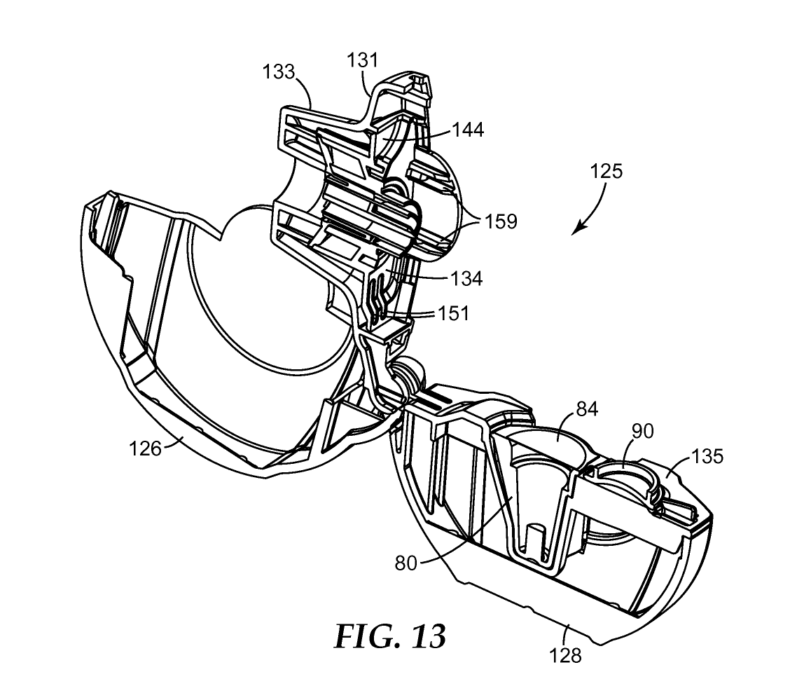

FIGS. 11 and 12 illustrate the components of the dry powder inhaler assembly 125, in views axially exploded along the axis A of the container 80. As illustrated in FIGS. 11 and 12, the medicament container piercing and air flow assembly 134 in embodiments is composed of two pieces, an upper airways component 136 and a lower airways component 137. In assembly, the upper airways component 136 is nested between the lower airways component 137 and the upper member 131, which two components are mutually secured by suitable engaging elements, such as detent elements or cooperatively engaging male and female elements, a friction fit, or via other suitable means such as adhesive. Upon assembly, the upper member 131 and medicament container piercing and air flow assembly 134 assume the assembled configuration illustrated in FIGS. 9 and 13. The upper member 131 and the medicament container piercing and air flow assembly 134 are secured together by suitable engaging elements, such as detent elements or cooperatively engaging male and female elements, a friction fit, or via other suitable means such as adhesive.

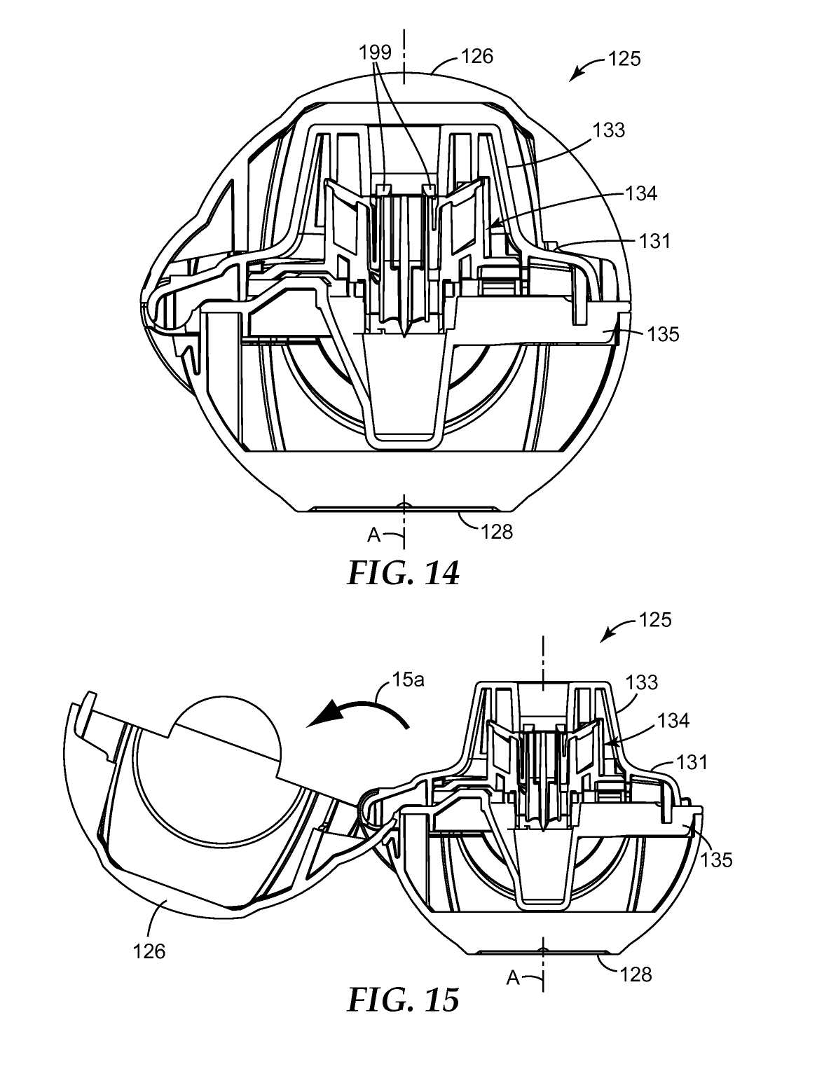

FIGS. 14-25 illustrate the dry powder inhaler assembly 125 of FIGS. 8-13, as its components may be configured during storage, container insertion/removal, and use. FIG. 14 is similar to FIG. 8, but depicts the dry powder inhaler assembly 125 in longitudinal section (an orientation also illustrated in FIGS. 15-25). In FIG. 14, the clamshell cover 126 is pivoted to its closed position atop the lower member 128 with the upper member 131 secured therebetween. No container of dry powder medicament particles is within the dry powder inhaler assembly 125 in FIG. 14 (depicting its configuration for storage and transport).

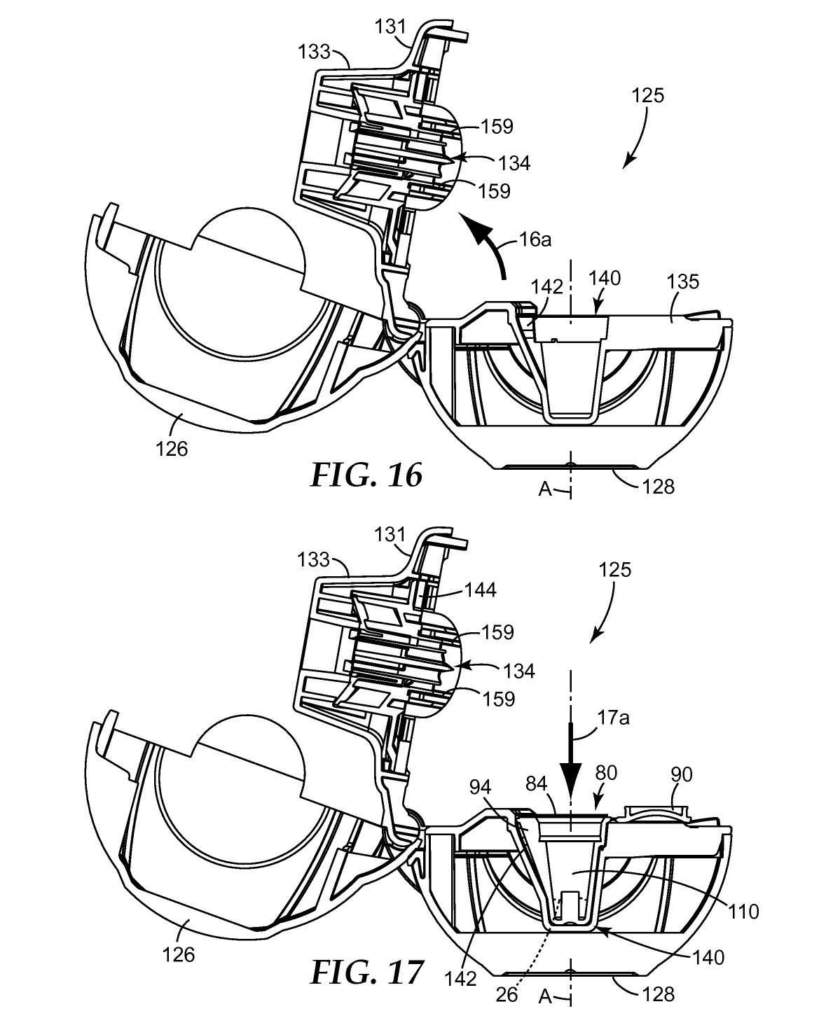

FIG. 15 illustrates the dry powder assembly 125 with the clamshell cover 126 moved to its open position, similar to the configuration illustrated in FIG. 10. The upper member 131 is still engaged with the lower member 128, but the mouthpiece 133 is now exposed. Arrow 15a illustrates pivotal movement of the clamshell cover 126 relative to the lower member 128. FIG. 16 depicts the dry powder inhaler assembly 125 in a configuration similar to that depicted in FIG. 9, although without the presence of the container 80, with the upper member 131 disengaged at one end from the lower member 128 and moved to an open position relative thereto, as illustrated by arrow 16a. This fully exposes a receptacle 140 in the body portion 135, along with an associated container alignment slot 142 extending radially therefrom, relative to axis A (a portion of slot 142 is also seen in FIG. 11).

In FIG. 17, a container 80 has been inserted into the receptacle 140 (generally in the axial direction indicated by arrow 17a), with the vane 94 on the container 80 being received within the slot 142 adjacent the receptacle 140. This aligns the container 80 relative to the dry powder inhaler assembly 125 for use. This also aligns the lid 90 of the container 80 within the assembly 125 so that it is accommodated by a recess 144 on a bottom portion of the lower airways component 137 of the medicament container piercing and air flow assembly 134 (see FIG. 9).

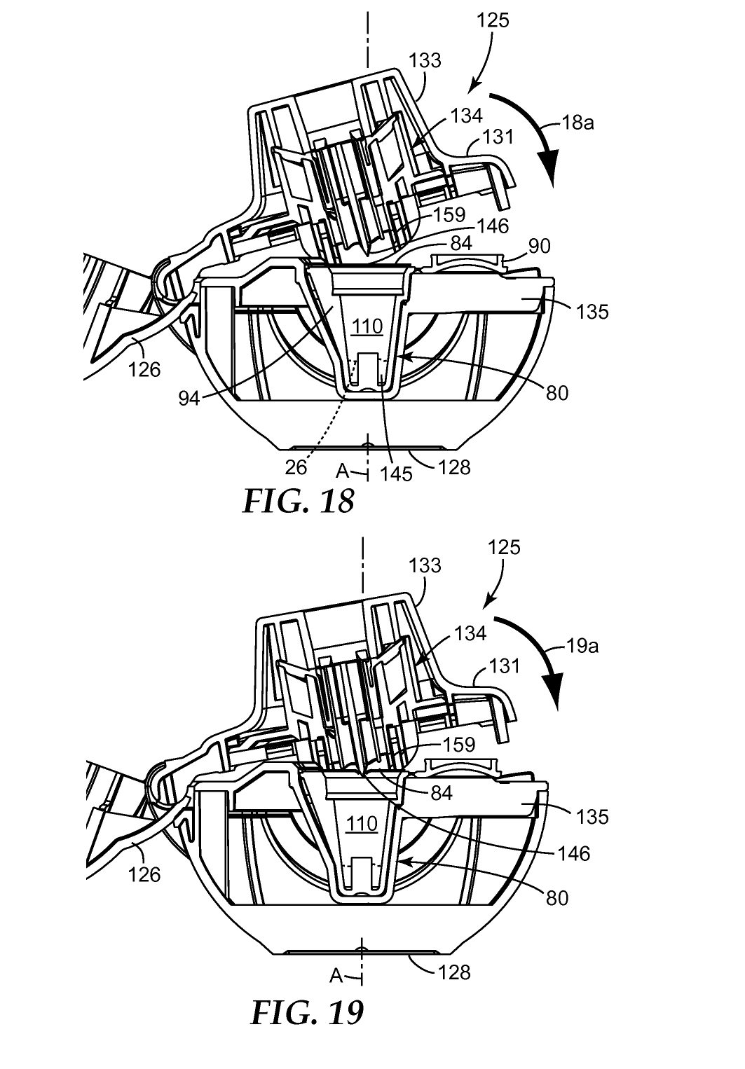

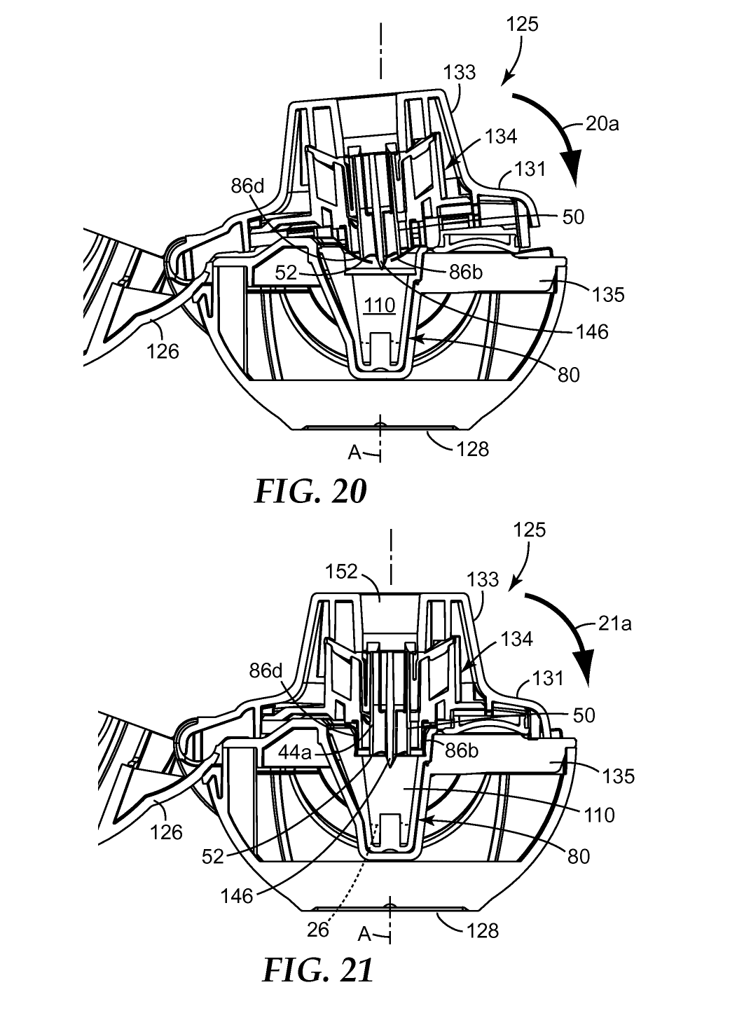

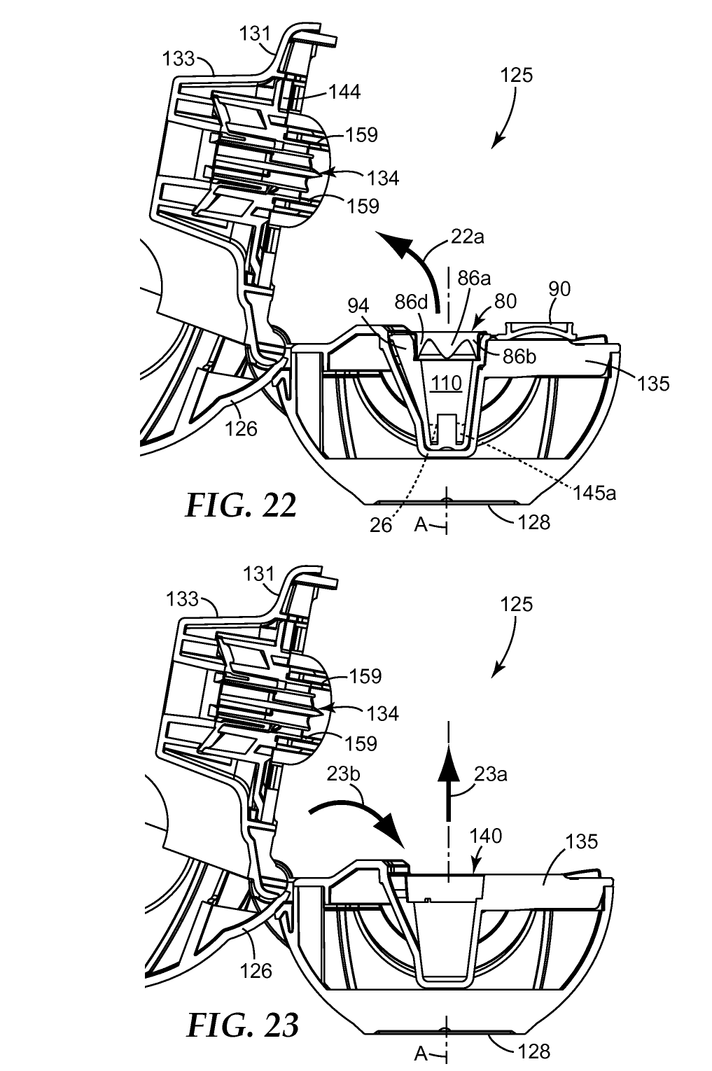

Once the container 80 has been properly seated in the receptacle 140, the dry powder inhaler assembly 125 is ready to have its upper member 131 moved into its closed position relative to its lower member 128, thereby breaching the cover 84 on the container 80, and placing the assembly 125 in condition for use by a patient for inhalation of the dry powder medicament particles 145 (FIG. 18) therein (which, as noted above, may be intermingled with carrier particles as well in the chamber 110 prior to use). FIGS. 18, 19, 20 and 21 illustrate the sequence of cover 84 engagement, breach and capture via movement of the upper member 131 relative to the lower member 128 (although in those FIGS., and also in FIGS. 22 and 23, the clamshell cover 126 is only partially depicted). Specifically, a cover piercing element 146 of the lower airways component 137 has a leading portion thereof that first contacts the cover 84 at its center (i.e., along axis A, as illustrated in FIG. 19). As penetration continues via movement of the upper member 131 downwardly relative to the lower member 128, the cover piercing element 146 pushes through the cover 84, and the first end 52 of the outlet tube 50 of the lower airways component 137 engages separated portions of the cover 84, as seen in FIG. 20. This aids in pushing those separated portions of the cover 84 against the inner wall surface of the container 80, thereby clearing possible obstructions from air flow in and out of the chamber 110 of the container 80. The separated portions of the cover 84 (designated in FIG. 21 as flaps 86d and 86b) are captured between an inner circumferential surface of the chamber 110 (and specifically its third upper portion thereof) and a cylindrical outer surface of a wall 44a of the annular section 44 that in part defines one or more air inlet channels, and that is also born by the lower airways component 137. The motion of the upper member 131 relative to the lower member 128 in FIGS. 18-21 is depicted by arrows 18a-21a, respectively.

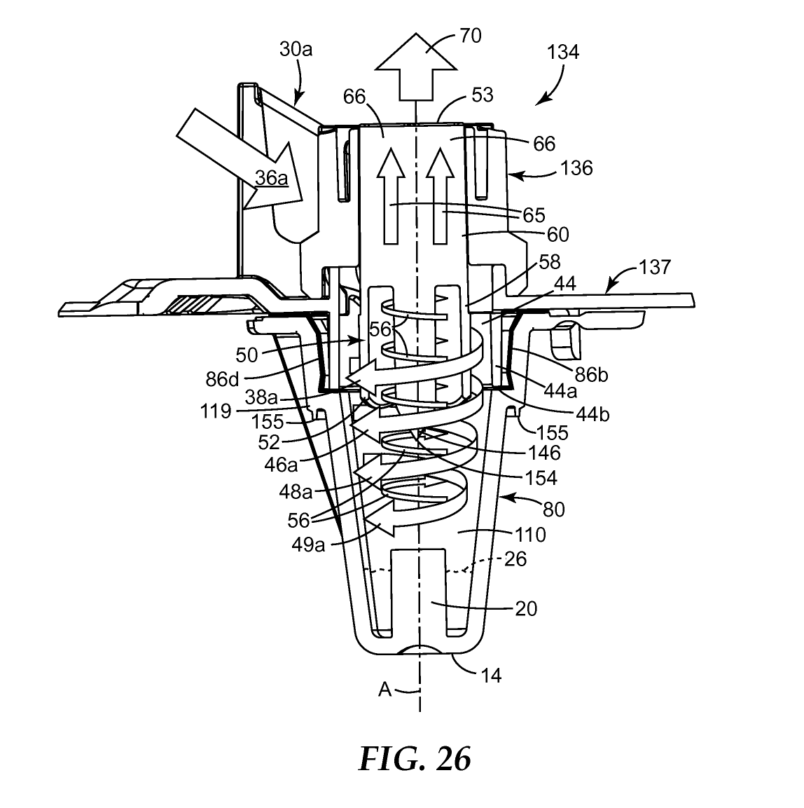

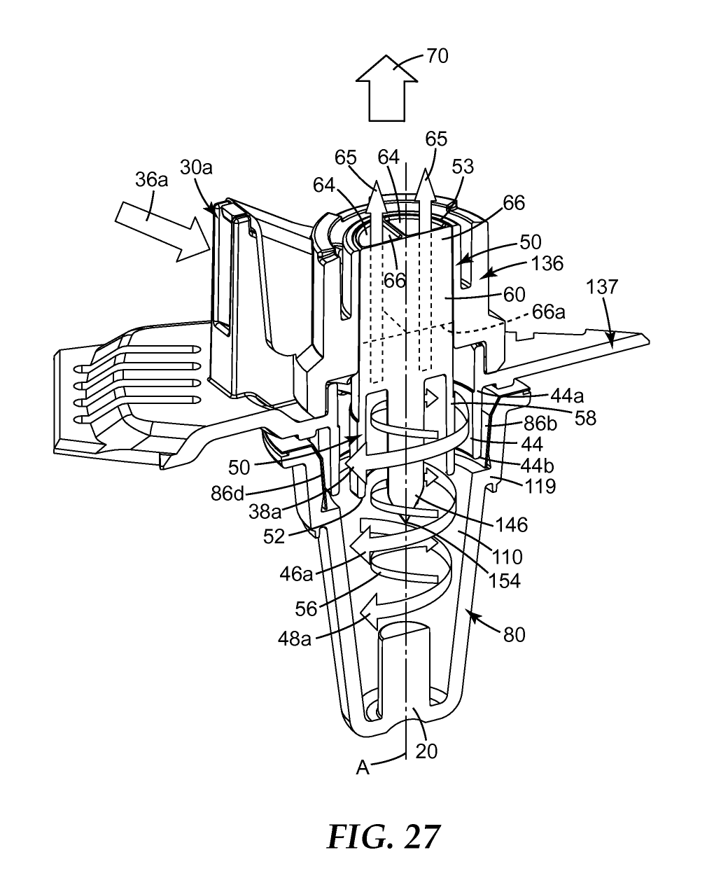

Once the dry powder inhaler assembly 125 has been placed in the configuration illustrated in FIG. 21, it is ready for use by a patient for inhalation of the dry powder medicament therein. This occurs by means of the air flow discussed above and illustrated with respect to FIGS. 1-3B. In part, such air flow and a schematic representation of some of the operable components illustrated in FIG. 21 are depicted in FIGS. 26 and 27. Like reference numerals are used in the illustrations of FIGS. 26 and 27 relative to the illustrations of FIGS. 1-3B, to indicate the commonality of structure and air flow through the structure. For clarity of illustration, air flow from only one air inlet channel 30a is illustrated in FIGS. 26 and 27. A dry powder inhaler assembly of the present disclosure may be constructed with only one air inlet channel if desired. However, the dry powder inhaler assembly 125 of FIG. 21 has two symmetrically opposed and shaped air inlet channels (such as illustrated schematically in FIGS. 1-3B).

The upper member 131 is provided with openings, or alternatively is provided with cooperative cutouts adjacent the lower member 128 or its body portion 135, as indicated by cutout openings 150 seen in FIGS. 10 and 11. Such openings 150 are provided on both sides of the upper member 131.

Upon use, a patient places his or her lips around the mouthpiece 133 and inhales. Ambient air is then drawn into the upper member 131 through the openings 150, through internal air flow openings 151 therein (see FIG. 9), and then into open ends of the air inlet channels 30a, 30b, as indicated by air flow arrows 36a and 36b in FIGS. 1, 2, 3A, 26 and 27. Such air flow is drawn (via the unique formation of the air inlet channels) into a free vortex within the chamber 110 of the container 80, as illustrated by air flow arrows 38a, 38b, 46a, 46b, 48a, 48b and 49a in FIGS. 2, 26 and 27. As explained above, the air flow stream from one air inlet channel is disposed in a stratified way with the air flow stream from an adjacent air inlet channel, as illustrated in FIG. 2 (such stratification is not depicted in FIGS. 26 and 27, for clarity of illustration).

The free vortex formed by the downward spiral of air in the chamber 110 is interrupted and reversed by a combination of the bottom wall 14 and the flow disruptor 20 within the chamber 110. As a result, a forced vortex illustrated by air flow arrows 56 spirals upwardly within the free vortex and, to the extent allowed by the size of the outlet tube 50, continues upwardly within the first section 58 of the outlet tube 50. At some point along the interior of the outlet tube 50, the forced vortex encounters one or more divider walls 66 which interrupts its helical flow upwardly through the outlet tube 50 and converts such flow into an air flow in a direction generally parallel to the axis A, as illustrated by air flow arrows 65. The air in the outlet tube 50 flows through one or more ducts 64 defined in part by the divider walls 66 within the second section 60 of the outlet tube 50. In embodiments, the divider walls 66 and associated ducts 64 extend entirely along the second section 60 of the outlet tube 50, to its second end 53 (see FIG. 27). In an alternative embodiment, illustrated by dashed line edges 66a in FIG. 27, the divider walls 66 extend only along a portion of the second section 60 of the outlet tube 50. In addition, while each of the divider walls 66 is depicted as generally linear and continuous, alternative configurations are contemplated. For example, adjacent walls may be staggered as disposed along the second section of the outlet tube, or one or more of the divider walls may have, at least in part, a curved or helical surface for air flow diversion (or disruption) therealong. Further, while four ducts 64 are illustrated in the figures, any number of ducts in the second section 60 of the outlet tube 50 will suffice, so long as the helical flow of the forced vortex (represented by air flow arrows 56) is interrupted and converted into flow that runs generally parallel to the axis A by the time it reaches the second end 53 of the outlet tube 50 (or is at least significantly reduced in its spiraling energy and significantly converted into motion along a generally axially aligned direction). Air flow exiting the second end 53 of the outlet tube 50 is represented by air flow arrow 70, and enters a conduit 152 (see FIG. 21) within the mouthpiece 133. The conduit 152 opens directly into the patient's mouth when the dry powder inhaler assembly 125 is used, and is generally coaxially aligned with the axis A of the chamber 110. After use, the bulk of the larger carrier particles remain in the chamber 110 for disposal with the used container 80.

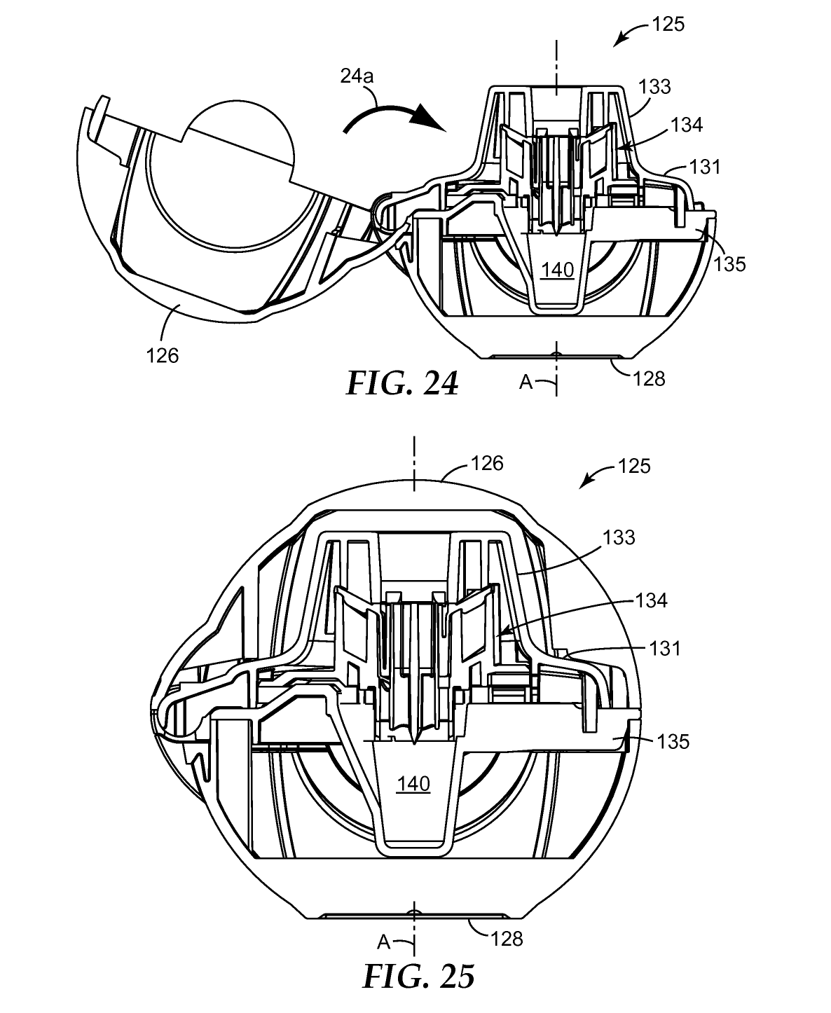

The container 80 is accessed and removed from the dry powder inhaler assembly 125 by reversing the steps of its insertion. First, as illustrated in FIG. 22, the upper member 131 is moved away from the lower member 128, thereby exposing the used container 80 with its breached cover 84 (depicted by way of representation by cover flaps 86a, 86b and 86d). The upper member 131 is moved relative to the lower member 128 such as via pivoting in direction of arrow 22a. When the container 80 is in the form illustrated in FIGS. 5-7, its lid 90 is laid in its open position next to the container during use of the dry powder inhaler assembly 125. In order to prevent access to or spillage of the remaining particles within the container 80, a user may now close the lid 90 over the chamber 110, thereby resealing the chamber of the container. The used container 80 is then removed from the receptacle 140 (such as in direction of movement arrow 23a in FIG. 23), thereby making receptacle 140 available for insertion of another full container 80 if desired. If a patient's dosage of medicament is complete, however, after removal of the used container 80, the upper member 131 may be moved back into an engaged and mated position with the lower member 128 (such as in direction of arrow 23b in FIG. 23 and arrow 24a in FIG. 24) to again assume the configuration illustrated in FIG. 15. To complete the closure process and prepare the dry powder inhaler assembly 125 for transport and/or storage, the clamshell cover 126 is likewise moved back into engagement with the lower member 128, thereby encasing the upper member 131 therebetween (as illustrated in FIG. 25) placing the dry powder inhaler assembly 125 again in the configuration illustrated in FIG. 14.

As is readily evident from the sequence of component movement in FIGS. 14-25, the upper member 131 has a first mating surface, a first air inlet and a first air outlet. The lower member 128 has a second mating surface adapted for selective engagement with the first mating surface, and holds the chamber 110 formed at least in part for reverse cyclone flow therein. The upper and lower members 131 and 128 are moveable relative to one another between a first position wherein the mating surfaces are spaced apart (see, e.g., FIGS. 9, 13, 16, 17, 18, 19, 20, 22 and 23) and a second position wherein the mating surfaces are engaged (see, e.g., FIGS. 10, 14, 15, 21, 24, 25, 26 and 27). The respective mating surfaces on the upper and lower members 131 and 128 may be any surfaces on those components that are spaced apart when in the first position and then somehow engaged (i.e., in contact) when in the second position (including, e.g., with respect to the lower member 128, one or more surfaces of the container 80 retained within the lower member 128). When so engaged, portions of each of the first air inlet (i.e., annular section 44 thereof) and air outlet (i.e., a portion of the first section 58 of outlet tube 50) are within the chamber 110 of the upper member 131. The upper member 131 includes the cover piercing element 146, with at least a portion of the cover piercing element 146 disposed within the chamber 110 of the lower member 128 when the upper and lower members 131 and 128 are in the second position (e.g., as illustrated in FIGS. 26 and 27).

In embodiments, the upper member 131 and lower member 128 move relative to one another such that a leading portion (e.g., a tip 154 of the cover piercing element 146; see FIGS. 26 and 27) lies at the center line (i.e., axis A) of the chamber 110 when the upper and lower members 131 and 128 are in their second position. In embodiments, the tip 154 of the cover piercing element 146 first contacts the cover 84 at the center of the cover 84 (i.e., along axis A).

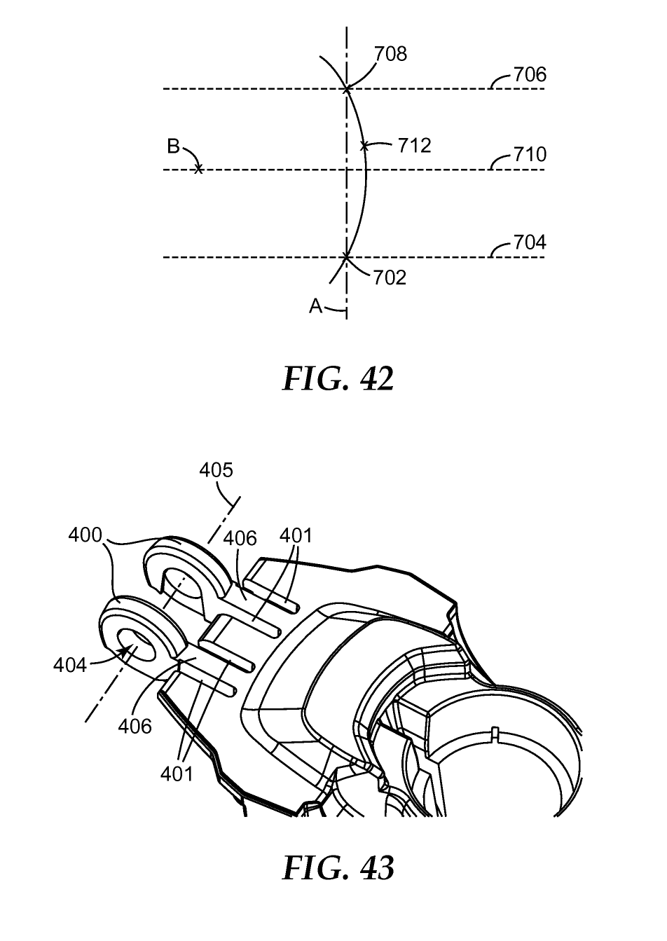

This arrangement is illustrated schematically by FIG. 42, where A is the axis of the chamber of the inhaler assembly and B represents an axis of the hinge between the upper member 131 and lower member 128, extending perpendicular to the axis A. Arc 700 illustrates the path traversed by the tip 154 of the cover piercing element 146 as it moves downwardly (during piercing of cover 84) to its final position 702 when the upper and lower members 131 and 128 are in their second position (i.e., when the cover piercing element 146 is within the chamber 110 of the container 80, and the assembly 125 is ready for inhalation). In this position, the tip 154 is disposed on the axis A. A plane normal to the axis A at this point is illustrated as plane 704 in FIG. 42. A plane 706, also normal to the axis A, illustrates the top surface of the cover 84. As depicted in FIG. 42, the arc 700 traversed by the tip 154 first engages the cover 84 at point 708 which is also disposed along the axis A. A plane 710 is also normal to the axis A, and includes the axis B. The plane 710 is midway between the planes 704 and 706. The hinge axis B and associated moveable components are related to the axis A such that the arc 700 traversed by the tip 154 intersects the axis A when the tip 154 engages the cover 84 (at plane 706) and when the tip 154 is in its fully inserted position into the chamber 110 of the container 80 (as at plane 704). This condition is met even though the tip 154 is not aligned along the axis A when traversing other parts of the arc 700 (such as, e.g., at point 712 along the arc 700). In this regard, the leading portion or tip 154 of the cover piercing element 146 first contacts the cover 84 when the upper member 131 is in a third position relative to the lower member 128. That third position is disposed between the first and second positions, wherein the upper member 131 and the lower member 128 pivot relative to one another between the first, third and second positions, respectively. Such pivoting occurs about the hinge axis B (FIG. 42) that is positioned at a height that is approximately midway between the height of the tip 154 of the cover piercing element 146 when the upper member 131 is in the third position (illustrated by point 708 in FIG. 42) and the height of the tip 154 of the cover piercing element 146 when the upper member 131 is in the second position (illustrated by point 702 in FIG. 42), such heights all being measured parallel to the axis A.

In embodiments, the upper member includes a second air inlet (such as air inlet channel 30b), and a portion of the second air inlet (again, annular section 44 thereof) is within the chamber 110 of the lower member 128 when the upper and lower members 131 and 128 are in the second position (such as illustrated in FIG. 21).

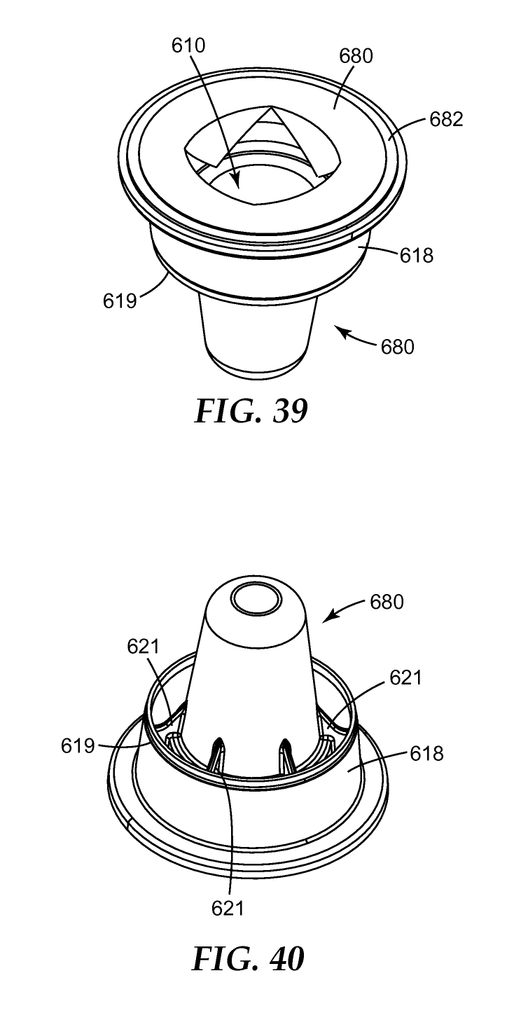

While the receptacle 140 is shaped to receive the container 80, its configuration may also be shaped and arranged such that containers of alternative internal size (i.e., different chamber sizes) can be suitably received and retained within the receptacle 140 (see, e.g., FIGS. 39-41C, as further discussed below). Regardless of its size, each container received within a receptacle includes a cup segment and a cover sealed thereover.

As illustrated in FIGS. 26 and 27, the annular shoulder 119 of the container 80 is formed to selectively mate with a lower end of the air inlet channel(s) (i.e., with annular section 44). The annular section 44 of the air inlet channel(s) is in part defined by an outer cylindrical wall 44a that has a lower end 44b. As best seen in FIG. 26, the first end 52 of the outlet tube 50 extends further into the chamber 110 than the first end 44b of the air inlet channel(s) represented by annular section 44.

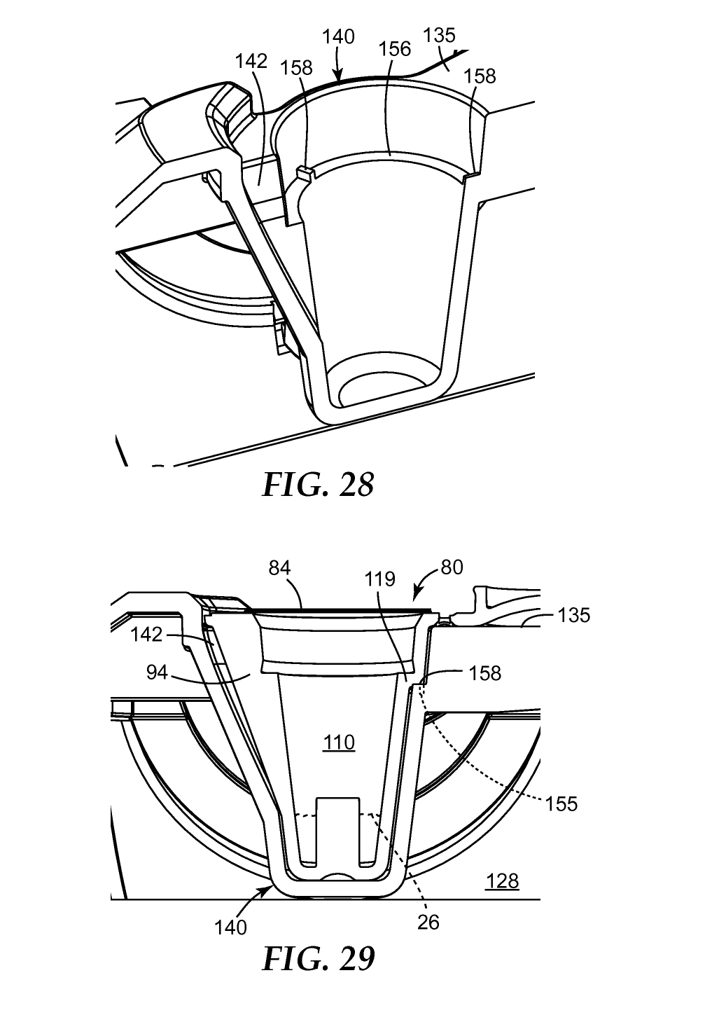

In embodiments, the annular shoulder 119 of the container 80 has, on an outer surface thereof, an axially extending projection 155, as depicted in FIG. 7. The projection 155 may be continuous around the annular shoulder 119 (i.e., the projection 155 may be annular), or it may be discontinuous. In embodiments, the projection 155 is defined as an annular fin. The enclosure 140 is likewise formed with an annular, radially extending shoulder, such as annular shoulder 156 illustrated in FIGS. 28 and 29. One or more projections or tabs 158 are disposed on the annular shoulder 156 of the receptacle 140 (such as illustrated in FIGS. 28 and 29). When the container 80 is disposed within the receptacle 140, the projection 155 thereon engages an upper surface of each tab 158. The material forming the projection 155 is deformable, so that upon pressure downwardly against the container 80 (e.g., when the dry powder inhaler assembly 125 is placed in its closed position such as illustrated in FIG. 21 for use) the projection 155 deforms against and over each of the tabs 158, thereby creating a biasing force urging the container 80 upwardly in an axial direction relative to the receptacle 140, and against those portions of the medicament container piercing and air flow assembly 134 that engage the container 80 and extend at least partly into the chamber 110 thereof.

As can be appreciated by the above discussion, it is important that the inlet air channels and outlet tube be placed in a known position relative to the reverse cyclonic flow chamber. In addition to those already depicted and discussed, one means for additionally implementing such alignment is the use of guiding ribs disposed on inner surfaces of the upper member 131. Such guiding ribs are depicted in FIGS. 9, 12, 16-20, 22 and 23, as ribs 159. Each rib 159 has a face aligned circumferentially relative to the axis A for use in engaging and guiding the upper member 131 downwardly about the container 80 (and specifically about an outer perimeter edge of the container's rim surface 82) as the upper member 131 moves to its second position relative to the lower member 128. Such additional guidance for alignment of the medicament container piercing and air flow assembly 134 relative to the container 80 may take many forms, and may include guidance features on the container itself, on the assembly 134, or on a separate part of the assembly 125.

In the embodiment of the dry power inhaler assembly illustrated in FIGS. 8-25, the clamshell cover 126, lower member 128 and upper member 131 are pivotally connected via coaxial hinges 127 and 132. This hinge arrangement is facilitated by the following features, as illustrated in FIGS. 43-47 of the hinges 127, 132 of the dry powder inhaler assembly 125.

As best seen in FIG. 43, the body portion 135 has a pair of protruding eyelets 400 spaced apart along a hinge axis 405. Each eyelet has an elongated stalk 406 by means of which it is attached to the main part of the body portion 135. Slots 401 are provided between stalks 406 in order that the stalks have a sufficient length to offer flexibility of movement to the protruding eyelets and stalks. In particular, the eyelets 400 and stalks 406 have a small lateral thickness (i.e., in a direction parallel to the hinge axis 405) relative to their combined length as measured from the inner ends of the slots 401 to the furthest protruding ends of the eyelets 400. The lateral thickness and the length of the slots 401 are suitably chosen with regard to the physical properties of the material of the body portion 135. Typically an injection molded polymer material (e.g., polypropylene) would be used for body portion 135. The aspect ratios illustrated, with a combined eyelet and stalk length of approximately 13 mm, are typically suitable for such a material.

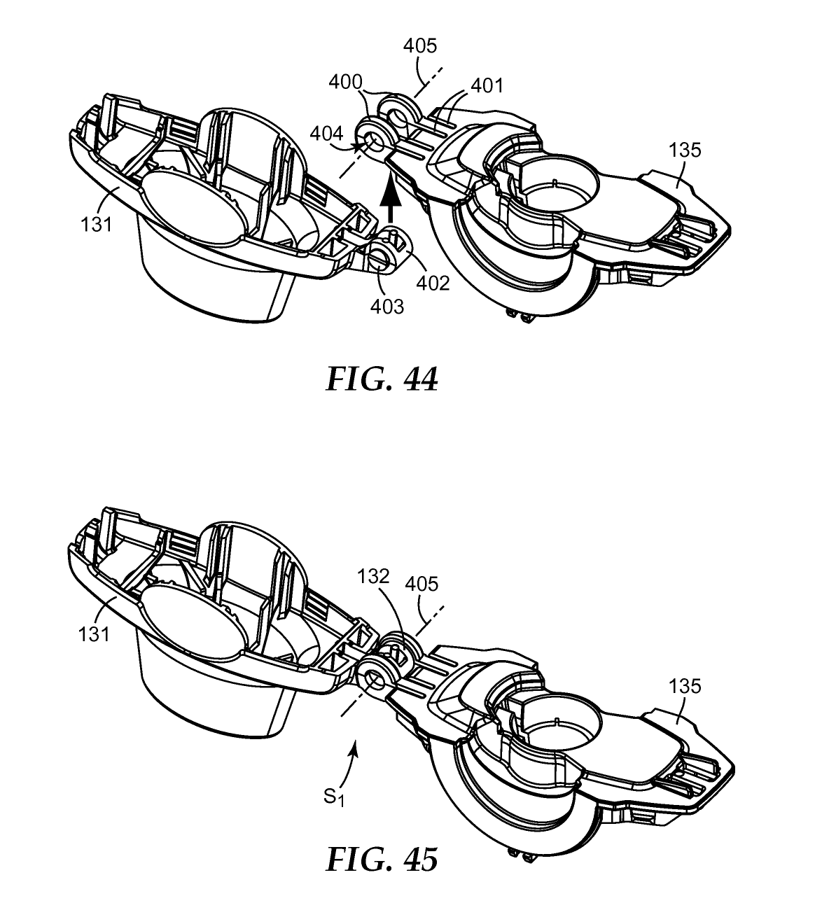

The length and flexibility of the eyelets and stalks, by virtue of the features just described, allows a boss 402 on the dry powder inhaler upper member 131 to push easily between the two eyelets 400 during inhaler device assembly (See FIG. 44). As the boss 402 interacts with the eyelets 400, the stalks 406 and eyelets 400 deflect sideways (outwards, away from one another as a pair) along the general direction of the hinge axis 405. This allows a central axle feature 403 on each side of the boss 402 to snap into an aperture 404 at the center of each eyelet 400. As each axle feature 403 locates into its corresponding aperture 404, the eyelets 400 and stalks 406 are free to return elastically to their initial positions (i.e., straight out from body portion 135 in a direction orthogonal to the hinge axis 405). The large measure of outward (i.e. away from one another as a pair, generally along the hinge axis 405) flexibility conferred on the eyelets 400 by the slots 401 allows axle features 403 of substantial and robust size to be used, with a good degree of dimensional overlap with the eyelets 400 along the line of the hinge axis 405. As so assembled via hinge 132, upper member 131 and body portion 135 now form a sub-assembly S₁ as depicted in FIG. 45.

The clamshell cover 126 can be assembled onto the outer sides of the eyelets 400 at this stage, or can be added as the final step in the assembly of this hinge arrangement. The clamshell cover 126 has axle features mounted on arms of a substantial length and flexibility, to allow adequate engagement with the outer sides of the eyelets 400 of the body portion 135.

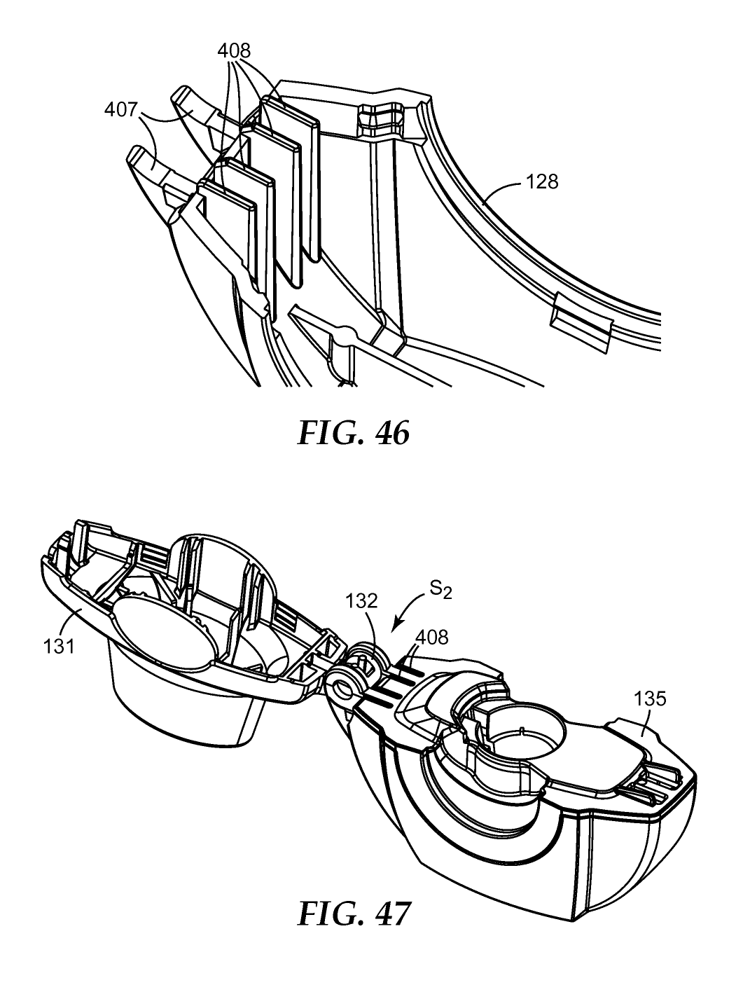

Either before or after assembly of the clamshell cover 126, the lower member 128 is assembled on to the sub-assembly S₁ depicted in FIG. 45. As depicted in FIG. 46, the lower member 128 has two large ribs 407 that mate against the underside of the eyelets 400 of the body portion 135 after assembly. It also has a plurality (e.g., four) smaller protruding ribs 408 that fit into respective slots 401 in the body portion 135 when the lower member 128 is assembled onto the sub-assembly S₁ of FIG. 45. As the four ribs 408 enter the slots 401 they effectively lock the stalks 406 and eyelets 400 in position laterally, i.e., preventing significant deflection generally along the direction of the hinge axis 405. When lower member 128 is assembled onto the body portion 135 and the upper member 131, as depicted in FIG. 47, the hinge assembly loses its lateral flexibility, thus conferring important dimensional and positional rigidity onto a device sub-assembly S₂, seen in FIG. 47. In short, this hinge arrangement allows flexibility during a first stage of assembly and then confers rigidity after a second stage of assembly.

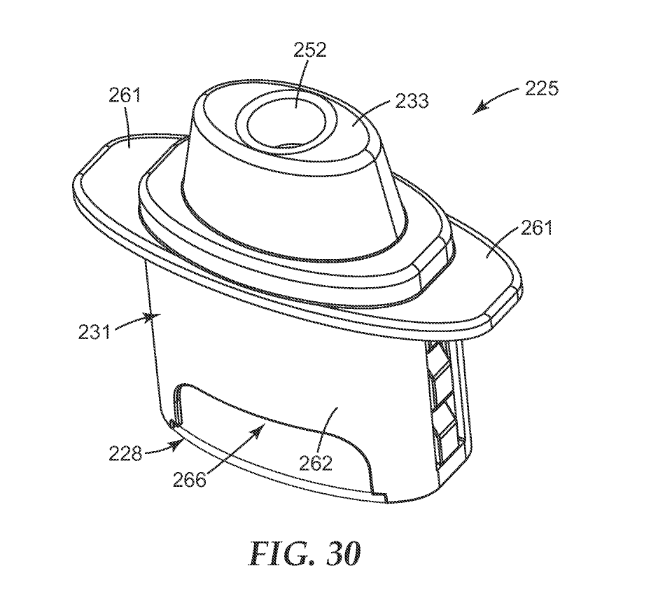

FIGS. 30-33 illustrate an alternative embodiment for a dry powder inhaler assembly 225 of the present disclosure. The dry powder inhaler assembly 125 illustrated in the previously described embodiment was designed for use with a replaceable container 80 for the dry powder medicament particles. The dry powder inhaler assembly 225 is designed as a single use inhaler. Once the dry powder medicament particles inside the assembly 225 are accessed in use, the entire assembly 225 is disposed of, and no portion thereof is reused. In other words, there is no replaceable container associated with the assembly 225.

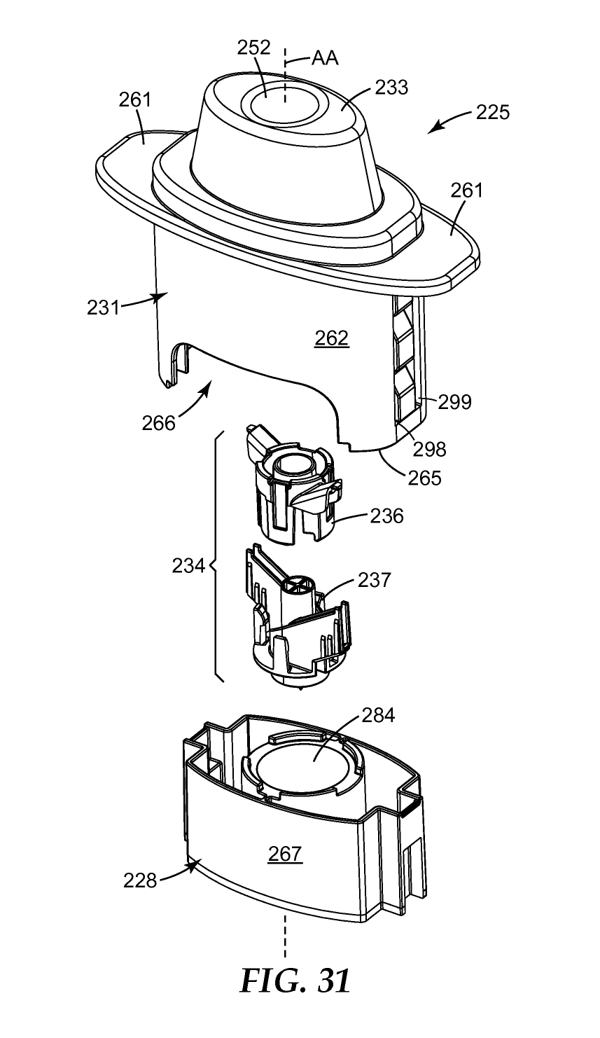

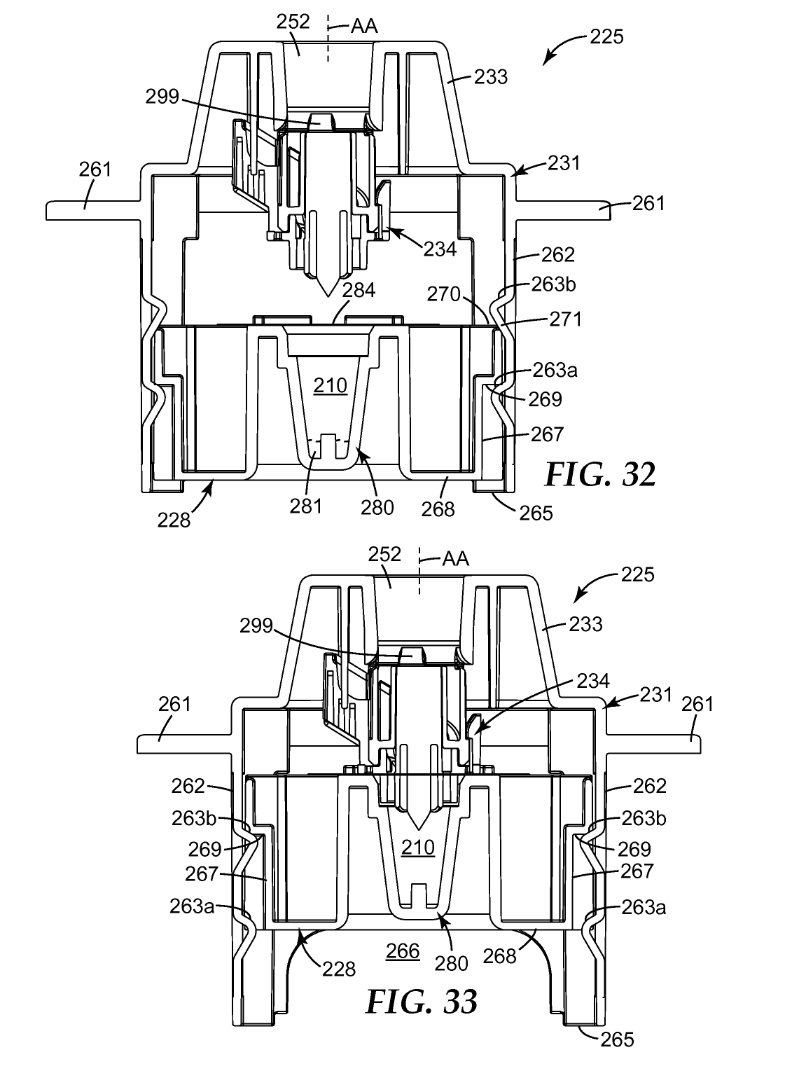

The dry powder inhaler assembly 225 has a first upper member 231 and a second lower member 228. The upper member 231 has a mouthpiece 233 suitable for use by a patient via insertion into the patient's mouth for inhalation. The mouthpiece 233 likewise includes a conduit 252 which is disposed coaxially about a central axis AA of the assembly 225. The upper member 231 has a radially projecting flange 261 to facilitate handling of the dry powder inhaler assembly 225 during use. The upper member 231 has an axially extending wall 262 which on at least opposite sides thereof, has a first pair of lower inner shoulders 263a and a second pair of upper inner shoulders 263b (see FIGS. 32-33), with vertical slots 298, 299 running on either side of them (see FIG. 31). These two pairs of slots, one pair at each opposite side of axially extending walls 262, confer flexibility on the central portions of the wall, allowing the inner shoulders 263a and 263b to flex outwardly when required. Along a bottom edge 265 of the wall 262, the upper member has opposed upwardly extending cut out 266, to facilitate operation of the dry powder inhaler assembly 225, as explained below.

The lower member 228 is defined by an axially extending enclosure having an upright wall 267, with bottom wall surfaces 268 (see FIGS. 32-33). Lower member 228 also includes a container 280 formed therein and aligned coaxially with respect to the axis AA. The container 280 has an interior form like that of container 80 and, as depicted, has its open top covered by a pierceable layer of material to form a cover 284 thereon. The container 280 thus defines a chamber 210 therein formed to facilitate reverse cyclone air flow for stirring and separation of dry powder medicament and carrier particles sealed therein, such as illustrated by medicament particles 281 in FIG. 32.

The wall 267 of the lower member 228 has, on opposed sides thereof (and in alignment with the opposed sides of the wall 262 of the upper member 231) downward facing edge portions 269 aligned to selectively engage either the lower shoulder 263a or upper shoulder 263b of the wall 262. In FIG. 32, the edge portions 269 are depicted engaged with the lower shoulders 263a, while in FIG. 33, the edge portions 269 are depicted engaged with the upper shoulder 263b. The portions of the walls 262 that bear the shoulders 263a and 263b are sufficiently resilient to allow axial movement of the lower member 228 relative to the upper member 231 between a first open position (as seen in FIG. 32) and a second closed position (as seen in FIG. 33). In the open position, each edge portion 269 engages a respective lower shoulder 263a. In the closed position, each edge portion 269 engages a respective upper shoulder 263b. Wall 267 of the lower portion 228 has an upper edge 270 that, when in its open position (as seen in FIG. 32), engages a ramp 271 adjacent each upper shoulder 263b. The lower member 228 is thus retained in the open position by an interference fit between those elements. However, when the lower member 228 is pushed upwardly relative to the upper member 231, the upper edge 270 slides over the ramp 271 by deformation of the central portions between slots 298 and 299 of both opposite sides of wall 262. This deformation allows the lower inner shoulders 263a to move outwardly. This allows relative sliding of the lower and upper members 128 and 131, as they translate axially relative to one another. The cut-outs 266 and flanges 261 on the upper member 131 facilitate user handling manipulation of the moveable lower and upper members 128 and 131 during use.

As illustrated in FIGS. 31-33, a medicament chamber piercing and air flow assembly 234 is mounted (by suitable engagement, such as detent couplings, male and female fittings, a friction fit, adhesive bonding or the like) to an interior surface of the upper member 231. The medicament chamber piercing and air flow assembly 234 in embodiments is composed of two pieces, an upper airways component 236 and a lower airways component 237. In assembly, the upper airways component 236 is nested within and secured to the lower airways component 237 by suitable engaging elements, such as detent elements, cooperatively engaging male and female elements, a friction fit, or via other suitable means such as adhesive. Upon assembly, the upper member 231 and medicament chamber piecing and air flow assembly 234 assume the assembled configuration illustrated in FIGS. 32-33.

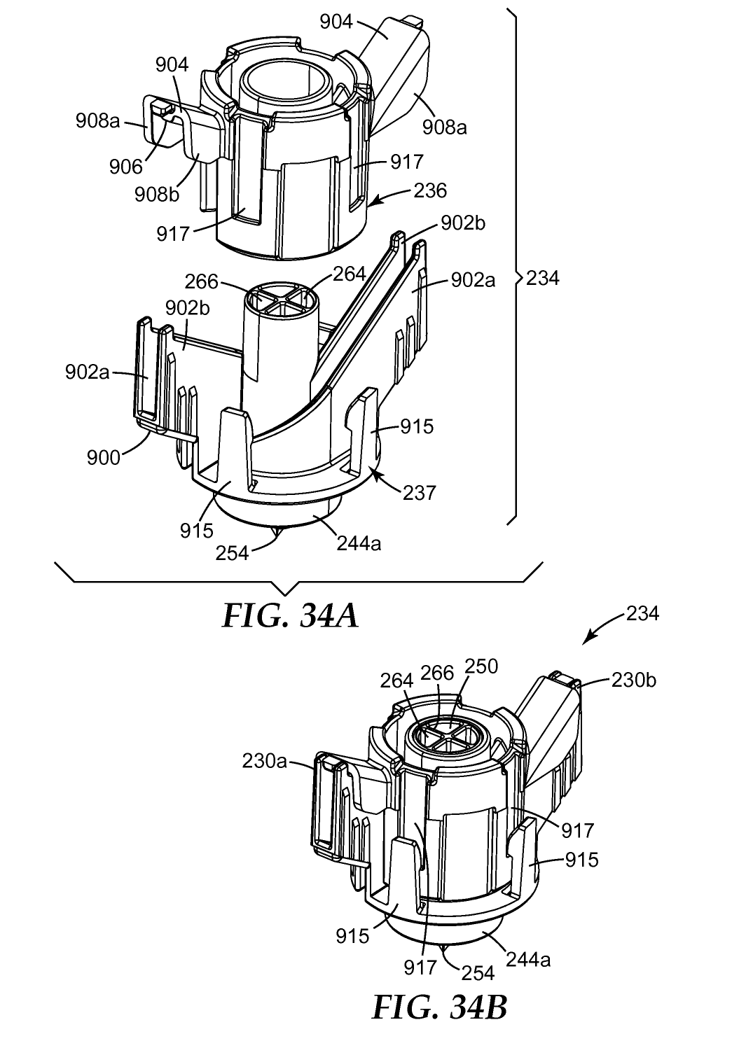

Functionally, the medicament chamber piecing and air flow assembly 234 operates in a manner similar to that of the medicament container piercing and air flow assembly 134 illustrated in FIGS. 9 and 11-29. As explained in more detail below with respect to FIGS. 34A-38C, the medicament chamber piercing and air flow assembly 234 has at least one air inlet and an air outlet, and includes a cover piercing arrangement for engaging, piercing and separating the cover 284 over the chamber 210. FIG. 33 illustrates the medicament chamber piercing and air flow assembly 234 in position for use with respect to the chamber 210, where portions of the assembly 234 (including at least a portion of an air inlet, air outlet and piercing element) are disposed within the chamber 210.

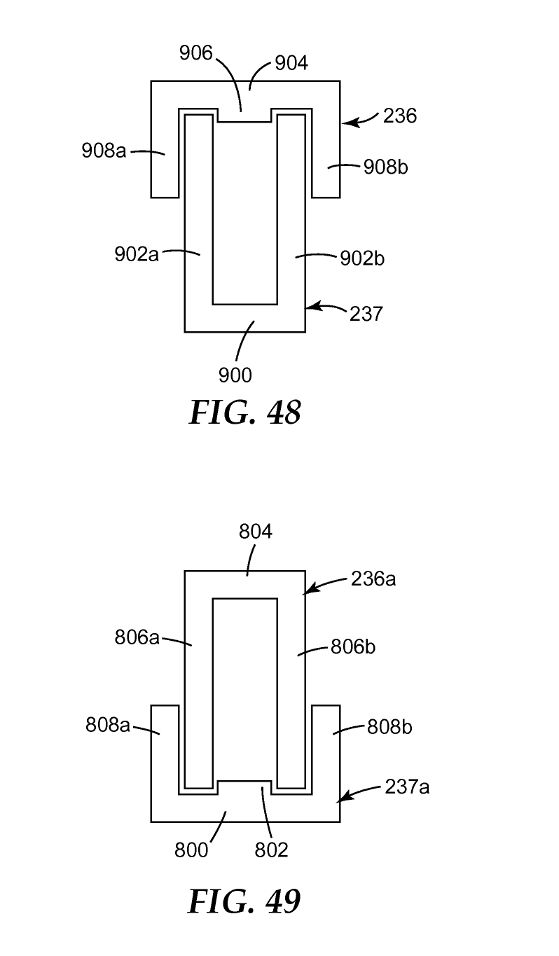

The medicament chamber piercing and air flow assembly 234 in the illustrated embodiment includes two air inlet channels 230a and 230b (see, e.g., FIGS. 34B, 35B and 36B). Each air inlet channel is defined in part by portions of the upper airways component 236 and the lower airways component 237. For instance, in the illustrated embodiment, each air inlet channel has a generally rectangular cross-sectional area, in lateral section relative to the direction of inlet air flow through that inlet channel, with a ceiling, floor and side walls. This arrangement is illustrated schematically in FIG. 48 for an illustrative air inlet channel wherein the lower airways component 237 has a floor 900 and side walls 902a and 902b (and also, to some extent, in FIGS. 34A, 35A, 36A and 37-38C). The upper airways component 236 has a ceiling 904. The ceiling 904 has a rib 906 extending downwardly from the ceiling 904 in between upper inner edges of the side walls 902a, 902b to prevent the side walls from distorting inwardly. The ceiling 904 also has downwardly depending wings 908a and 908b which extend partially along upper portions of the side walls 902a and 902b, respectively, to provide some overlap with the side walls 902a and 902b, in order to avoid significant air leaks into the inlet air channel between the lower airways component 237 and upper airways component 236. An alternative embodiment for an air inlet channel construction, with the relationships of portions of the upper and lower airway components reversed, is illustrated schematically in FIG. 49. Here, a lower airways component 237a has a floor 800. Extending upwardly from the floor is a rib 802. An upper airways component 236a has a ceiling 804 and side walls 806a and 806b. The floor 800 of the lower airways component 237a includes upwardly extending wings 808a and 808b, which extend partially along lower portions of the side walls 806a and 806b, respectively, in order to provide some overlap with the side walls 806a and 806b to avoid significant air leaks therebetween. The rib 802 is disposed between lower inner edges of the side walls 806a, 806b to prevent the side walls from distorting inwardly.

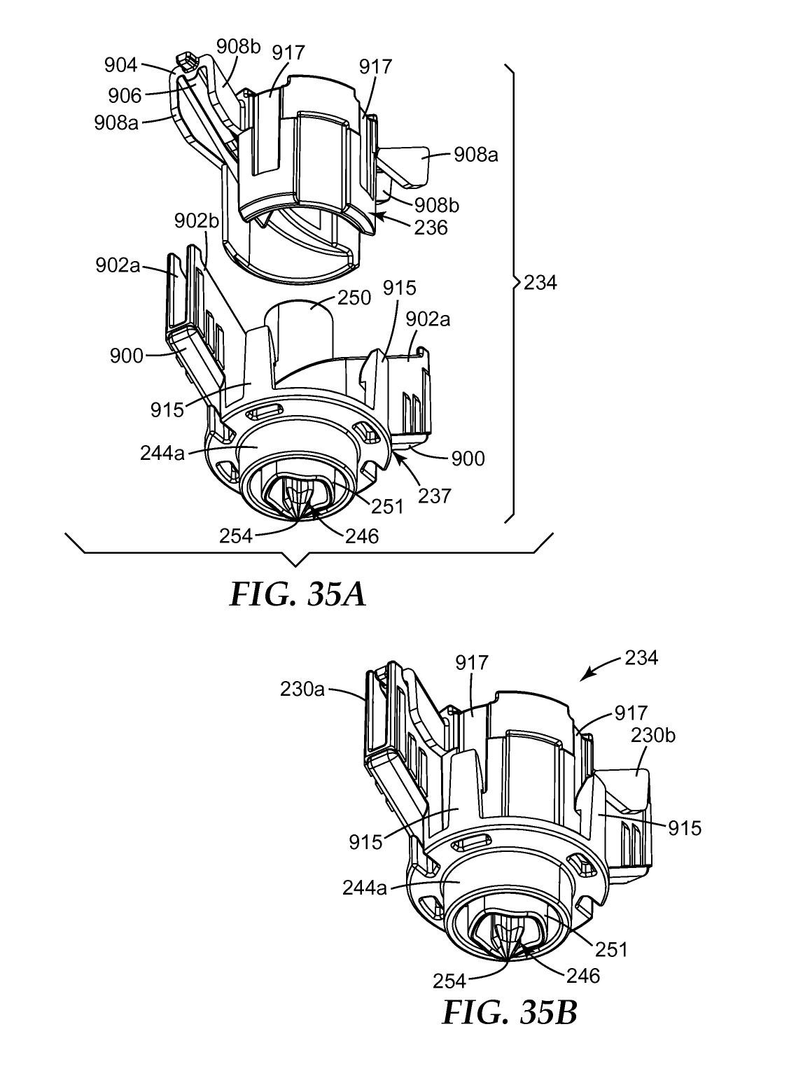

In embodiments, the lower airways component 237 is a molded single integral plastic component having a cover piecing element thereon, as indicated by cover piecing element 246 seen in FIGS. 35A, 35B, 36A-36C, 37 and 38A-38C. Likewise, the upper airways component may be a molded single integral plastic component. When combined to form the medicament chamber piercing and air flow assembly 234 the upper and lower airways components 236 and 237 are coupled together via suitable means, such as a plurality of upright resilient prongs 915 on the lower airways component that are received within corresponding recesses 917 on the upper airways component and have engaging opposed clip surfaces thereon.

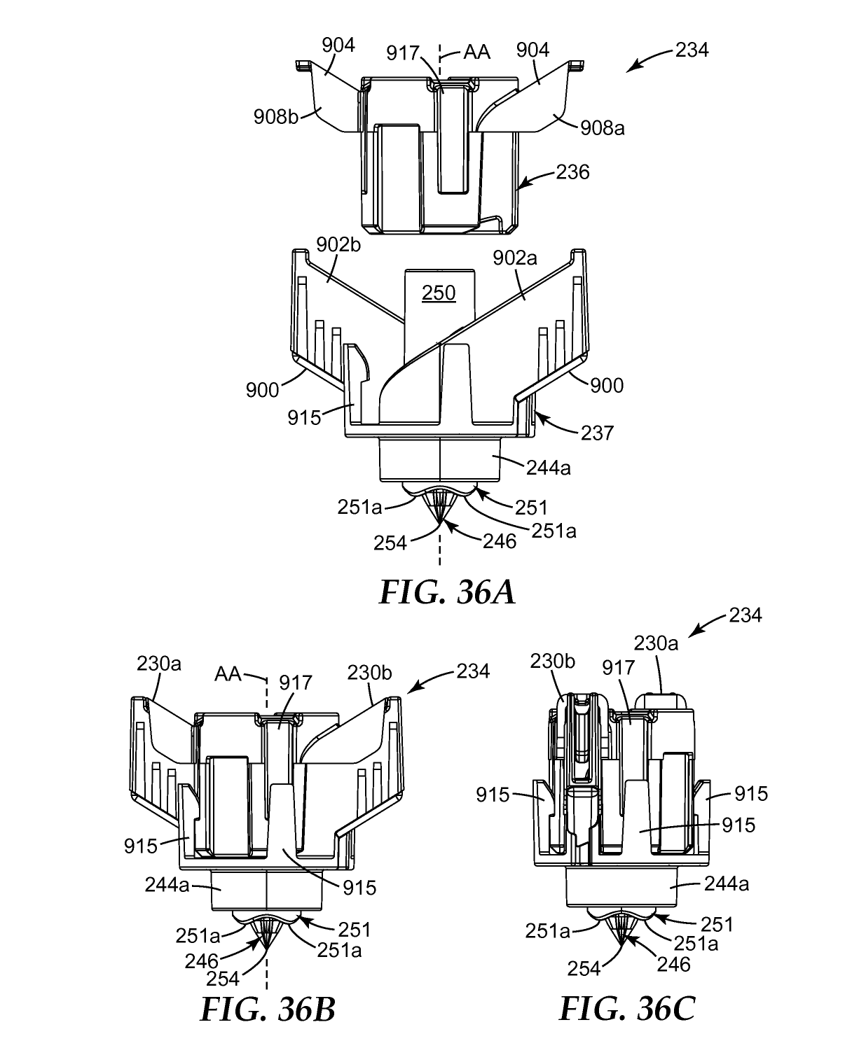

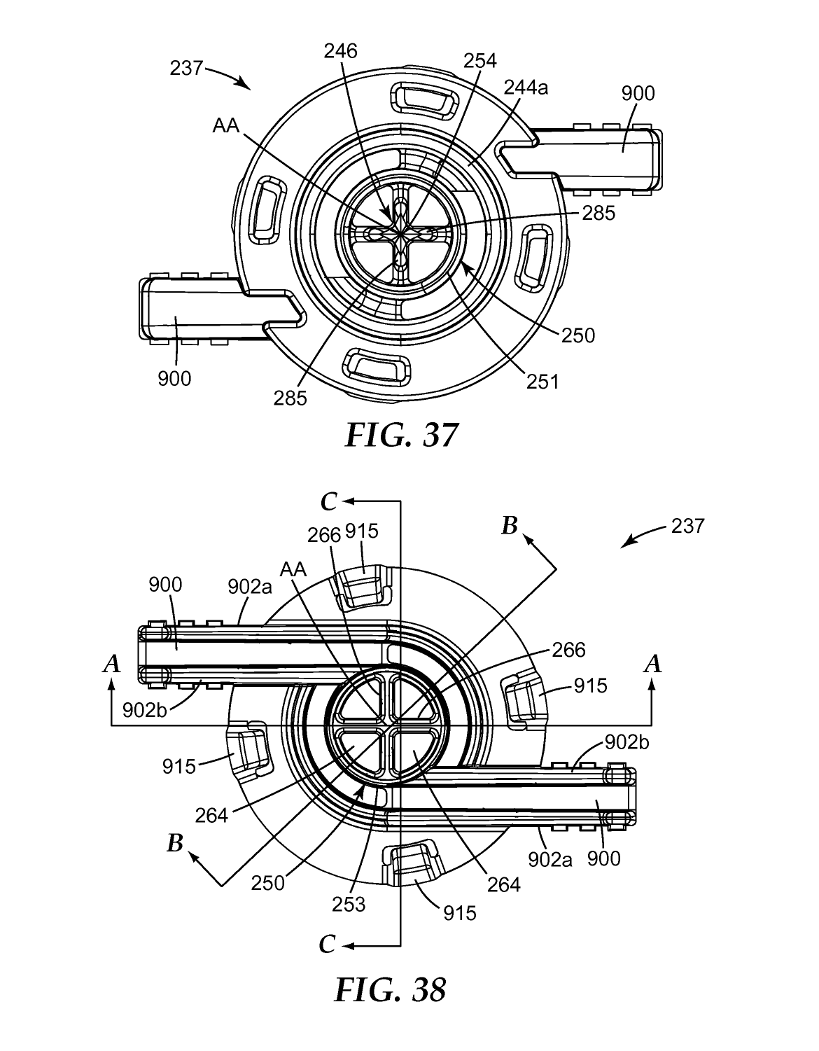

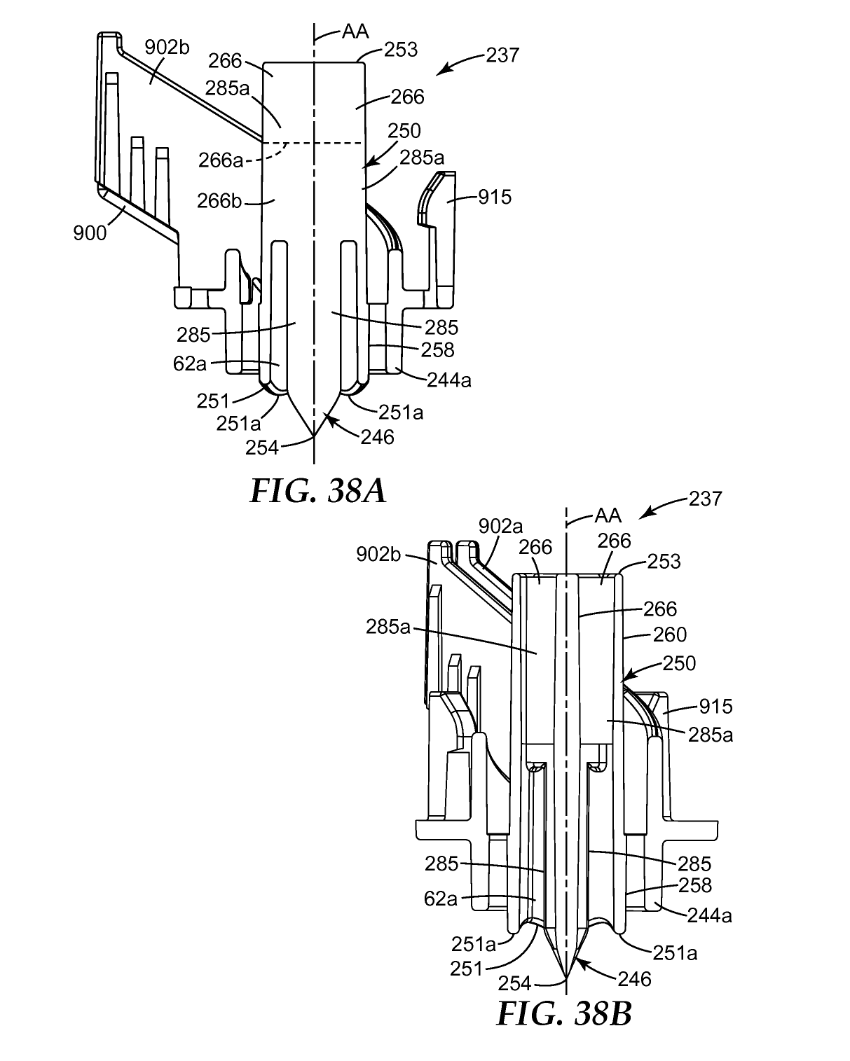

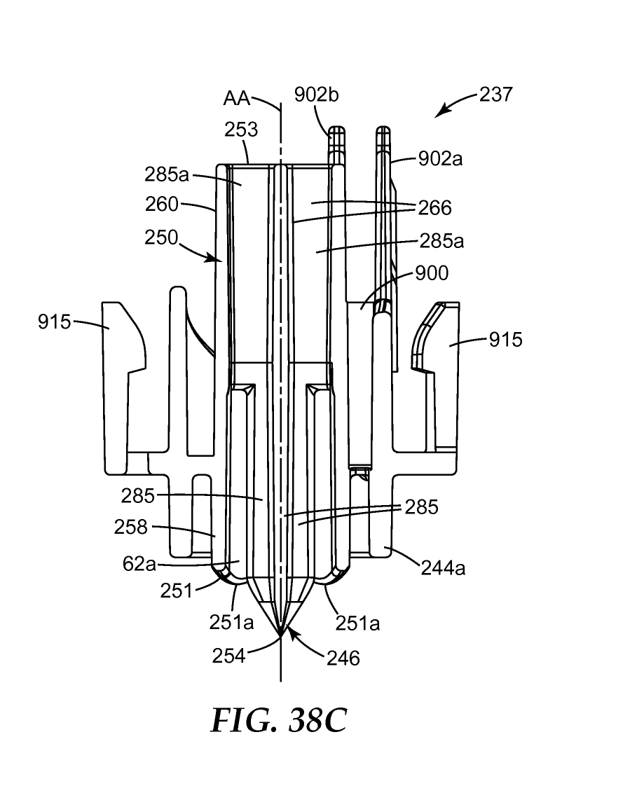

In the illustrated embodiment, an outlet tube 250 is also disposed on the lower airways component 237. The cover piecing element 246 extends outwardly from a first end 252 of the outlet tube 250. The cover piecing element 246 extends along the axis AA of the medicament chamber piercing and air flow assembly 234 such as illustrated, for example, in FIGS. 36A and 38A. The cover piecing element 246 has a central piercing point or tip 254 and a plurality of cover separation wings 285 (see FIGS. 38A-38C). In embodiments, four cover separation wings 285 are provided and aligned in a cross-like configuration. The tip 254 of the cover piecing element 246 extends along the axis AA, and each cover separation wing extends radially outwardly from the cover piecing element 246, as illustrated in a bottom view of the lower airways component 237 (FIG. 37), and in FIGS. 35A and 35B. The outlet tube 250 has a first lower section 258 and a second upper section 260. An upper portion 285a of each cover separation wing 285 extends axially away from the chamber 210 and into the second section 260 of the outlet tube 250. The upper portion 285a of each cover separation wing 285 in the second section of the outlet tube 250 extends further radially outwardly to engage an inner surface of the outlet tube 250, as illustrated in FIGS. 38A and 38C. The upper portion 285a of the cover separation wings 285 that extend further radially outwardly define walls 266 separating the second section 260 of the outlet tube 250 into a plurality of ducts 264. In embodiments, the upper portions 285a of the cover separation wings 285 do not extend along an entire length of the second section 260 of the outlet tube 250. This configuration is illustrated, for example, by the dashed line edges 266a in FIG. 38A. The dashed line edges 266a would represent top edges of the upper portions 285a (and their resultant walls 266b) thus illustrating that the divider walls 266 extend only along a portion of the second section 266 of the outlet tube 250. In a configuration where the cover separation wings 285 and their respective upper portions 285a are linear and extend continuously across at least portions of both the first and second sections 258 and 260 of the outlet tube 250, the upper portions 285a likewise are cross-shaped in the second section 260 of the outlet tube 250, like the cover separation wings 285 in the first section 258 of the outlet tube 250. As best seen in FIGS. 37 and 38, the cross-shape is in a plane normal to the axis AA. In embodiments, each cover separation wing 285 is in the form of a straight fin or vane.

Like the medicament container piercing and air flow assembly 134 of the first disclosed embodiment, the assembly 234 and its respective lower airways component 237 illustrated in FIGS. 34A-38C is configured to facilitate air flow into, within and out of a reverse cyclone chamber (i.e., chamber 210; FIGS. 32-33). The cover piecing element 246, and its associated tip 254 and wings 285 are shaped, as discussed with respect to the like assembly 134 discussed above, to engage, pierce, and separate the cover 284 over the chamber 210, thereby allowing access to the dry powder medicament particles therein. In addition, portions of the medicament chamber piercing and air flow assembly 234 extend into and reside within the chamber 210 (including the cover piecing element 246, a lower portion of one or more air inlets such as defined in part by an outer circumferential surface of the first section 258 of the outlet tube 250 and an inner circumferential surface of outer cylindrical wall 244a, and an air outlet defined in part by a duct 62a (FIGS. 38A-38C) extending through the first section 258 of the outlet tube 250).

In order to further facilitate management of flap covers of a breached and separated chamber cover 284, the first section 258 of the outlet tube 250 has a wave shape along its first edge 251, as illustrated in FIGS. 35A-36C and 38A-38C. The wave shape is formed by a plurality of cover management projections 251 a extending from the first end 251 of the outlet tube 250. Each cover management projection 251a extends parallel to the axis AA and, in embodiments, is aligned relative to adjacent cover separation wings 285 on the cover piecing element 246. For instance, each cover management projection 251a may be disposed at a relational angle of between 30 and 60 degrees around the axis AA relative to each adjacent cover separation wing 285. In embodiments, the relational angle is about 45 degrees. The wave shape of the first end 251 of the outlet tube 250, and its relation to the cover piecing element 246, is illustrated in FIGS. 35A-36C and 38A-38C.

As may be readily appreciated, the medicament chamber piercing and air flow assembly 234 is similar in both form and function to the medicament container piercing and air flow assembly 134. The air flow in, through and out of the assemblies 234 and 134, and their respective chambers 210 and 110, are such as illustrated in FIGS. 1-3B.

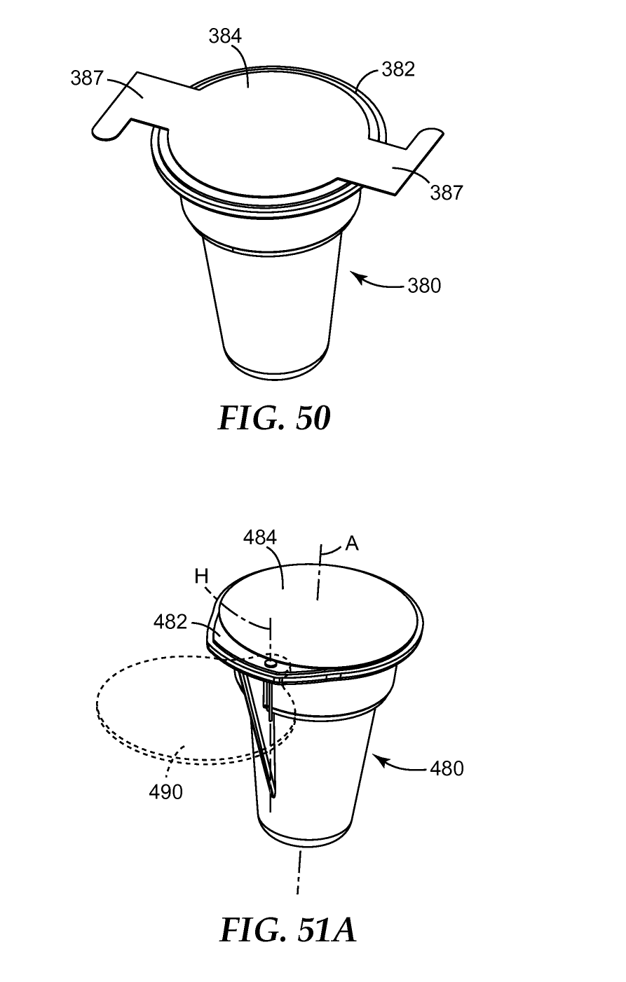

As noted above, alternative configurations for a dry powder medicament chamber, including its container and its cover, are contemplated. For instance, FIG. 50 illustrates a container 380 having a cover 384 hermetically sealed thereto. The cover 384 has one or more tabs 387 projecting radially therefrom, relative to a central axis of the container 380. Each tab 387 may serve as a cover alignment feature (to aid in assembly of the cover 384 with the container 380 (see, e.g., FIG. 31), or with respect to alignment of the container 380 bearing the cover 384 within a suitable receptacle in a dry powder inhaler assembly). Alternatively, the tab 387 may serve as a means for a user to grasp a portion of the cover 384 and peel it off from a rim 382 of the container 380, to allow access to the dry powder medicament particles therein. As depicted, the container 380 does not include an attached lid.

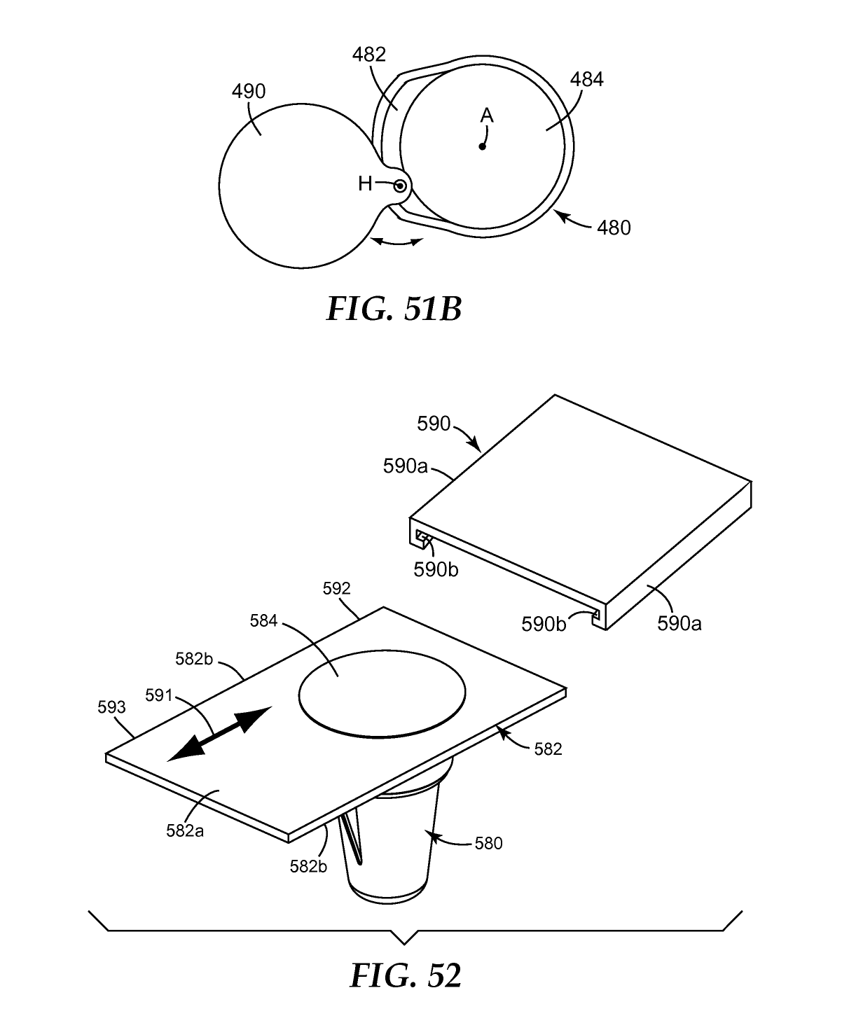

Other container alternatives include lids that differ in form and operation from the lid 90 of container 80 illustrated in FIGS. 4-7. For instance, FIGS. 51A and 51B illustrate a container 480 with a cover 484 sealably affixed to a rim 482. A lid 490 is provided, and is pivotally affixed to a portion of the rim 482 in a suitable manner such as a pivot connection or living hinge that is aligned on an axis H parallel to the axis A of the reverse cyclone chamber within the container 480. An underside of the lid 490 is planar, so that it may be pivoted about the axis H from an open position (depicted in phantom in FIG. 51A, and also depicted in FIG. 51B) to a closed position where the lid 490 is aligned over the cover 484 (which may or may not yet have been breached). Suitable means such as a detent coupling or adhesive may be provided to permit retention of the lid 490 over the chamber of the container 480 and its associated rim 482.

Another alternative container 580 is illustrated in FIG. 52. The container 580 has its rim 582 enlarged and formed as a planar flange 582a having parallel linear edges 582b along opposed sides thereof. A cover 584 is hermetically sealed to the rim 582, such as previously described. A lid 590 has opposed linear side edges 590a, each having a lower depending channel 590b formed thereon. The channels 590b are shaped to conform to and slidably mate with the side edges 582b of flange 582. The lid 590 is thus slidable along the longitudinal length of the flange 582, in direction of lid movement arrow 591. When the lid 590 is moved to a position at end 592 of the flange 582, it extends over the cover 584, and when it has been slid to an opposite end 593 of the flange 582a, the cover 584 is exposed. The movement of the cover 590 relative to the flange 582 may be done manually by a user to expose the cover 584 for use of the container 580 and, if desired, to cover the chamber of the container 580 after the cover 584 has been breached or to protect the cover 584 prior to its breach. Alternatively, movement of the cover 590 relative to the cover 584 may take place in response to a changing position of one or more other components of an inhaler in which the container 580 is disposed for use. In this particular configuration, the lid 590 thus moves substantially in a plane parallel to the plane of the cover 584 in order to selectively cover and uncover the cover 584.

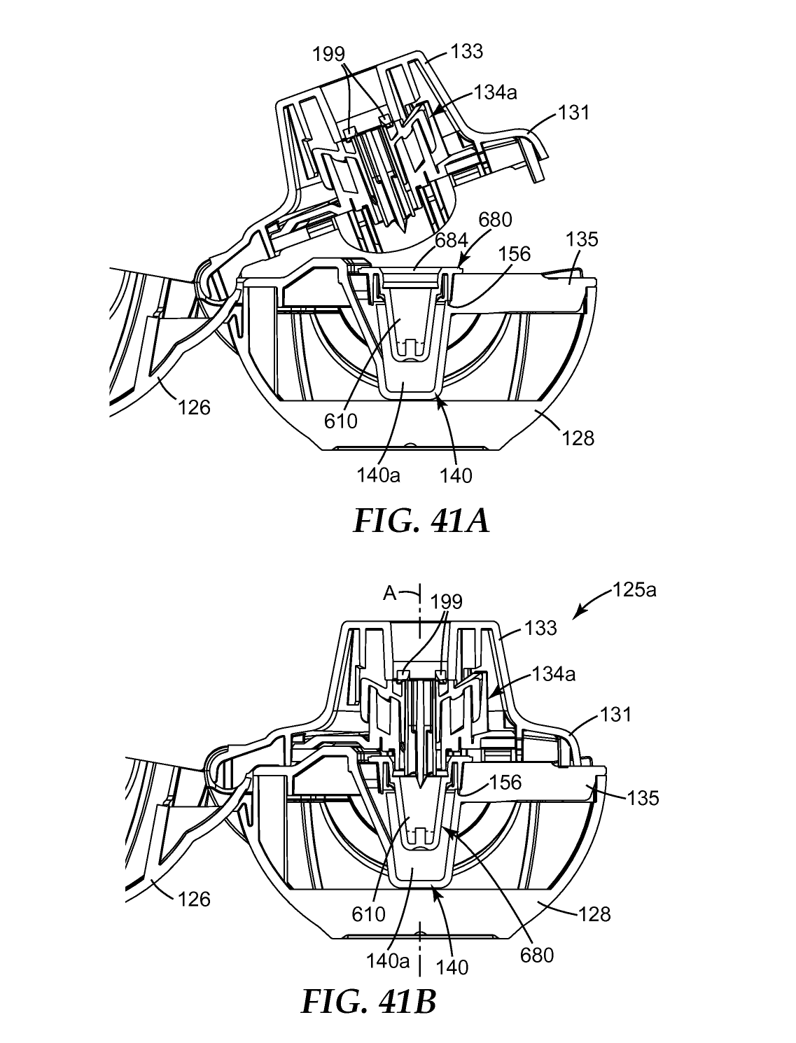

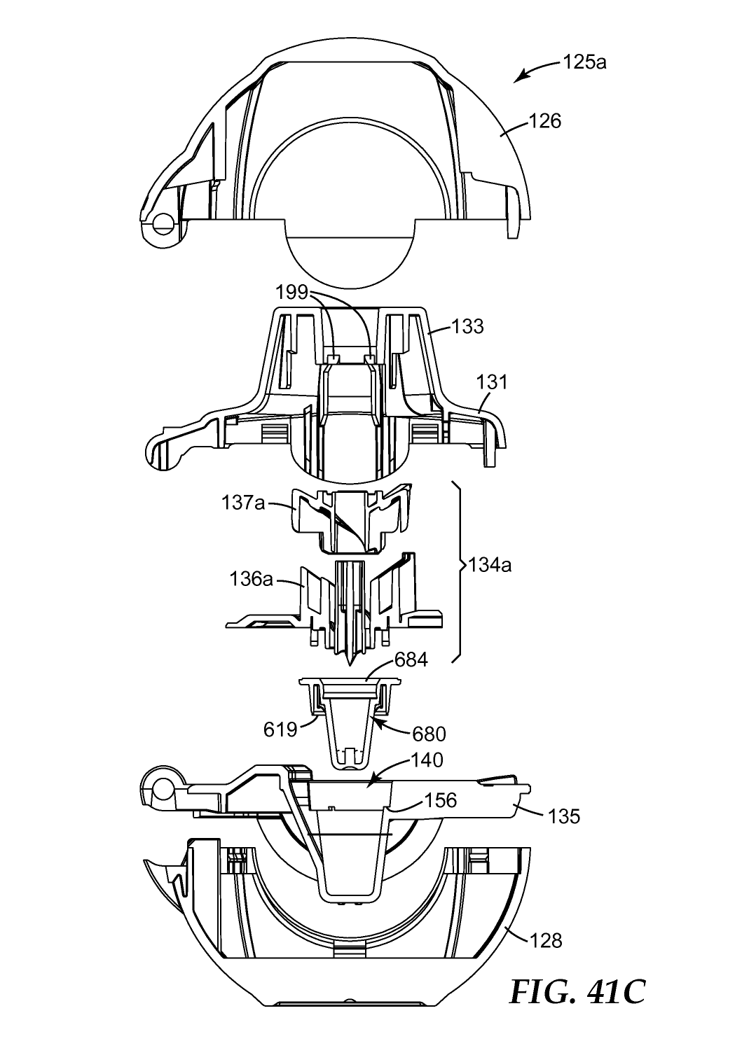

FIGS. 39-41C illustrate another alternative container 680. The container 680 has a chamber 610 therein for defining reverse cyclonic flow and other internal features for facilitating such flow, and for receiving therein and engagement thereof of a medicament chamber piercing and air flow assembly 134a (much like the assembly 134 illustrated in FIGS.41A-41C). The container 680 has an upper third portion 618 shaped otherwise the same as the third portion 118 of the container 80. However, the portion of the container 680 defining its chamber 610 is smaller in volumetric dimensions than the chamber 110 of the container 80, such as, for example in one or both of its axial and radial dimensions. The third portion 618 includes an annular shoulder 619 at a bottom edge thereof, but the shoulder 619 is supported and spaced from the chamber forming portion of the container 680 by a plurality of axially extending projections 621, as illustrated in FIG. 40. Such projections provide support for the third portion 618 and its annular shoulder 619 when the container 680 is disposed within the receptacle 140 of a dry powder inhaler assembly 125a that is operationally the same as the assembly 125 previously discussed (because the inner diameter of the reverse cyclonic flow chamber 610 in the container 680 is smaller than the chamber 110 of the container 80, the medicament container piercing and air flow assembly 134a is modified (relative to the assembly 134) to alter the flow of the inlet air (i.e., introducing each downwardly spiraling inflowing air stream in a smaller diameter)). The inner side of annular shoulder 619 of the container 680 is thus formed to selectively mate with a lower end of at least one air inlet channel extending into the chamber 610 and engages the annular shoulder 156 of the receptacle 140 (or biasing features thereon) to properly align and center the container 680 about the axis A for use (see FIG. 41B). As illustrated in FIGS. 41A and 41B, portions 140a of the receptacle 140 are not filled by the chamber 610 of the container 680, yet the container 680 is still operable in conjunction with the medicament container piercing and air flow assembly 134a of the dry powder inhaler assembly 125a to function to stir and separate dry powder medicament particles for effective delivery to a patient during use. Optionally, this smaller container 680 could also have a lid, analogous to lid 90 of container 80.

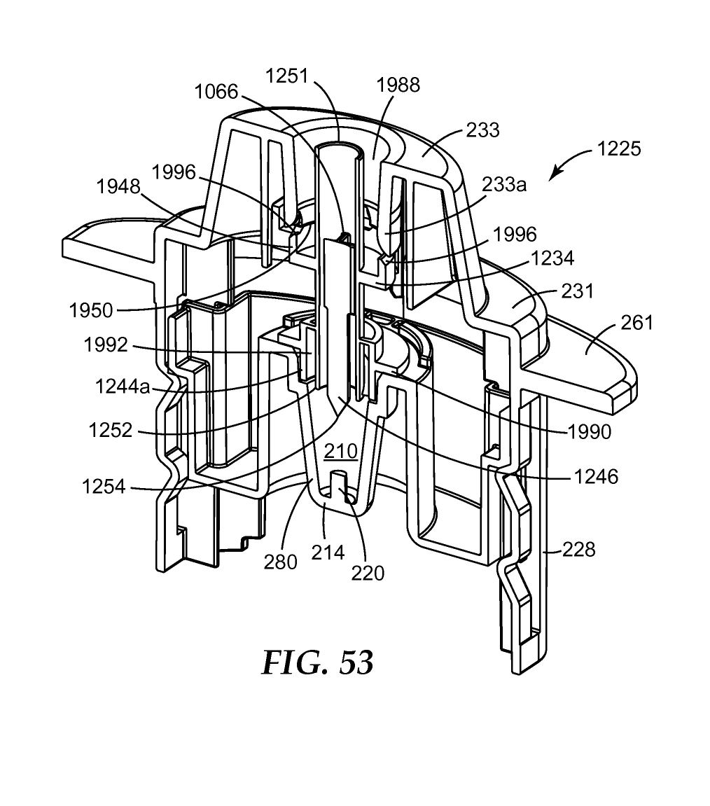

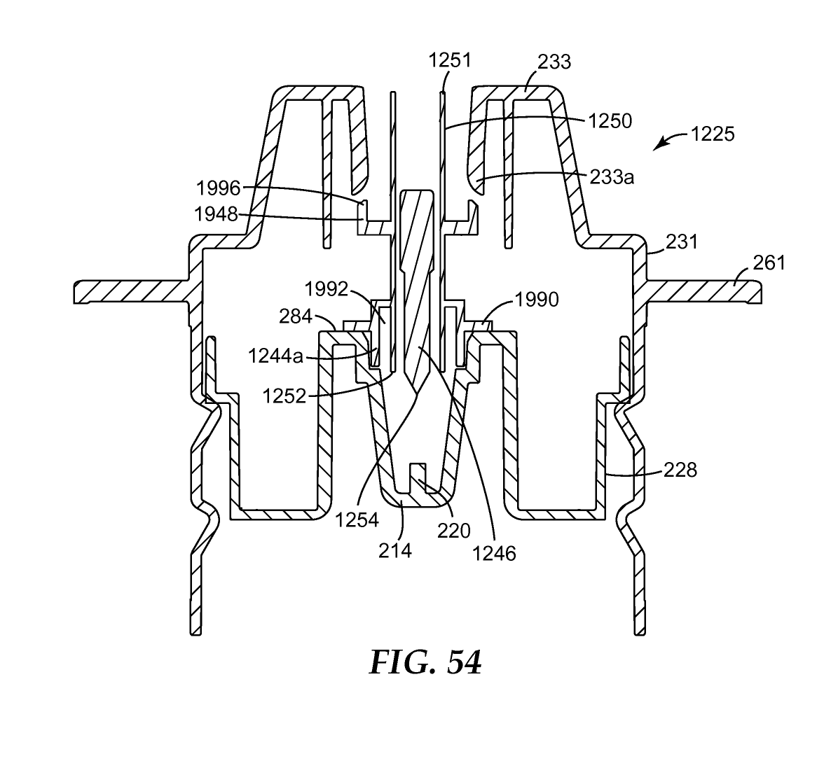

FIGS. 53-56 illustrate a further embodiment of a dry powder inhaler. Referring to FIGS. 53-54, in this embodiment, a dry powder inhaler 1225 includes a lower member 228, an upper member 231, and an airways component 1234.

In this embodiment, lower member 228 depicted in FIGS. 30-33 can be used. Lower member 228 includes a container 280 having a top 284 and a chamber 210 defined therein having a generally flat bottom wall 214 and a flow disrupter 220 projecting therefrom. As described above, chamber 210 (along with the chambers of the various embodiments depicted and described herein) may, optionally, not include a flow disruptor on bottom wall 214. Also, in this embodiment, upper member 231 depicted in FIGS. 30-33 can be used. Upper member 231 includes a mouthpiece 233 having an inner wall 233a and a radially-projecting flange 261 to further enable handling dry powder inhaler assembly 1225 during use.

Airways component 1234 is a single-component alternative to a two-component airways component in various other embodiments described herein. For example, airways component 1234 can perform the functionality of combined components 236 and 237, as depicted in FIG. 34A. In embodiments, single airways component 1234 can be a unitary, plastic component and formed using a manufacturing process, such as molding.

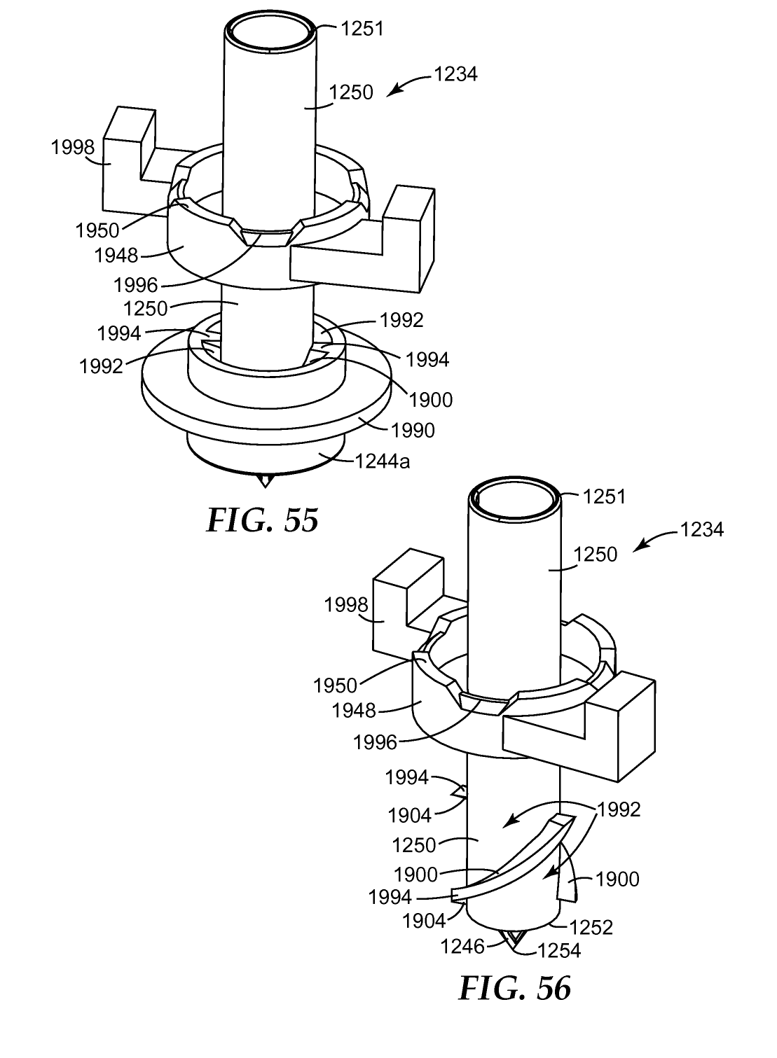

Referring to FIGS. 54-55, airways component 1234 includes a central delivery tube 1250 having an upper end 1251 and an inlet end 1252. At first, inlet, end 1252 of the central delivery tube 1250 is a cover piercing element 1246 bearing an associated tip 1254. Cover piercing element 1246 is connected to the inside of central delivery tube 1250 by four divider walls 1066 (FIG. 53). Airways component 1234 further includes an outer shroud 1948 presented therewith having an upper surface 1950 and one or more recesses 1996 (four depicted) in upper surface 1950. Also extending from outer shroud 1948 can be two fittings 1998.

Airways component 1234 also includes an outer cylindrical wall 1244a surrounding inlet end 1252 of the central delivery tube 1250. Projecting radially outwards from outer cylindrical wall 1244a is a flange 1990, which, when dry powder inhaler assembly 1225 is assembled (FIGS. 53-54), abuts top 284 of container 280 when lower member 228 is pushed upwardly relative to the upper member 231 for use. Referring to FIGS. 53-54, outer cylindrical wall 1244a is joined to the outside of the central delivery tube 1250 by one or more helical ribs 1994 (two depicted).

Referring to FIGS. 53-54, central delivery tube 1250 forms an outlet from the chamber 210 through the mouthpiece 233 of dry powder inhaler 1225. Referring to FIG. 53, when dry powder inhaler 1225 is assembled, upper surface 1950 can abut against an inner wall 233a of the mouthpiece 233. Recesses 1996 in upper surface 1950 can create air bypass inlets, which enable clean air to enter the mouthpiece 233 via an annular bypass channel 1988 formed between the central delivery tube 1250 and the inner wall 233a of the mouthpiece (FIG. 53). This clean air bypasses the powder-containing chamber 210, which enables the overall flow resistance of the dry powder inhaler to be tuned to a lower level compared to that when no bypass is included. This, in turn, improves patient comfort.

Clean air bypasses 199, 299 are also provided in the dry powder inhaler embodiment depicted in FIGS. 8-25, for example, as labeled in FIGS. 12 and 14, and in the dry powder inhaler embodiment depicted in FIGS. 30-33, for example as depicted in FIGS. 32 and 33, and in the dry powder inhaler embodiment depicted in FIGS. 41A-41C, for example as labeled in FIGS. 41A and 41C. In each of these embodiments, but not in that of FIGS. 53-54, the clean air bypasses 199, 299 can be in the form of four holes that pass clean bypass air through from the region outside the air flow assembly 134, 234 into the stream of powder-laden air from the chamber 110, 210, 610 to the outer end of the mouthpiece 133, 233. These bypass air configurations thus can enable bringing the clean air flow and the powder laden air flow together prior to the outer, downstream end of the mouthpiece. In the embodiment of FIGS. 53-54, however, the clean bypass air travels via the air bypass inlets formed by recesses 1996 and then via the annular bypass channel 1988, thus being kept separate from the powder laden air flow all the way to the outer, downstream end of the mouthpiece.

Referring again to FIGS. 53 and 54, upper end 1251 of central delivery tube 1250 can extend to the end of the mouthpiece 233. This can enable inhibition of mixing of the aerosol of dry powder particles within the central delivery tube 1250 with the bypass air that enters the mouthpiece 233 via the four air bypasses formed by recesses 1996. Also, fittings 1998 can enable single airways component 1234 to properly locate or be positioned within the upper member 231.

As discussed above, outer cylindrical wall 1244a can be joined to the outside of the central delivery tube 1250 by helical ribs 1994, which also direct the incoming air entering the chamber 210 during inhalation. In FIG. 56, outer cylindrical wall 1244a has been cut away (i.e., removed from the view) so that ribs 1994 can be seen. Ribs 1994 divide the incoming air stream into two separate streams, one in each of the helical inlet channels 1992 formed between the floor 1900 of one rib and the ceiling 1904 of the next. Each helical inlet channel can be helical for substantially its entire length. This helical air inlet arrangement has been found to provide an advantageous pattern of air to the chamber 210 in a very compact space. This embodiment also has the distinguishing characteristic that the two separate air inlet channels remain physically separated down to a point within the lower member 228, below the level of the pierceable cover 284 and into container 280. This separation enables a beneficial stratification of the two incoming air flow streams.

In embodiments, the dry powder inhalers of the present disclosure can include helical inlet channels having a pitch that varies across its width, i.e., a pitch that depends on the radius out from the longitudinal axis of the chamber. Such pitch can be arranged to be steeper on the outside side of each helical inlet channel, so as to present an air inlet angle that changes less as a function of radius from the longitudinal axis of the chamber than would otherwise be the case.

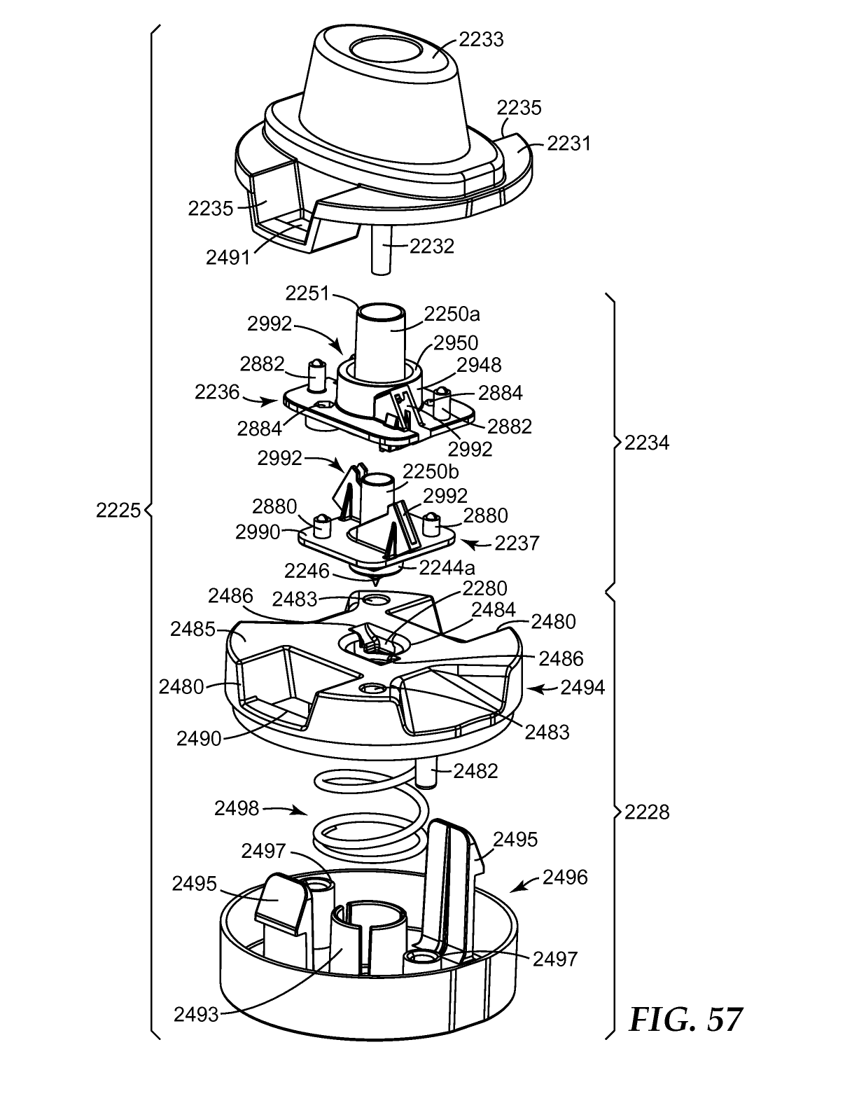

FIGS. 57-59 depict a further embodiment of a dry powder inhaler. Dry powder inhaler 2225 according to this embodiment includes a lower assembly 2228, an upper member 2231, and an airflow assembly 2234 having an upper airways component 2236 and a lower airways component 2237.

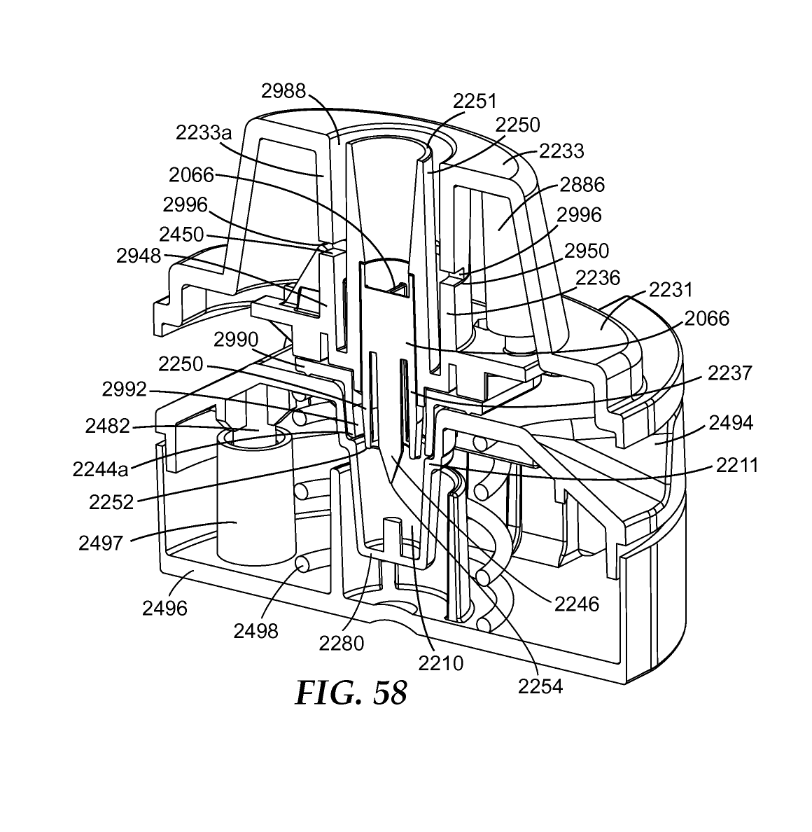

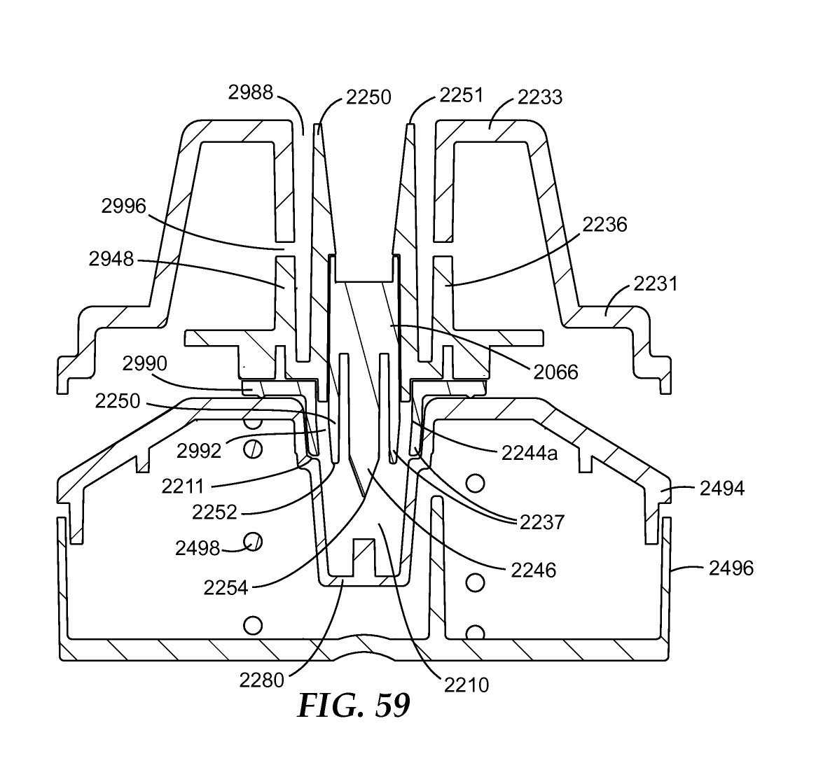

Lower assembly 2228 includes a lid member 2494, a base member 2496, and a spring member 2498 positioned therebetween, such as a metal coil compression spring. Alternatively (or additionally), spring member 2498 can also include other structures, such as metal leaf springs, metal torsion springs or integrally moulded plastic springs. Base member 2496 can include one or more clip members 2495, which can clip into and/or through apertures included in lid member 2494 (apertures 2490) and upper member 2231 (apertures 2491) thereby coupling upper member 2231 and base member 2496 of dry powder inhaler 2225 together for assembly of dry powder inhaler 2225. Base member 2496 can further include one or more positioning posts 2497 (female version depicted) for positioning lid member 2494 relative to base member 2496. A central structure 2493 can also be included in base member 2496 wherein a lower portion of a medicament container can reside when dry powder inhaler 2225 is assembled and ready for use (FIG. 58).

Lid member 2494 of lower assembly 2228 can include one or more clip apertures 2490 for operably coupling clips 2495 presented on base member 2496. Each of apertures 2490 can be located within a recess 2480 that enables nesting of an aperture member 2235 having apertures 2491 included on upper member 2231. Lid member 2494 can further include one or more positioning posts 2482 (male version depicted) for positioning lid member 2494 relative to base member 2496 and one or more positioning apertures 2483 for positioning upper member 2231 relative to lid member 2494 in conjunction with positioning posts 2232 (male version depicted) included on lid member 2494.

The spring 2498 serves to load the lid member 2494, and hence the container 2280, against the lower airways component 2237 and in turn pushes the lower airways component 2237 against the upper airways component 2236 and also tends to push the upper airways component 2236 against the upper member 2231. This spring loading serves to eliminate or reduce unwanted air leaks between the chamber 2210, the airflow assembly 2234 and the mouthpiece 2233.

Lid member 2494 of the lower assembly 2228 further includes a container 2280 comprising a chamber 2210. Referring to FIGS. 58-59, chamber 2210 has an inner annular shoulder 2211 that divides it into a lower end and an upper end, both of which have an interior form that is generally frusto-conical. Lid member 2494 further includes a recess 2484 defined on a top surface 2485 thereof with inlet recesses 2486 enabling nesting of lower portions of air inlet channels 2992 presented on lower airways component 2237.

Upper member 2231 includes a mouthpiece 2233 and clip apertures 2491 for operably coupling clips 2495 presented on base member 2496. As discussed above, aperture member 2235 can nest within recess 2480 and positioning posts 2482 (male version depicted) can be used to position lid member 2494 relative to base member 2496 when used in conjunction with positioning posts 2497.

As discussed above, airflow assembly 2234 includes upper airways component 2236 and lower airways component 2237. Airflow assembly 2234 also includes a central delivery tube 2250 that forms an outlet from chamber 2210 to the mouthpiece 2233 of the assembled dry powder inhaler 2225. Referring to FIGS. 58-59, when assembled, upper end 2251 of central delivery tube 2250 can extend to an end margin of the mouthpiece 2233, thereby inhibiting the mixing of the aerosol of dry powder particles within the central delivery tube 2250 with the bypass air that enters the mouthpiece 2233 via the bypass formed by recesses 2996.

Central delivery tube 2250 has a lower, inlet, part 2250b presented on the lower airways component 2237, that part 2250b having a cover piercing element 2246 bearing an associated tip 2254. Cover piercing element 2246 can be attached to the inside walls of the lower airways component part of the central delivery tube 2250 by four divider walls 2066. The lower, inlet, part 2250b of the central delivery tube 2250 has a lower, inlet, end 2252. The central delivery tube 2250 also has a second upper part 2250a presented on the upper airways component 2236 that, in conjunction with the inlet part 2250b, forms the outlet from chamber 2210 to the mouthpiece 2233.

Referring to FIGS. 58-59, upper airways component 2236 also includes an outer shroud 2948 formed around upper part 2250a of the central delivery tube 2250. An upper surface 2950 of outer shroud 2948 can abut against a lower end of inner wall 2233a of the mouthpiece 2233. Upper airways component 2236 also includes four recesses 2996 in the lower end of the inner wall 2233 of upper member 2231 to enable clean air to enter the mouthpiece 2233 via an annular bypass channel 2988 formed between the upper part 2250a of the central delivery tube 2250 and the inner wall 2233a of the mouthpiece 2233. This clean air bypasses the powder-containing chamber 2210 and enables the overall flow resistance of the dry powder inhaler to be tuned to a lower level than would be possible if there were to be no bypass, thus improving patient comfort.

Around the lower, inlet, part 2250b of central delivery tube 2250 is an outer frusto-conical wall 2244a. Projecting radially outwards from wall 2244a is a flange 2990, which can abut the top of container 2280 when the lower assembly 2228 is pushed upwardly relative to the upper member 2231 for use.

Upper airways component 2236 and lower airways component 2237 each have two sets of two walls, each set defining a portion of an air inlet channel 2992. The upper airways component 2236 provides the outer side walls and ceiling of each air inlet channel 2992 and the lower airways component 2237 provides the inner side walls and floor of each air inlet channel 2992. The two air inlet channels 2992 formed can be generally similar to the air inlet channels depicted in FIGS.34-35 and described in the corresponding description, i.e., each air inlet channel 2992 has an initial downwardly inclined straight section of generally rectangular cross-sectional area, leading into a downwardly inclined helical portion. In the embodiment depicted in FIGS. 57-59, the lower portion of each air inlet channel (for example, the portion indicated as 2992 in FIGS. 58-59) protrudes down into the chamber 2210 before it meets the other air inlet channel, and said lower portion is also formed between frusto-conical walls 2244a and 2250. This is in contrast to the configuration of FIGS. 35-35, where the two air inlet channels are no longer separated by walls as they enter the chamber 210 (i.e., pass below the level of the pierceable cover 284) and where they are formed between cylindrical walls. Also, as discussed above, lid member 2494 includes recess 2484 defined on a top surface 2485 thereof with inlet recesses 2486 enabling nesting of lower portions of air inlet channels 2992 presented on lower airways component 2237.

Upper airways component 2236 and lower airways component 2237 each can include positioning members (2880 on lower airways component 2237, and 2882 on upper airways component 2236) for alignment of upper airways component 2236 and lower airways component 2237 relative to other components of dry powder inhaler 2225. Upper airways component can also include a plurality of positioning apertures 2884 for alignment with positioning members 2880 on lower airways component 2237 when dry powder inhaler 2225 is assembled. Positioning members 2882 on upper airways component 2236 operably couple with female positioning members 2886 that can be included on an underside of upper member 2231 (FIG. 58).

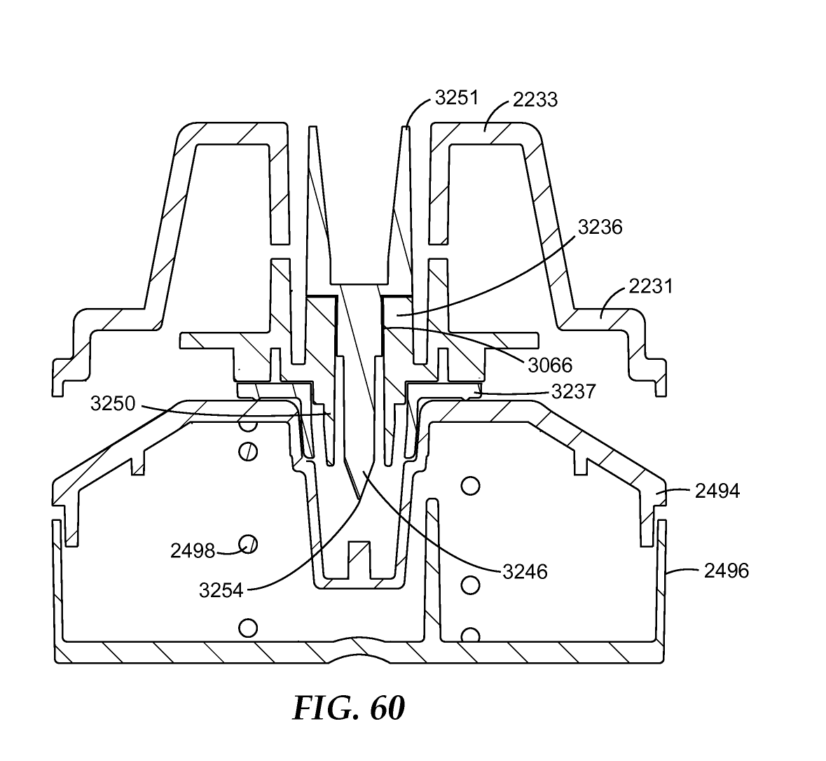

FIG. 60 illustrates a variant of the embodiment of dry powder inhaler depicted in FIGS. 57-59. The upper member 2231, base member 2496, lid member 2494, and spring member 2498 of the embodiment depicted in FIG. 60 can be the same as or substantially similar to the corresponding parts of the embodiment of FIGS. 57-59. In the embodiment depicted in FIG. 60, upper airways component 3236 of airflow assembly includes the cover piercing element 3246 with its associated tip 3254 (rather than the lower airways component 3237). In this case, cover piercing element 3246 has four divider walls 3066 that fit within the central delivery tube 3250.

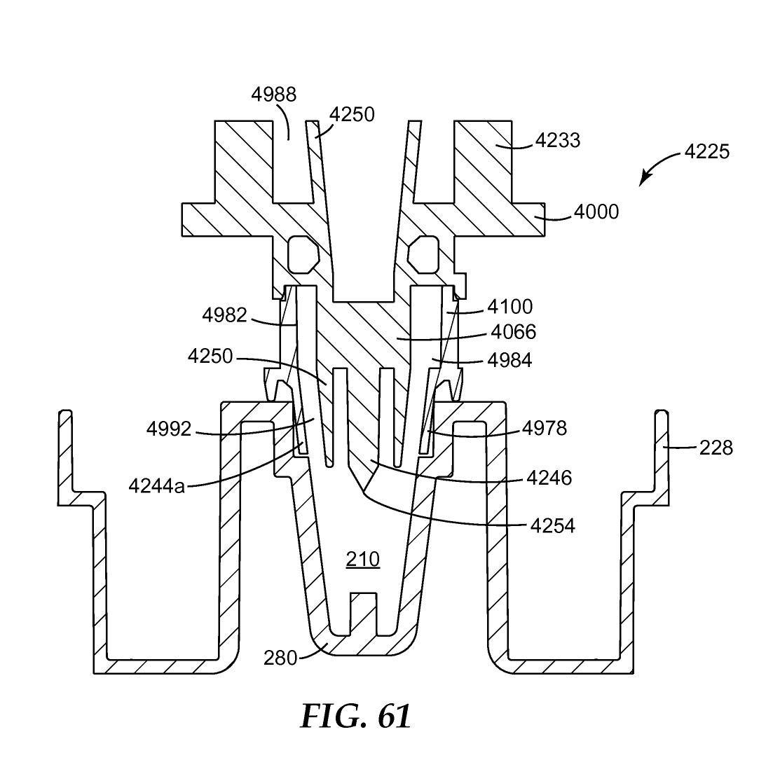

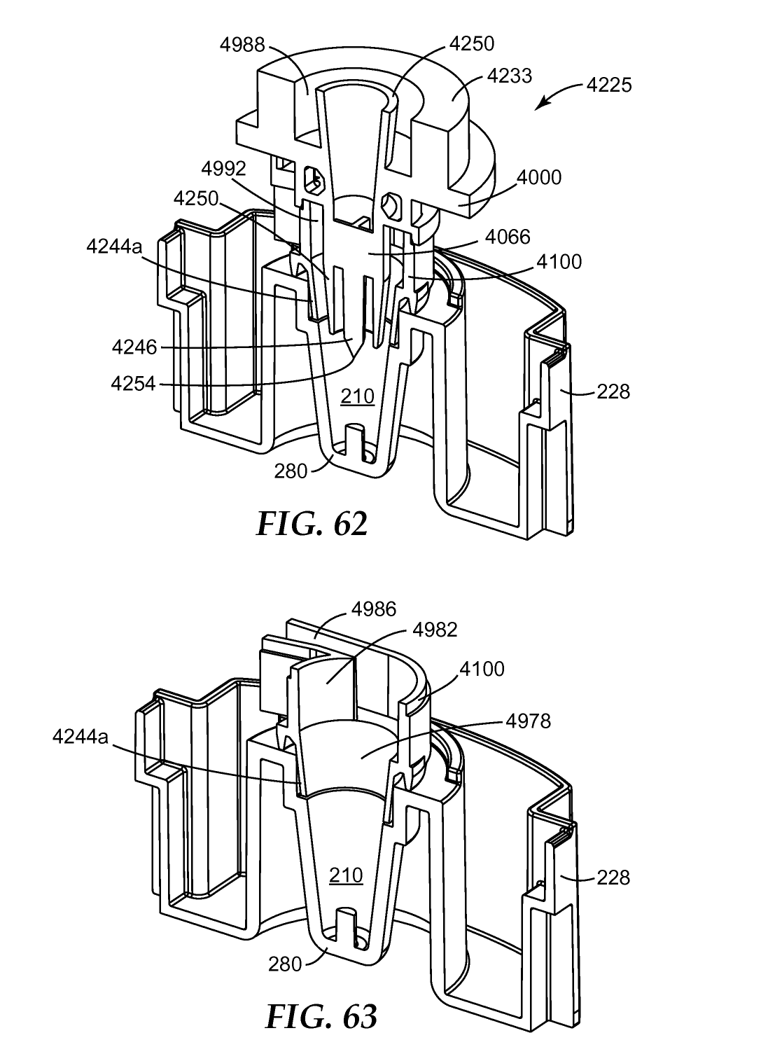

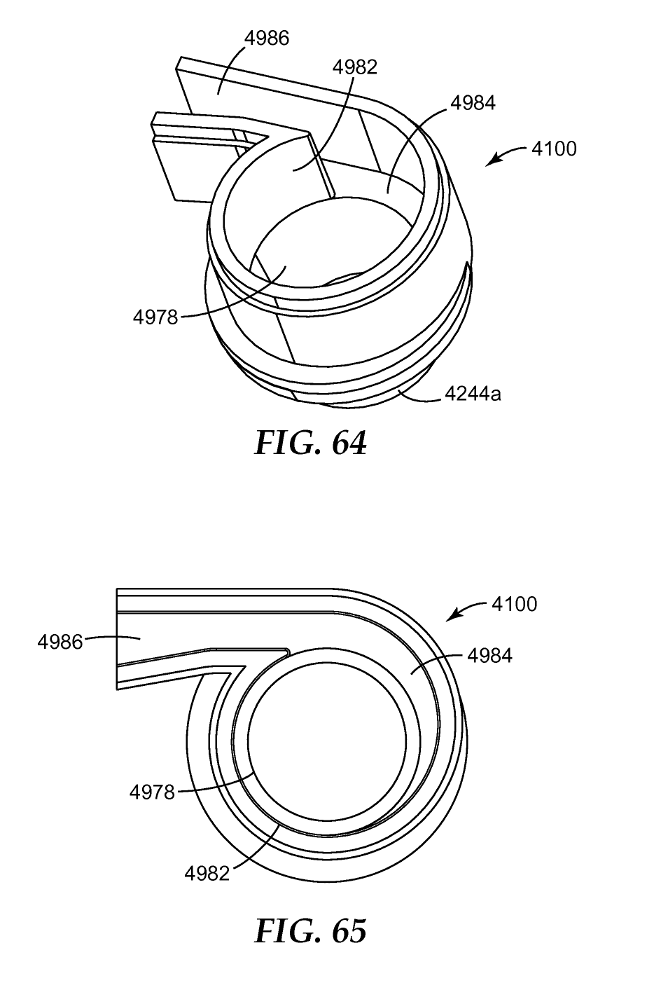

FIGS. 61-65 illustrate another embodiment of a dry powder inhaler. The dry powder inhaler 4225 depicted in FIGS. 61-62 includes a lower member 228, a lower airways component 4100, and an upper airways component 4000. FIG. 63 depicts both of the lower member 228 and the lower airways component 4100, while FIGS. 64-65 depict the lower airways component 4100 individually.

In this embodiment, lower member 228 can be the same as that denoted by the same numerals in illustrations of other embodiments of dry powder inhaler herein, such as those depicted in FIGS. 53-54.

Upper airways component 4000 in this embodiment includes a central delivery tube 4250 as an outlet from a chamber 210 to a mouthpiece 4233. Also, a cover piercing element 4246 with an associated tip 4254 is presented in central delivery tube 4250. Cover piercing element 4246 is attached to the inside walls of the central delivery tube 4250 by four divider walls 4066.

An air inlet channel 4992 is formed in the lower airways component 4100 and includes an upstream horizontal air inlet portion 4986 that narrows towards the top of the chamber. This can be seen in FIG. 65, which depicts the scroll form of the air inlet at 4984 where the air is fed into a cylindrical portion 4982 of the lower airways component 4100. Below the cylindrical portion 4982 is a conical portion 4978, the outer cylindrical wall 4244a of which mates with the upper part of the container 280 of the lower member 228, forming a continuous conical wall with the chamber 210 thereof, as can be seen in FIG. 63.

The upper airways component 4000 of this embodiment, depicted in FIGS. 61-62, engages with the lower airways component 4100 and provides the central delivery tube 4250 and also an annular opening 4988 into which multiple bypass air inlets (not depicted) open up.

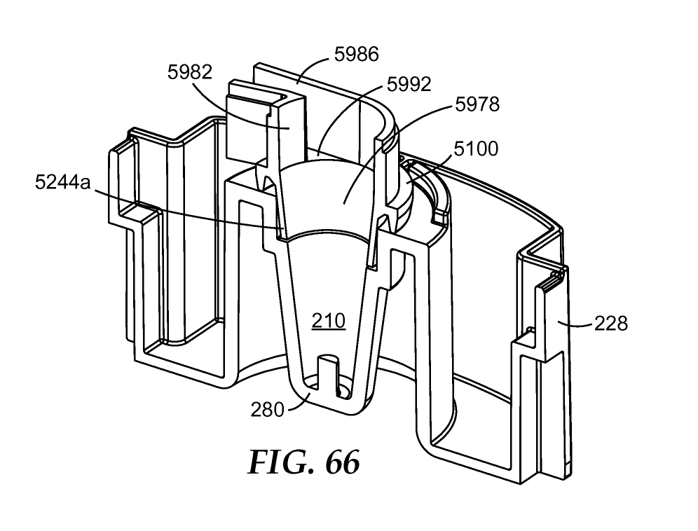

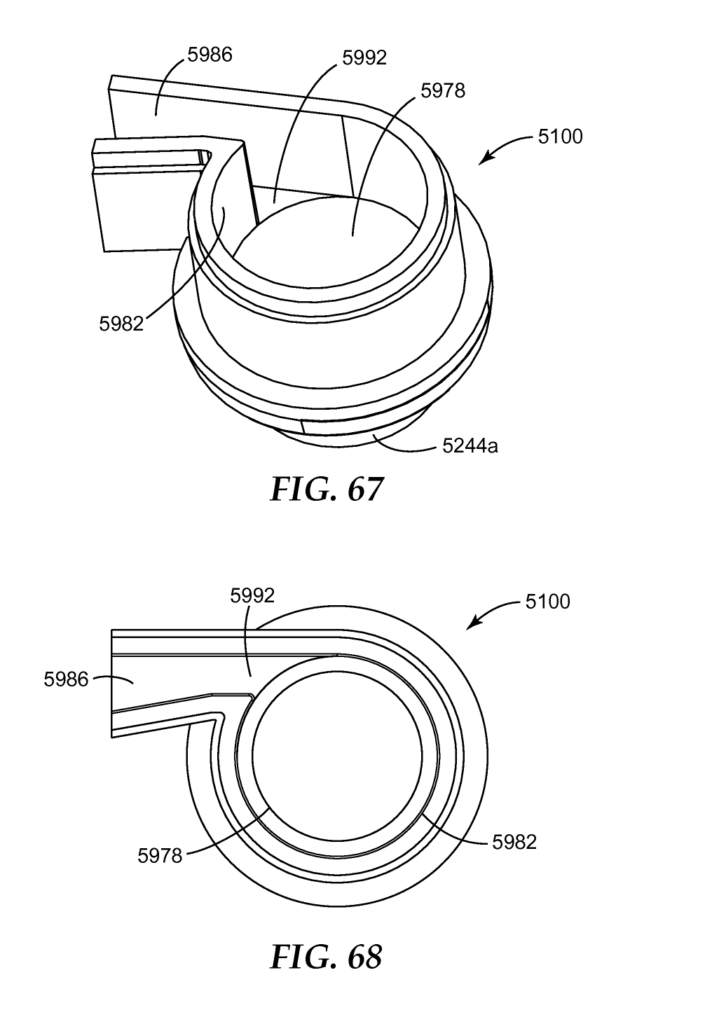

FIGS. 66-68 illustrate another embodiment of a dry powder inhaler. FIG. 66 depicts a lower member 228 and a lower airways component 5100 of the dry powder inhaler and FIGS. 67-68 depicts the lower airways component 5100, individually. The lower member 228 can be identical to that denoted by the same numerals in illustrations of other embodiments of dry powder inhaler herein, such as those depicted in FIGS. 53-54. Also, an upper airways component, such as that depicted in FIGS. 61-62, could be used with this embodiment.

The lower airways component 5100 of FIGS. 66-68 provides an air inlet channel 5992 that includes an upstream horizontal air inlet portion 5986 that narrows towards the top of the chamber. As can best be seen in FIG. 68, this embodiment of air inlet feeds air tangentially into a cylindrical portion 5982 of the lower airways component 5100. Below the cylindrical portion 5982 is a conical portion 5978, the outer cylindrical wall 5244a of which mates with the upper part of the container 280 of the lower member 228, forming a continuous conical wall with the chamber 210 thereof, as can be seen in FIG. 66.

Those skilled in the art will understand that dry powder inhaler embodiments of the present disclosure may have a different number of air inlet channels than those depicted in the Figures herein. In particular, just as an example, the dry powder inhaler embodiments illustrated in FIGS. 61-65 and FIGS. 66-68 are each depicted and described as having a single air inlet channel. In alternative embodiments, dry powder inhalers of the present disclosure have two such air inlet channels, arranged to be opposite one another around the longitudinal axis of the chamber (i.e. at 180 degrees apart). Other numbers of air inlets, e.g., three or more, are provided in alternative embodiments. These air inlets can be spaced apart in a uniform matter, i.e., three air inlets are separated by 120 degrees, four air inlets are separated by 90 degrees, and so on.

The use of reverse-flow cyclonic flow in a dry powder inhaler is described in WO2006/061637, where each cyclone has a powder storage air entry chamber, formed as a side feature in the cyclone. In use, an air inlet piercer pierces the region of foil covering the air entry chamber while an air outlet piercer (“vortex finder”) pierces the region of foil covering the center of the cyclone. The patient's inhaled breath then causes air to be drawn through the air inlet into the cyclone, setting up reverse cyclonic flow, and then drawn out through the vortex finder and patient mouthpiece. This air flow entrains the dose of powder, with the high cyclonic shearing forces causing the small drug particles in the formulation to be stripped from larger “carrier” lactose particles. The desired intention is for the drug particles to be carried into the patient's lungs and for the lactose carrier particles to be retained within the cyclone.

The present disclosure sets forth improved structure and techniques for the use of reverse-flow cyclone technology in a dry powder inhaler. For example, use of a single air inlet per cyclone can impose limitation on airflow rates possible for resistance levels comfortable to patients. Also, provision of a powder storage air entry chamber means that there may be uncertainty (and uncontrollability) in the location of the powder at the time of piercing and the time of inhalation initiation. Additionally, such an air entry chamber also requires significant space in the X-Y plane of the inhaler (with the cyclone axis defining the Z-axis).

The present disclosure sets forth an improved reverse cyclone dry powder inhaler configuration. In one particular aspect, the number of air inlets is raised from one to two, thereby allowing greater airflow rates through the cyclone for a given air inlet size and operating pressure drop. In a second aspect, the air inlets are in the form of downward-facing channels, located above the foil sealing layer until the foil is pierced, rather than in the form of channels located as side arms of the cyclone below the foil sealing layer. In a third aspect, the air inlets are in the form of channels that impart a helical element to the direction of air flow into the cyclone, thus tending to boost cyclone rotational speed and efficiency and also tending to hold the lactose powder down at the bottom of the cyclone.

Taken together, these and other aspects of the present disclosure lead to more efficient airflow patterns, reduced unwanted loss of lactose carrier powder particles, greater initial certainty of powder location and hence potentially more consistent cyclone performance, ability to re-use a single pair of air inlet channels with replaceable medicament containers in an inhaler device, and reduced X-Y ‘footprint’ of the cyclone/inlets system in an inhaler. These and other benefits and improvements will be apparent to those skilled in the art upon careful reading of this disclosure.

The illustrated embodiments of the dual air inlets of the present disclosure include a lower airways component and an upper airways component. It should be noted, however, that these illustrations are but some of the possible embodiments of the dry powder inhaler of the present disclosure and that numerous other ways of configuring the dry powder inhaler and containers therefore will be readily apparent to one skilled in the art. For example, the dual air inlets could include a single component serving the combined functions of the upper and lower airways components described herein. Such a single component could be formed by stereolithography “rapid prototyping” techniques.

The upper airways component serves the primary function of providing a roof for each of the two air inlets. It may be clipped onto the lower airways component, via clips on the latter, or it may be connected with an interference fit, or it may be loose and unattached. For example, if the lower airways component is clipped onto the component forming the mouthpiece of a dry powder inhaler according to the present disclosure, then the upper airways component may simply be trapped between the lower airways component and the mouthpiece component and might be attached to neither. It can be observed that the upper airways component has undercut recess features that receive clips from the inhaler's mouthpiece component.

The lower airways component provides the base and side walls of each air inlet passageway. In combination with the air inlet roof features provided by the upper airways component, each of the two air inlet passageways is accordingly defined. Suitably, the upper and lower airways components may be formed by injection moulding of a suitable polymer (e.g., polypropylene). Because side walls made in such a material may be relatively flexible, it may be desirable to provide features (e.g., ribs) to ensure that the side walls do not tend to distort inwards after molding or during assembly. Such ribs thus provide a means of ensuring that the two air inlet passageways remain open and unobstructed with a desired spacing therebetween.

The lower airways component also provides air outlet passageways and a piercer. The latter serves to puncture the center of the foil lid of the cyclone component that contains the dose of medicinal powder for inhalation by the patient. The powder typically sits initially around a pedestal feature that protrudes upwards from the center of the floor of the cyclone part of the cyclone component.

In addition to the pedestal feature located at the centre of the cyclone, the cyclone component also includes a foil lid hermetically sealed to its top surface. In addition, a broader rim feature (i.e., a “plant pot” rim) is provided around the upper end of the cyclone. This “plant pot” rim corresponds to the third portion 118 and its associated annular shoulder 119 of the container 80 depicted in FIGS. 4-7. While the illustrated containers herein all depict this “plant pot” rim as generally circular in radial section relative to the chamber axis, that portion of the container may assume other configurations, such as a square (or even an asymmetrical configuration). Such an alternative configuration, with appropriately shaped and arranged air inlet channels, may allow for additional space (i.e., adjacent the four corners of the square) for receiving the displaced cover flaps after the cover has been breached.

An annular skirt is located around the piercer and outlet tube on the lower airways component, and the lower end of this skirt is designed to align closely with the cyclone's internal wall when the complete inhaler is ready for use. In brief, to prepare a fully disposable inhaler for use (such as the inhaler assembly depicted in FIGS. 30-38C), the patient pushes upwards on the cyclone component, driving it upwards towards the assembly comprising the mouthpiece, and towards the upper airways and lower airways components. This relative motion causes the piercer, by virtue of its sharp tip, to penetrate (pierce) the foil lid of the cyclone component. As the relative motion continues, the pierced foil lid is pushed downwards and outwards by the outer wall of the outlet passageways (“vortex finder”). A lower end of this outer wall is scalloped, with four crowns in its wavy lower edge corresponding in rotational alignment to the four outlet passageways formed within the wall of the vortex finder. These four outlet passageways are defined between four straight walls radiating out in the shape of a cross (in cross section) from the piercer to the vortex finder. It will be appreciated that these four walls serve to support the piercer within the lower airways component. Although these four support walls extend out to the vortex finder, they do not do so along their entire lengths. Instead, the walls are restricted to a smaller “cross” shape in cross-section at their lower ends. This arrangement has the benefit, in addition to providing the aforementioned support/attachment of the piercer, of acting as a ‘swirl killer’ on the exiting air and powder mixture. The dry powder inhalers of the present disclosure create a swirling vortex of powder-bearing air, which leaves the cyclone via the outlet passageways. By providing four such outlet passageways with longitudinal walls between them, the rotation of the swirling air around the central axis of the piercer is effectively greatly reduced. This has the beneficial effect of reducing the speed (and hence the frictional energy losses) of the air/powder aerosol relative to the stationary wall of the vortex finder. As a result, the overall energy losses of the cyclone system (and hence the pressure drop that the patient must provide to get a given air flow rate through the dry powder inhaler) are reduced. However, it should be noted that excessive ‘swirl killing’ too close to the rotational air in the cyclone will reduce the strength of the vortex in the cyclone. Since this vortex is responsible for deagglomerating the small drug particles from the much larger lactose ‘carrier’ particles in the powder formulation, a weakened vortex will lead to reduced deagglomeration and hence to lessened pharmaceutical performance (in terms of drug delivery to the patient—less respirable drug particles; more non-respirable drug-on-lactose agglomerates). The smaller ‘cross’ of the four support walls at their lower (piercer tip) ends serves to reduce the degree of ‘swirl killing’ close to the vortex in the cyclone part of the cyclone component.

As mentioned previously, the four crowns on the scalloped lower edge of the outer wall of the vortex finder align with the four outlet passageways (i.e. the centers of their crowns are 45 degrees (in terms of rotational alignment) away from the four support walls). As the piercer tip punctures the foil lid of the cyclone component, these four support walls have a tendency to promote the tears that occur in the foil lid. Although these tears can be variable in number, position and form, it is not unusual for four tears to result, each associated with one of the support walls. The rotational alignment of the four crowns is then such that each crown tends to push down on one of the foil flaps that are formed between the tears. Whether or not four flaps, or a different number, are formed, the lower edge of the vortex finder's outer wall will in any case tend to push the flaps of torn foil downwards and outwards, a process encouraged by the lower edge of the annular skirt of the lower airways component. As the lower edge of this skirt approaches the lower surface of the ‘plant pot’ rim of the cyclone component, the flaps of foils will tend to be confined in the annular space between the skirt and the rim, thereby preventing them from obstructing the airways within the skirt.

A refillable dry powder inhaler (such as the inhaler assembly depicted in FIGS. 8-29) is prepared for use in similar fashion, except that the cyclone component has a different external form and is removable and replaceable after use.

With a dry powder inhaler of the present disclosure now prepared for use, the foil lid of the cyclone component has now been punctured and confined to the aforementioned annular space, and the outer skirt of the lower airways component is now generally aligned with the top of the inner wall of the cyclone part of the cyclone component. In effect, six air passageways now connect to the cyclone part of the cyclone component: four of them are outlet passageways around the central piercer, divided from one another by the ‘swirl-killing’ walls of the lower airways component. The other two, further out in diameter and divided from the four outlet passageways by the vortex finder outer wall, are the dual helical air inlets formed from the upper and lower airways components. By virtue of the geometry of these airways components, the helical inlets form air inlet passageways that deliver air tangentially and downwardly into the cyclone chamber. The helix angle of the double helical air channels so formed is picked so that each of the two incoming airstreams passes just over the top of the other as it comes in, without direct collision. This reduces unwanted turbulence in the system and hence reduces energy losses and pressure drop. In addition, the air inlets are sized so that their circumferential opening dimensions into the cyclone are as large as possible (representing 180 degrees of opening each, minus the wall thickness between them).

Those skilled in the art will recognize that the top and bottom and intermediate diameters of the chambers of the various dry powder inhaler embodiments of the present disclosure may be selected to optimize the technical performance and powder holding capacity of the chambers when used with different medicament powder formulations. Therefore, the wall angles of the chambers may also be changed, and in particular may be less steep than those shown in the figures.

Although the dry powder inhaler assembly and container disclosed herein has been described with respect to several embodiments, workers skilled in the art will recognize that changes may be made in form and detail without departing from the spirit and scope of the dry powder inhaler assembly and container disclosure.

1. A dry powder inhaler comprising:
a first member comprising a first mating surface, a first air inlet and an air outlet; and
a second member comprising a second mating surface adapted for selective engagement with the first mating surface and a chamber formed at least in part for reverse cyclone flow therein,
wherein the first and second members are moveable relative to one another between a first position wherein the mating surfaces are spaced apart and a second position wherein the mating surfaces are engaged and portions of the each of the first air inlet and air outlet are within the chamber of the second member.

2. The dry powder inhaler of claim 1 wherein the first member further comprises a cover piercing element, and wherein at least a portion of the cover piercing element is disposed within the chamber of the second member when the first and second members are in the second position.

3. The dry powder inhaler of claim 1 wherein the first member and the second member pivot relative to one another between the first and second positions.

4. The dry powder inhaler of claim 1 wherein the reverse cyclone flow occurs about an axis, and wherein the first member and the second member translate axially relative to one another between the first and second positions.

5. The dry powder inhaler of claim 2 wherein the reverse cyclone flow occurs about an axis that extends along a center line of the chamber, wherein the first member and/or the second member comprise alignment features that ensure axial movement of the cover piercing element along the axis as the members move between their first and second positions.

6. The dry powder inhaler of claim 2 wherein the chamber has a cover thereon, and wherein the first member and the second member move relative to one another such that a leading portion of the cover piercing element first contacts the cover at the center of the cover.

7. The dry powder inhaler of claim 2 wherein the reverse cyclone flow occurs about an axis that extends along a center line of the chamber, and wherein the first member and the second member move relative to one another such that a leading portion of the cover piercing element lies along the axis when the first and second members are in the second position.

8. The dry powder inhaler of claim 6 wherein the leading portion of the cover piercing element first contacts the cover when the first member is in a third position relative to the second member, the third position being between the first and second positions, wherein the first member and the second member pivot relative to one another between the first, third and second positions, respectively, and wherein the pivoting occurs around a hinge axis normal to an axis of the chamber around which the reverse cyclone flow occurs, the hinge axis being positioned at a height that is approximately midway between the height of the leading portion of the cover piercing element when the first member is in the third position and the height of the leading portion of the cover piercing element when the first member is in the second position, said heights all being measured parallel to the chamber axis.

9. The dry powder inhaler of claim 1 wherein the second member has a body portion, and wherein the chamber of the second member comprises a container separable from the body portion.

10. The dry powder inhaler of claim 9 wherein the body portion comprises a receptacle shaped to receive and retain the container.

11. The dry powder inhaler of claim 10 wherein the receptacle and the container are so shaped and arranged such that containers of more than one internal size can be suitably received and retained in the receptacle.

12. The dry powder inhaler of claim 1 wherein the container comprises a cup segment and a cover sealed thereover.

13. The dry powder inhaler of claim 1, wherein the first member comprises a second air inlet, and wherein a portion of the second air inlet is within the chamber of the second member when the first and second members are in the second position.

14. The dry powder inhaler of claim 13 wherein each air inlet is formed to define a helical flow stream for air entering the chamber from that air inlet.

15. The dry powder inhaler of claim 14 wherein the air inlets are formed to define stratified helical flow streams for air within the chamber.

16. The dry powder inhaler of claim 1 wherein the first and second members are configured to enable the first and second members to be clipped together in the second position.

17. The dry powder inhaler of claim 16 wherein the second mating surface is operably urged to be adjacent to the first mating surface by a spring member when the first and second members are in their second position.

18. The dry powder inhaler of claim 17 wherein the spring member is a metal coil spring.

19. The dry powder inhaler of claim 1, further comprising a plurality of air inlet channels, wherein each inlet channel comprises an upstream portion and a downstream portion relative to the direction of air flow through that inlet channel and into the chamber, wherein the upstream portion is in part pitched relative to the axis and has a first linear section and a second arc-shaped section having its inner surface defined by an outer circumferential surface of the outlet tube, and wherein the downstream portion is an annular coaxially extending section having its inner surface defined by the outer circumferential surface of the outlet tube.

20. The dry powder inhaler of claim 19 wherein the chamber has an inner annular shoulder and one or more portions of the air inlet channels mate with the inner annular shoulder within the chamber.
